SEQUENCE LISTING (26 sequences)

SEQ ID NO: 1 - protein, 4 aa, Homo sapiens
DEAH

SEQ ID NO: 2 - protein, 129 aa, Homo sapiens
MKTLQFFFLFCCWKAICCNSCELTNITIAIEKEECRFCISINTTWCAGYCYTRDLVYKDPARPKIQKTCTFKELVYETVRVPGCAHHADSLYTYPVATQCHCGKCDSDSTDCTVRGLGPSYCSFGEMKE

SEQ ID NO: 3 - protein, 124 aa, Homo sapiens
MPACRLGPLAAALLLSLLLFGFTLVSGTGAEKTGVCPELQADQNCTQECVSDSECADNLKCCSAGCATFCSLPNDKEGSCPQVNINFPQLGLCRDQCQVDSQCPGQMKCCRNGCGKVSCVTPNF

SEQ ID NO: 4 - protein, 22152 aa, Homo sapiens
  VARIANT 13877..13878: note = Any amino acid
  VARIANT 13880: note = Any amino acid
  VARIANT 13887: note = Any amino acid
  VARIANT 13890..13891: note = Any amino acid
  VARIANT 13893: note = Any amino acid
  VARIANT 13903: note = Any amino acid
  VARIANT 13913..13914: note = Any amino acid
  VARIANT 13916: note = Any amino acid
  VARIANT 13928..13929: note = Any amino acid
  VARIANT 13938: note = Any amino acid
  VARIANT 13940..13941: note = Any amino acid
  VARIANT 14569..14571: note = Any amino acid
  VARIANT 14575: note = Any amino acid
  VARIANT 14579: note = Any amino acid
  VARIANT 14581: note = Any amino acid
  VARIANT 14587..14591: note = Any amino acid
  VARIANT 14593..14594: note = Any amino acid
  VARIANT 14725..14727: note = Any amino acid
  VARIANT 14731: note = Any amino acid
  VARIANT 14735: note = Any amino acid
  VARIANT 14737: note = Any amino acid
  VARIANT 14743..14747: note = Any amino acid
  VARIANT 14749..14750: note = Any amino acid
  VARIANT 15661..15663: note = Any amino acid
  VARIANT 15667: note = Any amino acid
  VARIANT 15671: note = Any amino acid
  VARIANT 15673: note = Any amino acid
  VARIANT 15679..15683: note = Any amino acid
  VARIANT 15685..15686: note = Any amino acid
  VARIANT 15972..15974: note = Any amino acid
  VARIANT 15978: note = Any amino acid
  VARIANT 15982: note = Any amino acid
  VARIANT 15984: note = Any amino acid
  VARIANT 15990..15994: note = Any amino acid
  VARIANT 15996..15997: note = Any amino acid
  VARIANT 16008: note = Any amino acid
  VARIANT 16015: note = Any amino acid
  VARIANT 16017: note = Any amino acid
  VARIANT 16021: note = Any amino acid
  VARIANT 16025: note = Any amino acid
  VARIANT 16034: note = Any amino acid
  VARIANT 16037: note = Any amino acid
  VARIANT 16040: note = Any amino acid
  VARIANT 16046: note = Any amino acid
  VARIANT 16051: note = Any amino acid
  VARIANT 16053..16055: note = Any amino acid
  VARIANT 16058: note = Any amino acid
  VARIANT 16062..16063: note = Any amino acid
  VARIANT 16065: note = Any amino acid
  VARIANT 16072: note = Any amino acid
  VARIANT 16075..16076: note = Any amino acid
  VARIANT 16078: note = Any amino acid
  VARIANT 16088: note = Any amino acid
  VARIANT 16268..16269: note = Any amino acid
  VARIANT 16278: note = Any amino acid
  VARIANT 16280..16281: note = Any amino acid
  VARIANT 16373..16374: note = Any amino acid
  VARIANT 16376: note = Any amino acid
  VARIANT 16383: note = Any amino acid
  VARIANT 16386..16387: note = Any amino acid
  VARIANT 16389: note = Any amino acid
  VARIANT 16399: note = Any amino acid
  VARIANT 16409..16410: note = Any amino acid
  VARIANT 16412: note = Any amino acid
  VARIANT 16424..16425: note = Any amino acid
  VARIANT 16434: note = Any amino acid
  VARIANT 16436..16437: note = Any amino acid
  VARIANT 16439..16441: note = Any amino acid
  VARIANT 16445: note = Any amino acid
  VARIANT 16449: note = Any amino acid
  VARIANT 16451: note = Any amino acid
  VARIANT 16457..16461: note = Any amino acid
  VARIANT 16463..16464: note = Any amino acid
  VARIANT 16841..16842: note = Any amino acid
  VARIANT 16844: note = Any amino acid
  VARIANT 16851: note = Any amino acid
  VARIANT 16854..16855: note = Any amino acid
  VARIANT 16857: note = Any amino acid
  VARIANT 16867: note = Any amino acid
  VARIANT 16877..16878: note = Any amino acid
  VARIANT 16880: note = Any amino acid
  VARIANT 16892..16893: note = Any amino acid
  VARIANT 16902: note = Any amino acid
  VARIANT 16904..16905: note = Any amino acid
  VARIANT 16907..16909: note = Any amino acid
  VARIANT 16913: note = Any amino acid
  VARIANT 16917: note = Any amino acid
  VARIANT 16919: note = Any amino acid
  VARIANT 16925..16929: note = Any amino acid
  VARIANT 16931..16932: note = Any amino acid
  VARIANT 17465..17466: note = Any amino acid
  VARIANT 17468: note = Any amino acid
  VARIANT 17475: note = Any amino acid
  VARIANT 17478..17479: note = Any amino acid
  VARIANT 17481: note = Any amino acid
  VARIANT 17491: note = Any amino acid
  VARIANT 17501..17502: note = Any amino acid
  VARIANT 17504: note = Any amino acid
  VARIANT 17516..17517: note = Any amino acid
  VARIANT 17526: note = Any amino acid
  VARIANT 17528..17529: note = Any amino acid
  VARIANT 17531..17533: note = Any amino acid
  VARIANT 17537: note = Any amino acid
  VARIANT 17541: note = Any amino acid
  VARIANT 17543: note = Any amino acid
  VARIANT 17549..17553: note = Any amino acid
  VARIANT 17555..17556: note = Any amino acid
  VARIANT 17567: note = Any amino acid
  VARIANT 17574: note = Any amino acid
  VARIANT 17576: note = Any amino acid
  VARIANT 17580: note = Any amino acid
  VARIANT 17584: note = Any amino acid
  VARIANT 17593: note = Any amino acid
  VARIANT 17596: note = Any amino acid
  VARIANT 17599: note = Any amino acid
  VARIANT 17605: note = Any amino acid
  VARIANT 17610: note = Any amino acid
  VARIANT 17612..17614: note = Any amino acid
  VARIANT 17617: note = Any amino acid
  VARIANT 17621..17622: note = Any amino acid
  VARIANT 17624: note = Any amino acid
  VARIANT 17631: note = Any amino acid
  VARIANT 17777..17778: note = Any amino acid
  VARIANT 17780: note = Any amino acid
  VARIANT 17787: note = Any amino acid
  VARIANT 17790..17791: note = Any amino acid
  VARIANT 17793: note = Any amino acid
  VARIANT 17803: note = Any amino acid
  VARIANT 17813..17814: note = Any amino acid
  VARIANT 17816: note = Any amino acid
  VARIANT 17828..17829: note = Any amino acid
  VARIANT 17838: note = Any amino acid
  VARIANT 17840..17841: note = Any amino acid
  VARIANT 17843..17845: note = Any amino acid
  VARIANT 17849: note = Any amino acid
  VARIANT 17853: note = Any amino acid
  VARIANT 17855: note = Any amino acid
  VARIANT 17861..17865: note = Any amino acid
  VARIANT 17867..17868: note = Any amino acid
  VARIANT 17879: note = Any amino acid
  VARIANT 17886: note = Any amino acid
  VARIANT 17888: note = Any amino acid
  VARIANT 17892: note = Any amino acid
  VARIANT 17896: note = Any amino acid
  VARIANT 17905: note = Any amino acid
  VARIANT 17908: note = Any amino acid
  VARIANT 17911: note = Any amino acid
  VARIANT 17917: note = Any amino acid
  VARIANT 17922: note = Any amino acid
  VARIANT 17924..17926: note = Any amino acid
  VARIANT 17929: note = Any amino acid
  VARIANT 17933..17934: note = Any amino acid
  VARIANT 17936: note = Any amino acid
  VARIANT 17943: note = Any amino acid
  VARIANT 17946..17947: note = Any amino acid
  VARIANT 17949: note = Any amino acid
  VARIANT 17959: note = Any amino acid
  VARIANT 18089..18090: note = Any amino acid
  VARIANT 18092: note = Any amino acid
  VARIANT 18099: note = Any amino acid
  VARIANT 18102..18103: note = Any amino acid
  VARIANT 18105: note = Any amino acid
  VARIANT 18115: note = Any amino acid
  VARIANT 18125..18126: note = Any amino acid
  VARIANT 18128: note = Any amino acid
  VARIANT 18140..18141: note = Any amino acid
  VARIANT 18150: note = Any amino acid
  VARIANT 18152..18153: note = Any amino acid
  VARIANT 18155..18157: note = Any amino acid
  VARIANT 18161: note = Any amino acid
  VARIANT 18165: note = Any amino acid
  VARIANT 18167: note = Any amino acid
  VARIANT 18173..18177: note = Any amino acid
  VARIANT 18179..18180: note = Any amino acid
  VARIANT 18191: note = Any amino acid
  VARIANT 18198: note = Any amino acid
  VARIANT 18200: note = Any amino acid
  VARIANT 18204: note = Any amino acid
  VARIANT 18208: note = Any amino acid
  VARIANT 18217: note = Any amino acid
  VARIANT 18220: note = Any amino acid
  VARIANT 18223: note = Any amino acid
  VARIANT 18229: note = Any amino acid
  VARIANT 18234: note = Any amino acid
  VARIANT 18236..18238: note = Any amino acid
  VARIANT 18241: note = Any amino acid
  VARIANT 18245..18246: note = Any amino acid
  VARIANT 18248: note = Any amino acid
  VARIANT 18255: note = Any amino acid
  VARIANT 18258..18259: note = Any amino acid
  VARIANT 18261: note = Any amino acid
  VARIANT 18271: note = Any amino acid
  VARIANT 18401..18402: note = Any amino acid
  VARIANT 18404: note = Any amino acid
  VARIANT 18411: note = Any amino acid
  VARIANT 18414..18415: note = Any amino acid
  VARIANT 18417: note = Any amino acid
  VARIANT 18427: note = Any amino acid
  VARIANT 18437..18438: note = Any amino acid
  VARIANT 18440: note = Any amino acid
  VARIANT 18452..18453: note = Any amino acid
  VARIANT 18462: note = Any amino acid
  VARIANT 18464..18465: note = Any amino acid
  VARIANT 18467..18469: note = Any amino acid
  VARIANT 18473: note = Any amino acid
  VARIANT 18477: note = Any amino acid
  VARIANT 18479: note = Any amino acid
  VARIANT 18485..18489: note = Any amino acid
  VARIANT 18491..18492: note = Any amino acid
  VARIANT 18503: note = Any amino acid
  VARIANT 18510: note = Any amino acid
  VARIANT 18512: note = Any amino acid
  VARIANT 18516: note = Any amino acid
  VARIANT 18520: note = Any amino acid
  VARIANT 18529: note = Any amino acid
  VARIANT 18532: note = Any amino acid
  VARIANT 18535: note = Any amino acid
  VARIANT 18541: note = Any amino acid
  VARIANT 18546: note = Any amino acid
  VARIANT 18548..18550: note = Any amino acid
  VARIANT 18553: note = Any amino acid
  VARIANT 18557..18558: note = Any amino acid
  VARIANT 18560: note = Any amino acid
  VARIANT 18567: note = Any amino acid
  VARIANT 18570..18571: note = Any amino acid
  VARIANT 18573: note = Any amino acid
  VARIANT 18583: note = Any amino acid
  VARIANT 18713..18714: note = Any amino acid
  VARIANT 18716: note = Any amino acid
  VARIANT 18723: note = Any amino acid
  VARIANT 18726..18727: note = Any amino acid
  VARIANT 18729: note = Any amino acid
  VARIANT 18739: note = Any amino acid
  VARIANT 18749..18750: note = Any amino acid
  VARIANT 18752: note = Any amino acid
  VARIANT 18764..18765: note = Any amino acid
  VARIANT 18774: note = Any amino acid
  VARIANT 18776..18777: note = Any amino acid
  VARIANT 18779..18781: note = Any amino acid
  VARIANT 18785: note = Any amino acid
  VARIANT 18789: note = Any amino acid
  VARIANT 18791: note = Any amino acid
  VARIANT 18797..18801: note = Any amino acid
  VARIANT 18803..18804: note = Any amino acid
  VARIANT 18815: note = Any amino acid
  VARIANT 18822: note = Any amino acid
  VARIANT 18824: note = Any amino acid
  VARIANT 18828: note = Any amino acid
  VARIANT 18832: note = Any amino acid
  VARIANT 18841: note = Any amino acid
  VARIANT 18844: note = Any amino acid
  VARIANT 18847: note = Any amino acid
  VARIANT 18853: note = Any amino acid
  VARIANT 18858: note = Any amino acid
  VARIANT 18860..18862: note = Any amino acid
  VARIANT 18865: note = Any amino acid
  VARIANT 18869..18870: note = Any amino acid
  VARIANT 18872: note = Any amino acid
  VARIANT 18879: note = Any amino acid
  VARIANT 18882..18883: note = Any amino acid
  VARIANT 18885: note = Any amino acid
  VARIANT 18895: note = Any amino acid
  VARIANT 19091..19093: note = Any amino acid
  VARIANT 19097: note = Any amino acid
  VARIANT 19101: note = Any amino acid
  VARIANT 19103: note = Any amino acid
  VARIANT 19109..19113: note = Any amino acid
  VARIANT 19115..19116: note = Any amino acid
  VARIANT 19127: note = Any amino acid
  VARIANT 19134: note = Any amino acid
  VARIANT 19136: note = Any amino acid
  VARIANT 19140: note = Any amino acid
  VARIANT 19144: note = Any amino acid
  VARIANT 19153: note = Any amino acid
  VARIANT 19156: note = Any amino acid
  VARIANT 19159: note = Any amino acid
  VARIANT 19165: note = Any amino acid
  VARIANT 19170: note = Any amino acid
  VARIANT 19172..19174: note = Any amino acid
  VARIANT 19177: note = Any amino acid
  VARIANT 19181..19182: note = Any amino acid
  VARIANT 19184: note = Any amino acid
  VARIANT 19191: note = Any amino acid
  VARIANT 19194..19195: note = Any amino acid
  VARIANT 19197: note = Any amino acid
  VARIANT 19207: note = Any amino acid
  VARIANT 19337..19338: note = Any amino acid
  VARIANT 19340: note = Any amino acid
  VARIANT 19347: note = Any amino acid
  VARIANT 19350..19351: note = Any amino acid
  VARIANT 19353: note = Any amino acid
  VARIANT 19363: note = Any amino acid
  VARIANT 19373..19374: note = Any amino acid
  VARIANT 19376: note = Any amino acid
  VARIANT 19388..19389: note = Any amino acid
  VARIANT 19398: note = Any amino acid
  VARIANT 19400..19401: note = Any amino acid
  VARIANT 19403..19405: note = Any amino acid
  VARIANT 19409: note = Any amino acid
  VARIANT 19413: note = Any amino acid
  VARIANT 19415: note = Any amino acid
  VARIANT 19421..19425: note = Any amino acid
  VARIANT 19427..19428: note = Any amino acid
  VARIANT 19439: note = Any amino acid
  VARIANT 19446: note = Any amino acid
  VARIANT 19448: note = Any amino acid
  VARIANT 19452: note = Any amino acid
  VARIANT 19456: note = Any amino acid
  VARIANT 19465: note = Any amino acid
  VARIANT 19468: note = Any amino acid
  VARIANT 19471: note = Any amino acid
  VARIANT 19477: note = Any amino acid
  VARIANT 19482: note = Any amino acid
  VARIANT 19484..19486: note = Any amino acid
  VARIANT 19489: note = Any amino acid
  VARIANT 19493..19494: note = Any amino acid
  VARIANT 19496: note = Any amino acid
  VARIANT 19503: note = Any amino acid
  VARIANT 19506..19507: note = Any amino acid
  VARIANT 19509: note = Any amino acid
  VARIANT 19519: note = Any amino acid
  VARIANT 19649..19650: note = Any amino acid
  VARIANT 19652: note = Any amino acid
  VARIANT 19659: note = Any amino acid
  VARIANT 19662..19663: note = Any amino acid
  VARIANT 19665: note = Any amino acid
  VARIANT 19675: note = Any amino acid
  VARIANT 19685..19686: note = Any amino acid
  VARIANT 19688: note = Any amino acid
  VARIANT 19700..19701: note = Any amino acid
  VARIANT 19710: note = Any amino acid
  VARIANT 19712..19713: note = Any amino acid
  VARIANT 19715..19717: note = Any amino acid
  VARIANT 19721: note = Any amino acid
  VARIANT 19725: note = Any amino acid
  VARIANT 19727: note = Any amino acid
  VARIANT 19733..19737: note = Any amino acid
  VARIANT 19739..19740: note = Any amino acid
  VARIANT 19751: note = Any amino acid
  VARIANT 19758: note = Any amino acid
  VARIANT 19760: note = Any amino acid
  VARIANT 19764: note = Any amino acid
  VARIANT 19768: note = Any amino acid
  VARIANT 19777: note = Any amino acid
  VARIANT 19780: note = Any amino acid
  VARIANT 19783: note = Any amino acid
  VARIANT 19789: note = Any amino acid
  VARIANT 19794: note = Any amino acid
  VARIANT 19796..19798: note = Any amino acid
  VARIANT 19801: note = Any amino acid
  VARIANT 19805..19806: note = Any amino acid
  VARIANT 19808: note = Any amino acid
  VARIANT 19815: note = Any amino acid
  VARIANT 19818..19819: note = Any amino acid
  VARIANT 19821: note = Any amino acid
  VARIANT 19831: note = Any amino acid
  VARIANT 19960..19961: note = Any amino acid
  VARIANT 19963: note = Any amino acid
  VARIANT 19970: note = Any amino acid
  VARIANT 19973..19974: note = Any amino acid
  VARIANT 19976: note = Any amino acid
  VARIANT 19986: note = Any amino acid
  VARIANT 19996..19997: note = Any amino acid
  VARIANT 19999: note = Any amino acid
  VARIANT 20011..20012: note = Any amino acid
  VARIANT 20021: note = Any amino acid
  VARIANT 20023..20024: note = Any amino acid
  VARIANT 20026..20028: note = Any amino acid
  VARIANT 20032: note = Any amino acid
  VARIANT 20036: note = Any amino acid
  VARIANT 20038: note = Any amino acid
  VARIANT 20044..20048: note = Any amino acid
  VARIANT 20050..20051: note = Any amino acid
  VARIANT 20062: note = Any amino acid
  VARIANT 20069: note = Any amino acid
  VARIANT 20071: note = Any amino acid
  VARIANT 20075: note = Any amino acid
  VARIANT 20079: note = Any amino acid
  VARIANT 20088: note = Any amino acid
  VARIANT 20091: note = Any amino acid
  VARIANT 20094: note = Any amino acid
  VARIANT 20100: note = Any amino acid
  VARIANT 20105: note = Any amino acid
  VARIANT 20107..20109: note = Any amino acid
  VARIANT 20112: note = Any amino acid
  VARIANT 20116..20117: note = Any amino acid
  VARIANT 20119: note = Any amino acid
  VARIANT 20126: note = Any amino acid
  VARIANT 20129..20130: note = Any amino acid
  VARIANT 20132: note = Any amino acid
  VARIANT 20142: note = Any amino acid
  VARIANT 20272..20273: note = Any amino acid
  VARIANT 20275: note = Any amino acid
  VARIANT 20282: note = Any amino acid
  VARIANT 20285..20286: note = Any amino acid
  VARIANT 20288: note = Any amino acid
  VARIANT 20298: note = Any amino acid
  VARIANT 20308..20309: note = Any amino acid
  VARIANT 20311: note = Any amino acid
  VARIANT 20323..20324: note = Any amino acid
  VARIANT 20333: note = Any amino acid
  VARIANT 20335..20336: note = Any amino acid
  VARIANT 20806..20808: note = Any amino acid
  VARIANT 20812: note = Any amino acid
  VARIANT 20816: note = Any amino acid
  VARIANT 20818: note = Any amino acid
  VARIANT 20824..20828: note = Any amino acid
  VARIANT 20830..20831: note = Any amino acid
MLKPSGLPGSSSPTRSLMTGSRSTKATPEMDSGLTGATLSPKTSTGAIVVTEHTLPFTSPDKTLASPTSSVVGRTTQSLGVMSSALPESTSRGMTHSEQRTSPSLSPQVNGTPSRNYPATSMVSGLSSPRTRTSSTEGNFTKEASTYTLTVETTSGPVTEKYTVPTETSTTEGDSTETPWDTRYIPVKITSPMKTFADSTASKENAPVSMTPAETTVTDSHTPGRTNPSFGTLYSSFLDLSPKGTPNSRGETSLELILSTTGYPFSSPEPGSAGHSRISTSAPLSSSASVLDNKISETSIFSGQSLTSPLSPGVPEARASTMPNSAIPFSMTLSNAETSAERVRSTISSLGTPSISTKQTAETILTFHAFAETMDIPSTHIAKTLASEWLGSPGTLGGTSTSALTTTSPSTTLVSEETNTHHSTSGKETEGTLNTSMTPLETSAPGEESEMTATLVPTLGFTTLDSKIRSPSQVSSSHPTRELRTTGSTSGRQSSSTAAHGSSDILRATTSSTSKASSWTSESTAQQFSEPQHTQWVETSPSMKTERPPASTSVAAPITTSVPSVVSGFTTLKTSSTKGIWLEETSADTLIGESTAGPTTHQFAVPTGISMTGGSSTRGSQGTTHLLTRATASSETSADLTLATNGVPVSVSPAVSKTAAGSSPPGGTKPSYTMVSSVIPETSSLQSSAFREGTSLGLTPLNTRHPFSSPEPDSAGHTKISTSIPLLSSASVLEDKVSATSTFSHHKATSSITTGTPEISTKTKPSSAVLSSMTLSNAATSPERVRNATSPLTHPSPSGEETAGSVLTLSTSAETTDSPNIHPTGTLTSESSESPSTLSLPSVSGVKTTFSSSTPSTHLFTSGEETEETSNPSVSQPETSVSRVRTTLASTSVPTPVFPTMDTWPTRSAQFSSSHLVSELRATSSTSVTNSTGSALPKISHLTGTATMSQTNRDTFNDSAAPQSTTWPETSPRFKTGLPSATTTVSTSATSLSATVMVSKFTSPATSSMEATSIREPSTTILTTETTNGPGSMAVASTNIPIGKGYITEGRLDTSHLPIGTTASSETSMDFTMAKESVSMSVSPSQSMDAAGSSTPGRTSQFVDTFSDDVYHLTSREITIPRDGTSSALTPQMTATHPPSPDPGSARSTWLGILSSSPSSPTPKVTMSSTFSTQRVTTSMIMDTVETSRWNMPNLPSTTSLTPSNIPTSGAIGKSTLVPLDTPSPATSLEASEGGLPTLSTYPESTNTPSIHLGAHASSESPSTIKLTMASVVKPGSYTPLTFPSIETHIHVSTARMAYSSGSSPEMTAPGETNTGSTWDPTTYITTTDPKDTSSAQVSTPHSVRTLRTTENHPKTESATPAAYSGSPKISSSPNLTSPATKAWTITDTTEHSTQLHYTKLAEKSSGFETQSAPGPVSVVIPTSPTIGSSTLELTSDVPGEPLVLAPSEQTTITLPMATWLSTSLTEEMASTDLDISSPSSPMSTFAIFPPMSTPSHELSKSEADTSAIRNTDSTTLDQHLGIRSLGRTGDLTTVPITPLTTTWTSVIEHSTQAQDTLSATMSPTHVTQSLKDQTSIPASASPSHLTEVYPELGTQGRSSSEATTFWKPSTDTLSREIETGPTNIQSTPPMDNTTTGSSSSGVTLGIAHLPIGTSSPAETSTNMALERRSSTATVSMAGTMGLLVTSAPGRSISQSLGRVSSVLSESTTEGVTDSSKGSSPRLNTQGNTALSSSLEPSYAEGSQMSTSIPLTSSPTTPDVEFIGGSTFWTKEVTTVMTSDISKSSARTESSSATLMSTALGSTENTGKEKLRTASMDLPSPTPSMEVTPWISLTLSNAPNTTDSLDLSHGVHTSSAGTLATDRSLNTGVTRASRLENGSDTSSKSLSMGNSTHTSMTDTEKSEVSSSIHPRPETSAPGAETTLTSTPGNRAISLTLPFSSIPVEEVISTGITSGPDINSAPMTHSPITPPTIVWTSTGTIEQSTQPLHAVSSEKVSVQTQSTPYVNSVAVSASPTHENSVSSGSSTSSPYSSASLESLDSTISRRNAITSWLWDLTTSLPTTTWPSTSLSEALSSGHSGVSNPSSTTTEFPLFSAASTSAAKQRNPETETHGPQNTAASTLNTDASSVTGLSETPVGASISSEVPLPMAITSRSDVSGLTSESTANPSLGTASSAGTKLTRTISLPTSESLVSFRMNKDPWTVSIPLGSHPTTNTETSIPVNSAGPPGLSTVASDVIDTPSDGAESIPTVSFSPSPDTEVTTISHFPEKTTHSFRTISSLTHELTSRVTPIPGDWMSSAMSTKPTGASPSITLGERRTITSAAPTTSPIVLTASFTETSTVSLDNETTVKTSDILDARKTNELPSDSSSSSDLINTSIASSTMDVTKTASISPTSISGMTASSSPSLFSSDRPQVPTSTTETNTATSPSVSSNTYSLDGGSNVGGTPSTLPPFTITHPVETSSALLAWSRPVRTFSTMVSTDTASGENPTSSNSVVTSVPAPGTWASVGSTTDLPAMGFLKTSPAGEAHSLLASTIEPATAFTPHLSAAVVTGSSATSEASLLTTSESKAIHSSPQTPTTPTSGANWETSATPESLLVVTETSDTTLTSKILVTDTILFSTVSTPPSKFPSTGTLSGASFPTLLPDTPAIPLTATEPTSSLATSFDSTPLVTIASDSLGTVPETTLTMSETSNGDALVLKTVSNPDRSIPGITIQGVTESPLHPSSTSPSKIVAPRNTTYEGSITVALSTLPAGTTGSLVFSQSSENSETTALVDSSAGLERASVMPLTTGSQGMASSGGIRSGSTHSTGTKTFSSLPLTMNPGEVTAMSEITTNRLTATQSTAPKGIPVKPTSAESGLLTPVSASSSPSKAFASLTTAPPSTWGIPQSTLTFEFSEVPSLDTKSASLPTPGQSLNTIPDSDASTASSSLSKSPEKNPRARMMTSTKAISASSFQSTGFTETPEGSASPSMAGHEPRVPTSGTGDPRYASESMSYPDPSKASSAMTSTSLASKLTTLFSTGQAARSGSSSSPISLSTEKETSFLSPTASTSRKTSLFLGPSMARQPNILVHLQTSALTLSPTSTLNMSQEEPPELTSSQTIAEEEGTTAETQTLTFTPSETPTSLLPVSSPTEPTARRKSSPETWASSISVPAKTSLVETTDGTLVTTIKMSSQAAQGNSTWPAPAEETGTSPAGTSPGSPEVSTTLKIMSSKEPSISPEIRSTVRNSPWKTPETTVPMETTVEPVTLQSTALGSGSTSISHLPTGTTSPTKSPTENMLATERVSLSPSPPEAWTNLYSGTPGGTRQSLATMSSVSLESPTARSITGTGQQSSPELVSKTTGMEFSMWHGSTGGTTGDTHVSLSTSSNILEDPVTSPNSVSSLTDKSKHKTETWVSTTAIPSTVLNNKIMAAEQQTSRSVDEAYSSTSSWSDQTSGSDITLGASPDVTNTLYITSTAQTTSLVSLPSGDQGITSLTNPSGGKTSSASSVTSPSIGLETLRANVSAVKSDIAPTAGHLSQTSSPAEVSILDVTTAPTPGISTTITTMGTNSISTTTPNPEVGMSTMDSTPATERRTTSTEHPSTWSSTAASDSWTVTDMTSNLKVARSPGTISTMHTTSFLASSTELDSMSTPHGRITVIGTSLVTPSSDASAVKTETSTSERTLSPSDTTASTPISTFSRVQRMSISVPDILSTSWTPSSTEAEDVPVSMVSTDHASTKTDPNTPLSTFLFDSLSTLDWDTGRSLSSATATTSAPQGATTPQELTLETMISPATSQLPFSIGHITSAVTPAAMARSSGVTFSRPDPTSKKAEQTSTQLPTTTSAHPGQVPRSAATTLDVIPHTAKTPDATFQRQGQTALTTEARATSDSWNEKEKSTPSAPWITEMMNSVSEDTIKEVTSSSSVLKDPEYAGHKLGIWDDFIPKFGKAAHMRELPLLSPPQDKEAIHPSTNTVETTGWVTSSEHASHSTIPAHSASSKLTSPVVTTSTREQAIVSMSTTTWPESTRARTEPNSFLTIELRDVSPYMDTSSTTQTSIISSPGSTAITKGPRTEITSSKRISSSFLAQSMRSSDSPSEAITRLSNFPAMTESGGMILAMQTSPPGATSLSAPTLDTSATASWTGTPLATTQRFTYSEKTTLFSKGPEDTSQPSPPSVEETSSSSSLVPIHATTSPSNILLTSQGHSPSSTPPVTSVFLSETSGLGKTTDMSRISLEPGTSLPPNLSSTAGEALSTYEASRDTKAIHHSADTAVTNMEATSSEYSPIPGHTKPSKATSPLVTSHIMGDITSSTSVFGSSETTEIETVSSVNQGLQERSTSQVASSATETSTVITHVSSGDATTHVTKTQATFSSGTSISSPHQFITSTNTFTDVSTNPSTSLIMTESSGVTITTQTGPTGAATQGPYLLDTSTMPYLTETPLAVTPDFMQSEKTTLISKGPKDVTWTSPPSVAETSYPSSLTPFLVTTIPPATSTLQGQHTSSPVSATSVLTSGLVKTTDMLNTSMEPVTNSPQNLNNPSNEILATLAATTDIETIHPSINKAVTNMGTASSAHVLHSTLPVSSEPSTATSPMVPASSMGDALASISIPGSETTDIEGEPTSSLTAGRKENSTLQEMNSTTESNIILSNVSVGAITEATKMEVPSFDATFIPTPAQSTKFPDIFSVASSRLSNSPPMTISTHMTTTQTGSSGATSKIPLALDTSTLETSAGTPSVVTEGFAHSKITTAMNNDVKDVSQTNPPFQDEASSPSSQAPVLVTTLPSSVAFTPQWHSTSSPVSMSSVLTSSLVKTAGKVDTSLETVTSSPQSMSNTLDDISVTSAATTDIETTHPSINTVVTNVGTTGSAFESHSTVSAYPEPSKVTSPNVTTSTMEDTTISRSIPKSSKTTRTETETTSSLTPKLRETSISQEITSSTETSTVPYKELTGATTEVSRTDVTSSSSTSFPGPDQSTVSLDISTETNTRLSTSPIMTESAEITITTQTGPHGATSQDTFTMDPSNTTPQAGIHSAMTHGFSQLDVTTLMSRIPQDVSWTSPPSVDKTSSPSSFLSSPAMTTPSLISSTLPEDKLSSPMTSLLTSGLVKITDILRTRLEPVTSSLPNFSSTSDKILATSKDSKDTKEIFPSINTEETNVKANNSGHESHSPALADSETPKATTQMVITTTVGDPAPSTSMPVHGSSETTNIKREPTYFLTPRLRETSTSQESSFPTDTSFLLSKVPTGTITEVSSTGVNSSSKISTPDHDKSTVPPDTFTGEIPRVFTSSIKTKSAEMTITTQASPPESASHSTLPLDTSTTLSQGGTHSTVTQGFPYSEVTTLMGMGPGNVSWMTTPPVEETSSVSSLMSSPAMTSPSPVSSTSPQSIPSSPLPVTALPTSVLVTTTDVLGTTSPESVTSSPPNLSSITHERPATYKDTAHTEAAMHHSTNTAVTNVGTSGSGHKSQSSVLADSETSKATPLMSTTSTLGDTSVSTSTPNISQTNQIQTEPTASLSPRLRESSTSEKTSSTTETNTAFSYVPTGAITQASRTEISSSRTSISDLDRPTIAPDISTGMITRLFTSPIMTKSAEMTVTTQTTTPGATSQGILPWDTSTTLFQGGTHSTVSQGFPHSEITTLRSRTPGDVSWMTTPPVEETSSGFSLMSPSMTSPSPVSSTSPESIPSSPLPVTALLTSVLVTTTNVLGTTSPETVTSSPPNLSSPTQERLTTYKDTAHTEAMHASMHTNTAVANVGTSISGHESQSSVPADSHTSKATSPMGITFAMGDTSVSTSTPAFFETRIQTESTSSLIPGLRDTRTSEEINTVTETSTVLSEVPTTTTTEVSRTEVITSSRTTISGPDHSKMSPYISTETITRLSTFPFVTGSTEMAITNQTGPIGTISQATLTLDTSSTASWEGTHSPVTQRFPHSEETTTMSRSTKGVSWQSPPSVEETSSPSSPVPLPAITSHSSLYSAVSGSSPTSALPVTSLLTSGRRKTIDMLDTHSELVTSSLPSASSFSGEILTSEASTNTETIHFSENTAETNMGTTNSMHKLHSSVSIHSQPSGHTPPKVTGSMMEDAIVSTSTPGSPETKNVDRDSTSPLTPELKEDSTALVMNSTTESNTVFSSVSLDAATEVSRAEVTYYDPTFMPASAQSTKSPDISPEASSSHSNSPPLTISTHKTIATQTGPSGVTSLGQLTLDTSTIATSAGTPSARTQDFVDSETTSVMNNDLNDVLKTSPFSAEEANSLSSQAPLLVTTSPSPVTSTLQEHSTSSLVSVTSVPTPTLAKITDMDTNLEPVTRSPQNLRNTLATSEATTDTHTMHPSINTAMANVGTTSSPNEFYFTVSPDSDPYKATSAVVITSTSGDSIVSTSMPRSSAMKKIESETTFSLIFRLRETSTSQKIGSSSDTSTVFDKAFTAATTEVSRTELTSSSRTSIQGTEKPTMSPDTSTRSVTMLSTFAGLTKSEERTIATQTGPHRATSQGTLTWDTSITTSQAGTHSAMTHGFSQLDLSTLTSRVPEYISGTSPPSVEKTSSSSSLLSLPAITSPSPVPTTLPESRPSSPVHLTSLPTSGLVKTTDMLASVASLPPNLGSTSHKIPTTSEDIKDTEKMYPSTNIAVTNVGTTTSEKESYSSVPAYSEPPKVTSPMVTSFNIRDTIVSTSMPGSSEITRIEMESTFSVAHGLKGTSTSQDPIVSTEKSAVLHKLTTGATETSRTEVASSRRTSIPGPDHSTESPDISTEVIPSLPISLGITESSNMTIITRTGPPLGSTSQGTFTLDTPTTSSRAGTHSMATQEFPHSEMTTVMNKDPEILSWTIPPSIEKTSFSSSLMPSPAMTSPPVSSTLPKTIHTTPSPMTSLLTPSLVMTTDTLGTSPEPTTSSPPNLSSTSHVILTTDEDTTAIEAMHPSTSTAATNVETTCSGHGSQSSVLTDSEKTKATAPMDTTSTMGHTTVSTSMSVSSETTKIKRESTYSLTPGLRETSISQNASFSTDTSIVLSEVPTGTTAEVSRTEVTSSGRTSIPGPSQSTVLPEISTRTMTRLFASPTMTESAEMTIPTQTGPSGSTSQDTLTLDTSTTKSQAKTHSTLTQRFPHSEMTTLMSRGPGDMSWQSSPSLENPSSLPSLLSLPATTSPPPISSTLPVTISSSPLPVTSLLTSSPVTTTDMLHTSPELVTSSPPKLSHTSDERLTTGKDTTNTEAVHPSTNTAASNVEIPSFGHESPSSALADSETSKATSPMFITSTQEDTTVAISTPHFLETSRIQKESISSLSPKLRETGSSVETSSAIETSAVLSEVSIGATTEISRTEVTSSSRTSISGSAESTMLPEISTTRKIIKFPTSPILAESSEMTIKTQTSPPGSTSESTFTLDTSTTPSLVITHSTMTQRLPHSEITTLVSRGAGDVPRPSSLPVEETSPPSSQLSLSAMISPSPVSSTLPASSHSSSASVTSPLTPGQVKTTEVLDASAEPETSSPPSLSSTSVEILATSEVTTDTEKIHPFPNTAVTKVGTSSSGHESPSSVLPDSETTKATSAMGTISIMGDTSVSTLTPALSNTRKIQSEPASSLTTRLRETSTSEETSLATEANTVLSKVSTGATTEVSRTEAISFSRTSMSGPEQSTMSQDISIGTIPRISASSVLTESAKMTITTQTGPSESTLESTLNLNTATTPSWVETHSIVIQGFPHPEMTTSMGRGPGGVSWPSPPFVKETSPPSSPLSLPAVTSPHPVSTTFLAHIPPSPLPVTSLLTSGPATTTDILGTSTEPGTSSSSSLSTTSHERLTTYKDTAHTEAVHPSTNTGGTNVATTSSGYKSQSSVLADSSPMCTTSTMGDTSVLTSTPAFLETRRIQTELASSLTPGLRESSGSEGTSSGTKMSTVLSKVPTGATTEISKEDVTSIPGPAQSTISPDISTRTVSWFSTSPVMTESAEITMNTHTSPLGATTQGTSTLATSSTTSLTMTHSTISQGFSHSQMSTLMRRGPEDVSWMSPPLLEKTRPSFSLMSSPATTSPSPVSSTLPESISSSPLPVTSLLTSGLAKTTDMLHKSSEPVTNSPANLSSTSVEILATSEVTTDTEKTHPSSNRTVTDVGTSSSGHESTSFVLADSQTSKVTSPMVITSTMEDTSVSTSTPGFFETSRIQTEPTSSLTLGLRKTSSSEGTSLATEMSTVLSGVPTGATAEVSRTEVTSSSRTSISGFAQLTVSPETSTETITRLPTSSIMTESAEMMIKTQTDPPGSTPESTHTVDISTTPNWVETHSTVTQRFSHSEMTTLVSRSPGDMLWPSQSSVEETSSASSLLSLPATTSPSPVSSTLVEDFPSASLPVTSLLTPGLVITTDRMGISREPGTSSTSNLSSTSHERLTTLEDTVDTEDMQPSTHTAVTNVRTSISGHESQSSVLSDSETPKATSPMGTTYTMGETSVSISTSDFFETSRIQIEPTSSLTSGLRETSSSERISSATEGSTVLSEVPSGATTEVSRTEVISSRGTSMSGPDQFTISPDISTEAITRLSTSPIMTESAESAITIETGSPGATSEGTLTLDTSTTTFWSGTHSTASPGFSHSEMTTLMSRTPGDVPWPSLPSVEEASSVSSSLSSPAMTSTSFFSALPESISSSPHPVTALLTLGPVKTTDMLRTSSEPETSSPPNLSSTSAEILATSEVTKDREKIHPSSNTPVVNVGTVIYKHLSPSSVLADLVTTKPTSPMATTSTLGNTSVSTSTPAFPETMMTQPTSSLTSGLREISTSQETSSATERSASLSGMPTGATTKVSRTEALSLGRTSTPGPAQSTISPEISTETITRISTPLTTTGSAEMTITPKTGHSGASSQGTFTLDTSSRASWPGTHSAATHRSPHSGMTTPMSRGPEDVSWPSRPSVEKTSPPSSLVSLSAVTSPSPLYSTPSESSHSSPLRVTSLFTPVMMKTTDMLDTSLEPVTTSPPSMNITSDESLATSKATMETEAIQLSENTAVTQMGTISARQEFYSSYPGLPEPSKVTSPVVTSSTIKDIVSTTIPASSEITRIEMESTSTLTPTPRETSTSQEIHSATKPSTVPYKALTSATIEDSMTQVMSSSRGPSPDQSTMSQDISSEVITRLSTSPIKAESTEMTITTQTGSPGATSRGTLTLDTSTTFMSGTHSTASQGFSHSQMTALMSRTPGDVPWLSHPSVEEASSASFSLSSPVMTSSSPVSSTLPDSIHSSSLPVTSLLTSGLVKTTELLGTSSEPETSSPPNLSSTSAEILATTEVTTDTEKLEMTNVVTSGYTHESPSSVLADSVTTKATSSMGITYPTGDTNVLTSTPAFSDTSRIQTKSKLSLTPGLMETSISEETSSATEKSTVLSSVPTGATTEVSRTEAISSSRTSIPGPAQSTMSSDTSMETITRISTPLTRKESTDMAITPKTGPSGATSQGTFTLDSSSTASWPGTHSATTQRFPQSVVTTPMSRGPEDVSWPSPLSVEKNSPPSSLVSSSSVTSPSPLYSTPSGSSHSSPVPVTSLFTSIMMKATDMLDASLEPETTSAPNMNITSDESLATSKATTETEAIHVFENTAASHVETTSATEELYSSSPGFSEPTKVISPVVTSSSIRDNMVSTTMPGSSGITRIEIESMSSLTPGLRETRTSQDITSSTETSTVLYKMSSGATPEVSRTEVMPSSRTSIPGPAQSTMSLDISDEVVTRLSTSPIMTESAEITITTQTGYSLATSQVTLPLGTSMTFLSGTHSTMSQGLSHSEMTNLMSRGPESLSWTSPRFVETTRSSSSLTSLPLTTSLSPVSSTLLDSSPSSPLPVTSLILPGLVKTTEVLDTSSEPKTSSSPNLSSTSVEIPATSEIMTDTEKIHPSSNTAVAKVRTSSSVHESHSSVLADSETTITIPSMGITSAVDDTTVFTSNPAFSETRRIPTEPTFSLTPGFRETSTSEETTSITETSAVLYGVPTSATTEVSMTEIMSSNRTHIPDSDQSTMSPDIITEVITRLSSSSMMSESTQMTITTQKSSPGATAQSTLTLATTTAPLARTHSTVPPRFLHSEMTTLMSRSPENPSWKSSPFVEKTSSSSSLLSLPVTTSPSVSSTLPQSIPSSSFSVTSLLTPGMVKTTDTSTEPGTSLSPNLSGTSVEILAASEVTTDTEKIHPSSSMAVTNVGTTSSGHELYSSVSIHSEPSKATYPVGTPSSMAETSISTSMPANFETTGFEAEPFSHLTSGFRKTNMSLDTSSVTPTNTPSSPGSTHLLQSSKTDFTSSAKTSSPDWPPASQYTEIPVDIITPFNASPSITESTGITSFPESRFTMSVTESTHHLSTDLLPSAETISTGTVMPSLSEAMTSFATTGVPRAISGSGSPFSRTESGPGDATLSTIAESLPSSTPVPFSSSTFTTTDSSTIPALHEITSSSATPYRVDTSLGTESSTTEGRLVMVSTLDTSSQPGRTSSTPILDTRMTESVELGTVTSAYQVPSLSTRLTRTDGIMEHITKIPNEAAHRGTIRPVKGPQTSTSPASPKGLHTGGTKRMETTTTALKTTTTALKTTSRATLTTSVYTPTLGTLTPLNASRQMASTILTEMMITTPYVFPDVPETTSSLATSLGAETSTALPRTTPSVLNRESETTASLVSRSGAERSPVIQTLDVSSSEPDTTASWVIHPAETIPTVSKTTPNFFHSELDTVSSTATSHGADVSSAIPTNISPSELDALTPLVTISGTDTSTTFPTLTKSPHETETRTTWLTHPAETSSTIPRTIPNFSHHESDATPSIATSPGAETSSAIPIMTVSPGAEDLVTSQVTSSGTDRNMTIPTLTLSPGEPKTIASLVTHPEAQTSSAIPTSTISPAVSRLVTSMVTSLAAKTSTTNRALTNSPGEPATTVSLVTHPAQTSPTVPWTTSIFFHSKSDTTPSMTTSHGAESSSAVPTPTVSTEVPGVVTPLVTSSRAVISTTIPILTLSPGEPETTPSMATSHGEEASSAIPTPTVSPGVPGVVTSLVTSSRAVTSTTIPILTFSLGEPETTPSMATSHGTEAGSAVPTVLPEVPGMVTSLVASSRAVTSTTLPTLTLSPGEPETTPSMATSHGAEASSTVPTVSPEVPGVVTSLVTSSSGVNSTSIPTLILSPGELETTPSMATSHGAEASSAVPTPTVSPGVSGVVTPLVTSSRAVTSTTIPILTLSSSEPETTPSMATSHGVEASSAVLTVSPEVPGMVTSLVTSSRAVTSTTIPTLTISSDEPETTTSLVTHSEAKMISAIPTLAVSPTVQGLVTSLVTSSGSETSAFSNLTVASSQPETIDSWVAHPGTEASSVVPTLTVSTGEPFTNISLVTHPAESSSTLPRTTSRFSHSELDTMPSTVTSPEAESSSAISTTISPGIPGVLTSLVTSSGRDISATFPTVPESPHESEATASWVTHPAVTSTTVPRTTPNYSHSEPDTTPSIATSPGAEATSDFPTITVSPDVPDMVTSQVTSSGTDTSITIPTLTLSSGEPETTTSFITYSETHTSSAIPTLPVSPGASKMLTSLVISSGTDSTTTFPTLTETPYEPETTAIQLIHPAETNTMVPKTTPKFSHSKSDTTLPVAITSPGPEASSAVSTTTISPDMSDLVTSLVPSSGTDTSTTFPTLSETPYEPETTVTWLTHPAETSTTVSGTIPNFSHRGSDTAPSMVTSPGVDTRSGVPTTTIPPSIPGVVTSQVTSSATDTSTAIPTLTPSPGEPETTASSATHPGTQTGFTVPIRTVPSSEPDTMASWVTHPPQTSTPVSRTTSSFSHSSPDATPVMATSPRTEASSAVLTTISPGAPEMVTSQITSSGAATSTTVPTLTHSPGMPETTALLSTHPRTGTSKTFPASTVFPQVSETTASLTIRPGAETSTALPTQTTSSLFTLLVTGTSRVDLSPTASPGVSAKTAPLSTHPGTETSTMIPTSTLSLGLLETTGLLATSSSAETSTSTLTLTVSPAVSGLSSASITTDKPQTVTSWNTETSPSVTSVGPPEFSRTVTGTTMTLIPSEMPTPPKTSHGEGVSPTTILRTTMVEATNLATTGSSPTVAKTTTTFNTLAGSLFTPLTTPGMSTLASESVTSRTSYNHRSWISTTSSYNRRYWTPATSTPVTSTFSPGISTSSIPSSTAATVPFMVPFTLNFTITNLQYEEDMRHPGSRKFNATERELQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTAAGPLLMPFTLNFTITNLQYEEDMRRTGSRKFNTMESVLQGLLKPLFKNTSVGPLYSGCRLTLLRPEKDGAATGVDAICTHRLDPKSPGLNREQLYWELSKLTNDIEELGPYTLDRNSLYVNGFTHQSSVSTTSTPGTSTVDLRTSGTPSSLSSPTIMAAGPLLVPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLDREQLYWELSQLTNGIKELGPYTLDRNSLYVNGFTHWIPVPTSSTPGTSTVDLGSGTPSSLPSPTAAGPLLVPFTLNFTITNLQYEEDMHHPGSRKFNTTERVLQGLLGPMFKNTSVGLLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMRHPGSRKFNTTERVLQGLLKPLFKSTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATGMDAICSHRLDPKSPGLNREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNTTERVLQGLLSPIFKNSSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTSLRPEKDGAATGMDAVCLYHPNPKRPGLDREQLYWELSQLTHNITELGPYSLDRDSLYVNGFTHQNSVPTTSTPGTSTVYWATTGTPSSFPGHTEPGPLLIPFTFNFTITNLHYEENMQHPGSRKFNTTERVLQGLLKPLFKNTSVGPLYSGCRLTLLRPEKHEAATGVDTICTHRVDPIGPGLDRERLYWELSQLTNSITELGPYTLDRDSLYVNGFNPRSSVPTTSTPGTSTVHLATSGTPSSLPGHTAPVPLLIPFTLNFTITNLHYEENMQHPGSRKFNTTERVLQGLLKPLFKNTSVGPLYSGCRLTLLRPEKHEAATGVDTICTHRVDPIGPGLXXEXLYWELSXLTXXIXELGPYTLDRXSLYVNGFTHXXSXPTTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGAATGMDAICSHRLDPKSPGLDREQLYWELSQLTHGIKELGPYTLDRNSLYVNGFTHRSSVAPTSTPGTSTVDLGTSGTPSSLPSPTTAVPLLVPFTLNFTITNLQYGEDMRHPGSRKFNTTERVLQGLLGPLFKNSSVGPLYSGCRLISLRSEKDGAATGVDAICTHHLNPQSPGLDREQLYWQLSQMTNGIKELGPYTLDRNSLYVNGFTHRSSGLTTSTPWTSTVDLGTSGTPSPVPSPTTAGPLLVPFTLNFTITNLQYEEDMHRPGSRKFNATERVLQGLLSPIFKNSSVGPLYSGCRLTSLRPEKDGAATGMDAVCLYHPNPKRPGLDREQLYWELSQLTHNITELGPYSLDRDSLYVNGFTHQSSMTTTRTPDTSTMHLATSRTPASLSGPTTASPLLVLFTINCTITNLQYEEDMRRTGSRKFNTMESVLQGLLKPLFKNTSVGPLYSGCRLTLLRPKKDGAATGVDAICTHRLDPKSPGLNREQLYWELSKLTNDIEELGPYTLDRNSLYVNGFTHQSSVSTTSTPGTSTVDLRTSGTPSSLSSPTIMXXXPLLXPFTXNXTITNLXXXXXMXXPGSRKFNTTERVLQGLLRPLFKNTSVSSLYSGCRLTLLRPEKDGAATRVDAACTYRPDPKSPGLDREQLYWELSQLTHSITELGPYTLDRVSLYVNGFNPRSSVPTTSTPGTSTVHLATSGTPSSLPGHTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSGLTTSTPWTSTVDLGTSGTPSPVPSPTTAGPLLVPFTLNFTITNLQYEEDMHRPGSRRFNTTERVLQGLLTPLFKNTSVGPLYSGCRLTLLRPEKQEAATGVDTICTHRVDPIGPGLDRERLYWELSQLTNSITELGPYTLDRDSLYVNGFNPWSSVPTTSTPGTSTVHLATSGTPSSLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTHRSSVPTTSIPGTSAVHLETSGTPASLPGHTAPGPLLVPFTLNFTITNLQYEEDMRHPGSRKFSTTERVLQGLLKPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTTLRPKKDGAATKVDAICTYRPDPKSPGLDREQLYWELSQLTHSITELGPYTQDRDSLYVNGFTHRSSVPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRTSVPTTSTPGTSTVDLGTSGTPFSLPSPAXXXPLLXPFTXNXTITNLXXXXXMXXPGSRKFNTTERVLQTLLGPMFKNTSVGLLYSGCRLTLLRSEKDGAATGVDAICTHRLDPKSPGVDREQLYWELSQLTNGIKELGPYTLDRNSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTLLRSEKDGAATGVDAICTHRLDPKSPGVDREQLYWELSQLTNGIKELGPYTLDRNSLYVNGFTHQTSAPNTSTPGTSTVDLGTSGTPSSLPSPTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLKYEEDMHCPGSRKFNTTERVLQSLLGPMFKNTSVGPLYSGCRLTSLRSEKDGAATGVDAICTHRVDPKSPGVDREQLYWELSQLTNGIKELGPYTLDRNSLYVNGFTHQTSAPNTSTPGTSTVXXGTSGTPSSXPXXTSAGPLLVPFTLNFTITNLQYEEDMHHPGSRKFNTTERVLQGLLGPMFKNTSVGLLYSGCRLTLLRPEKNGATTGMDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLKPLFRNSSLEYLYSGCRLASLRPEKDSSAMAVDAICTHRPDPEDLGLDRERLYWELSNLTNGIQELGPYTLDRNSLYVNGFTHRSSMPTTSTPGTSTVDVGTSGTPSSSPSPTTAGPLLIPFTLNFTITNLQYGEDMGHPGSRKFNTTERVLQGLLGPIFKNTSVGPLYSGCRLTSLRSEKDGAATGVDAICIHHLDPKSPGLNRERLYWELSQLTNGIKELGPYTLDRNSLYVNGFTHRTSVPTTSTPGTSTVDLGTSGTPFSLPSPATAGPLLVLFTLNFTITNLKYEEDMHRPGSRKFNTTERVLQTLLGPMFKNTSVGLLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTERVLQGLLRPVFKNTSVGPLYSGCRLTLLRPKKDGAATKVDAICTYRPDPKSPGLDREQLYWELSQLTHSITELGPYTQDRDSLYVNGFTHRSSVPTTSIPGTSAVHLETTGTPSSFPGHTEPGPLLIPFTFNFTITNLRYEENMQHPGSRKFNTTERVLQGLLTPLFKNTSVGPLYSGCRLTLLRPEKQEAATGVDTICTHRVDPIGPGLDRERLYWELSQLTNSITELGPYTLDRDSLYVDGFNPWSSVPTTSTPGTSTVHLATSGTPSPLPGHTAPVPLLIPFTLNFTITDLHYEENMQHPGSRKFNTTERVLQGLLKPLFKSTSVGPLYSGCRLTLLRPEKHGAATGVDAICTLRLDPTGPGLDRERLYWELSQLTNSITELGPYTLDRDSLYVNGFNPWSSVPTTSTPGTSTVHLATSGTPSSLPGHTTAGPLLVPFTLNFTITNLKYEEDMHCPGSRKFNTTERVLQSLHGPMFKNTSVGPLYSGCRLTLLRSEKDGAATGVDAICTHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTNSITELGPYTLDRDSLYVNGFTHRSSMPTTSIPGTSAVHLETSGTPASLPGHTAPGPLLVPFTLNFTITNLQYEEDMRHPGSRKFNTTERVLQGLLKPLFKSTSVGPLYSGCRLTLLRPEKRGAATGVDTICTHRLDPLNPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFHPRSSVPTTSTPGTSTVHLATSGTPSSLPGHTAPVPLLIPFTLNFTITNLHYEENMQHPGSRKFNTTERVLQGLLGPMFKNTSVGLLYSGCRLTLLRPEKNGAATGMDAICSHRLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQNSVPTTSTPGTSTVYWATTGTPSSFPGHTEPGPLLIPFTFNFTITNLHYEENMQHPGSRKFNTTERVLQGLLTPLFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSSVPTTSSPGTSTVHLATSGTPSSLPGHTAPVPLLIPFTLNFTITNLHYEENMQHPGSRKFNTTERVLQGLLKPLFKSTSVGPLYSGCRLTLLRPEKHGAATGVDAICTLRLDPTGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRTSVPTTSTPGTSTVHLATSGTPSSLPGHTAPVPLLIPFTLNFTITNLQYEEDMHRPGSRKFNTTERVLQGLLSPIFKNSSVGPLYSGCRLTSLRPEKDGAATGMDAVCLYHPNPKRPGLDREQLYCELSQLTHNITELGPYSLDRDSLYVNGFTHQNSVPTTSTPGTSTVYWATTGTPSSFPGHTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWSSGLTTSTPWTSTVDLGTSGTPSPVPSPTTAGPLLVPFTLNFTITNLQYEEDMHRPGSRKFNATERVLQGLLSPIFKNTSVGPLYSGCRLTLLRPEKQEAATGVDTICTHRVDPIGPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHRSFGLTTSTPWTSTVDLGTSGTPSPVPSPTTAGPLLVPFTLNFTITNLQYEEDMHRPGSRKFNTTERVLQGLLTPLFRNTSVSSLYSGCRLTLLRPEKDGAATRVDAVCTHRPDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHWIPVPTSSTPGTSTVDLGSGTPSSLPSPTTAGPLLVPFTLNFTITNLQYGEDMGHPGSRKFNTTERVLQGLLGPIFKNTSVGPLYSGCRLTSLRSEKDGAATGVDAICIHHLDPKSPGLXXEXLYWELSXLTXXIXELGPYTLDRXSLYVNGFTHXXSXPTTSTPGTSTVXXGTSGTPSSXPXXTXXXPLLXPFTXNXTITNLXXXXXMXXPGSRKFNTTEXVLQGLLXPXFKNXSVGXLYSGCRLTXLRXEKXGAATGXDAICXHXXXPKXPGLXXEXLYWELSXLTXXIXELGPYTLDRXSLYVNGFTHQTFAPNTSTPGTSTVDLGTSGTPSSLPSPTSAGPLLVPFTLNFTITNLQYEEDMHHPGSRKFNTTERVLQGLLGPMFKNTSVGLLYSGCRLTLLRPEKNGAATRVDAVCTHRPDPKSPGLXXEXLYWELSXLTXXIXELGPYTLDRXSLYVNGFTHXXSXPTTSTPGTSTVXXGTSGTPSSXPXXTAPVPLLIPFTLNFTITNLHYEENMQHPGSRKFNTTERVLQGLLKPLFKSTSVGPLYSGCRLTLLRPEKHGAATGVDAICTLRLDPTGPGLDRERLYWELSQLTNSVTELGPYTLDRDSLYVNGFTQRSSVPTTSIPGTSAVHLETSGTPASLPGHTAPGPLLVPFTLNFTITNLQYEVDMRHPGSRKFNTTERVLQGLLKPLFKSTSVGPLYSGCRLTLLRPEKRGAATGVDTICTHRLDPLNPGLDREQLYWELSKLTRGIIELGPYLLDRGSLYVNGFTHRNFVPITSTPGTSTVHLGTSETPSSLPRPIVPGPLLVPFTLNFTITNLQYEEAMRHPGSRKFNTTERVLQGLLRPLFKNTSIGPLYSSCRLTLLRPEKDKAATRVDAICTHHPDPQSPGLNREQLYWELSQLTHGITELGPYTLDRDSLYVDGFTHWSPIPTTSTPGTSIVNLGTSGIPPSLPETTXXXPLLXPFTXNXTITNLXXXXXMXXPGSRKFNTTERVLQGLLKPLFKSTSVGPLYSGCRLTLLRPEKDGVATRVDAICTHRPDPKIPGLDRQQLYWELSQLTHSITELGPYTLDRDSLYVNGFTQRSSVPTTSTPGTFTVQPETSETPSSLPGPTATGPVLLPFTLNFTITNLQYEEDMHRPGSRKFNTTERVLQGLLMPLFKNTSVSSLYSGCRLTLLRPEKDGAATRVDAVCTHRPDPKSPGLDRERLYWKLSQLTHGITELGPYTLDRHSLYVNGFTHQSSMTTTRTPDTSTMHLATSRTPASLSGPTTASPLLVLFTINFTITNLRYEENMHHPGSRKFNTTERVLQGLLRPVFKNTSVGPLYSGCRLTLLRPKKDGAATKVDAICTYRPDPKSPGLDREQLYWELSQLTHSITELGPYTLDRDSLYVNGFTQRSSVPTTSIPGTPTVDLGTSGTPVSKPGPSAASPLLVLFTLNFTITNLRYEENMQHPGSRKFNTTERVLQGLLRSLFKSTSVGPLYSGCRLTLLRPEKDGTATGVDAICTHHPDPKSPRLDREQLYWELSQLTHNITELGHYALDNDSLFVNGFTHRSSVSTTSTPGTPTVYLGASKTPASIFGPSAASHLLILFTLNFTITNLRYEENMWPGSRKFNTTERVLQGLLRPLFKNTSVGPLYSGSRLTLLRPEKDGEATGVDAICTHRPDPTGPGLDREQLYLELSQLTHSITELGPYTLDRDSLYVNGFTHRSSVPTTSTGVVSEEPFTLNFTINNLRYMADMGQPGSLKFNITDNVMKHLLSPLFQRSSLGARYTGCRVIALRSVKNGAETRVDLLCTYLQPLSGPGLPIKQVFHELSQQTHGITRLGPYSLDKDSLYLNGYNEPGLDEPPTTPKPATTFLPPLSEATTAMGYHLKTLTLNFTISNLQYSPDMGKGSATFNSTEGVLQHLLRPLFQKSSMGPFYLGCQLISLRPEKDGAATGVDTTCTYHPDPVGPGLDIQQLYWELSQLTHGVTQLGFYVLDRDSLFINGYAPQNLSIRGEYQINFHIVNWNLSNPDPTSSEYITLLRDIQDKVTTLYKGSQLHDTFRFCLVTNLTMDSVLVTVKALFSSNLDPSLVEQVFLDKTLNASFHWLGSTYQLVDIHVTEMESSVYQPTSSSSTQHFYLNFTITNLPYSQDKAQPGTTNYQRNKRNIEDALNQLFRNSSIKSYFSDCQVSTFRSVPNRHHTGVDSLCNFSPLARRVDRVAIYEEFLRMTRNGTQLQNFTLDRSSVLVDGYSPNRNEPLTGNSDLPFWAVILIGLAGLLGLITCLICGVLVTTRRRKKEGEYNVQQQCPGYYQSHLDLEDLQ

SEQ ID NO: 5 - protein, 147 aa, Homo sapiens
MASHRLLLLCLAGLVFVSEAGPTGTGESKCPLMVKVLDAVRGSPAINVAVHVFRKAADDTWEPFASGKTSESGELHGLTTEEEFVEGIYKVEIDTKSYWKALGISPFHEHAEVVFTANDSGPRRYTIAALLSPYSYSTTAVVTNPKE

SEQ ID NO: 6 - protein, 698 aa, Homo sapiens
MRLAVGALLVCAVLGLCLAVPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSIGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPGAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 7 - protein, 267 aa, Homo sapiens
MKAAVLTLAVLFLTGSQARHFWQQDEPPQSPWDRVKDLATVYVDVLKDSGRDYVSQFEGSALGKQLNLKLLDNWDSVTSTFSKLREQLGPVTQEFWDNLEKETEGLRQEMSKDLEEVKAKVQPYLDDFQKKWQEEMELYRQKVEPLRAELQEGARQKLHELQEKLSPLGEEMRDRARAHVDALRTHLAPYSDELRQRLAARLEALKENGGARLAEYHAKATEHLSTLSEKAKPALEDLRQGLLPVLESFKVSFLSALEEYTKKLNTQ

SEQ ID NO: 8 - protein, 119 aa, Homo sapiens
MSRSVALAVLALLSLSGLEAIQRTPKIQVYSRHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 9 - protein, 1863 aa, Homo sapiens
MDLSALRVEEVQNVINAMQKILECPICLELIKEPVSTKCDHIFCKFCMLKLLNQKKGPSQCPLCKNDITKRSLQESTRFSQLVEELLKIICAFQLDTGLEYANSYNFAKKENNSPEHLKDEVSIIQSMGYRNRAKRLLQSEPENPSLQETSLSVQLSNLGTVRTLRTKQRIQPQKTSVYIELGSDSSEDTVNKATYCSVGDQELLQITPQGTRDEISLDSAKKAACEFSETDVTNTEHHQPSNNDLNTTEKRAAERHPEKYQGSSVSNLHVEPCGTNTHASSLQHENSSLLLTKDRMNVEKAEFCNKSKQPGLARSQHNRWAGSKETCNDRRTPSTEKKVDLNADPLCERKEWNKQKLPCSENPRDTEDVPWITLNSSIQKVNEWFSRSDELLGSDDSHDGESESNAKVADVLDVLNEVDEYSGSSEKIDLLASDPHEALICKSERVHSKSVESNIEDKIFGKTYRKKASLPNLSHVTENLIIGAFVTEPQIIQERPLTNKLKRKRRPTSGLHPEDFIKKADLAVQKTPEMINQGTNQTEQNGQVMNITNSGHENKTKGDSIQNEKNPNPIESLEKESAFKTKAEPISSSISNMELELNIHNSKAPKKNRLRRKSSTRHIHALELVVSRNLSPPNCTELQIDSCSSSEEIKKKKYNQMPVRHSRNLQLMEGKEPATGAKKSNKPNEQTSKRHDSDTFPELKLTNAPGSFTKCSNTSELKEFVNPSLPREEKEEKLETVKVSNNAEDPKDLMLSGERVLQTERSVESSSISLVPGTDYGTQESISLLEVSTLGKAKTEPNKCVSQCAAFENPKGLIHGCSKDNRNDTEGFKYPLGHEVNHSRETSIEMEESELDAQYLQNTFKVSKRQSFAPFSNPGNAEEECATFSAHSGSLKKQSPKVTFECEQKEENQGKNESNIKPVQTVNITAGFPVVGQKDKPVDNAKCSIKGGSRFCLSSQFRGNETGLITPNKHGLLQNPYRIPPLFPIKSFVKTKCKKNLLEENFEEHSMSPEREMGNENIPSTVSTISRNNIRENVFKEASSSNINEVGSSTNEVGSSINEIGSSDENIQAELGRNRGPKLNAMLRLGVLQPEVYKQSLPGSNCKHPEIKKQEYEEVVQTVNTDFSPYLISDNLEQPMGSSHASQVCSETPDDLLDDGEIKEDTSFAENDIKESSAVFSKSVQKGELSRSPSPFTHTHLAQGYRRGAKKLESSEENLSSEDEELPCFQHLLFGKVNNIPSQSTRHSTVATECLSKNTEENLLSLKNSLNDCSNQVILAKASQEHHLSEETKCSASLFSSQCSELEDLTANTNTQDPFLIGSSKQMRHQSESQGVGLSDKELVSDDEERGTGLEENNQEEQSMDSNLGEAASGCESETSVSEDCSGLSSQSDILTTQQRDTMQHNLIKLQQEMAELEAVLEQHGSQPSNSYPSIISDSSALEDLRNPEQSTSEKAVLTSQKSSEYPISQNPEGLSADKFEVSADSSTSKNKEPGVERSSPSKCPSLDDRWYMHSCSGSLQNRNYPSQEELIKVVDVEEQQLEESGPHDLTETSYLPRQDLEGTPYLESGISLFSDDPESDPSEDRAPESARVGNIPSSTSALKVPQLKVAESAQSPAAAHTTDTAGYNAMEESVSREKPELTASTERVNKRMSMVVSGLTPEEFMLVYKFARKHHITLTNLITEETTHVVMKTDAEFVCERTLKYFLGIAGGKWVVSYFWVTQSIKERKMLNEHDFEVRGDVVNGRNHQGPKRARESQDRKIFRGLEICCYGPFTNMPTDQLEWMVQLCGASVVKELSSFTLGTGVHPIVVVQPDAWTEDNGFHAIGQMCEAPVVTREWVLDSVALYQCQELDTYLIPQIPHSHY

SEQ ID NO: 10 - protein, 3418 aa, Homo sapiens
MPIGSKERPTFFEIFKTRCNKADLGPISLNWFEELSSEAPPYNSEPAEESEHKNNNYEPNLFKTPQRKPSYNQLASTPIIFKEQGLTLPLYQSPVKELDKFKLDLGRNVPNSRHKSLRTVKTKMDQADDVSCPLLNSCLSESPVVLQCTHVTPQRDKSVVCGSLFHTPKFVKGRQTPKHISESLGAEVDPDMSWSSSLATPPTLSSTVLIVRNEEASETVFPHDTTANVKSYFSNHDESLKKNDRFIASVTDSENTNQREAASHGFGKTSGNSFKVNSCKDHIGKSMPNVLEDEVYETVVDTSEEDSFSLCFSKCRTKNLQKVRTSKTRKKIFHEANADECEKSKNQVKEKYSFVSEVEPNDTDPLDSNVAHQKPFESGSDKISKEVVPSLACEWSQLTLSGLNGAQMEKIPLLHISSCDQNISEKDLLDTENKRKKDFLTSENSLPRISSLPKSEKPLNEETVVNKRDEEQHLESHTDCILAVKQAISGTSPVASSFQGIKKSIFRIRESPKETFNASFSGHMTDPNFKKETEASESGLEIHTVCSQKEDSLCPNLIDNGSWPATTTQNSVALKNAGLISTLKKKTNKFIYAIHDETFYKGKKIPKDQKSELINCSAQFEANAFEAPLTFANADSGLLHSSVKRSCSQNDSEEPTLSLTSSFGTILRKCSRNETCSNNTVISQDLDYKEAKCNKEKLQLFITPEADSLSCLQEGQCENDPKSKKVSDIKEEVLAAACHPVQHSKVEYSDTDFQSQKSLLYDHENASTLILTPTSKDVLSNLVMISRGKESYKMSDKLKGNNYESDVELTKNIPMEKNQDVCALNENYKNVELLPPEKYMRVASPSRKVQFNQNTNLRVIQKNQEETTSISKITVNPDSEELFSDNENNFVFQVANERNNLALGNTKELHETDLTCVNEPIFKNSTMVLYGDTGDKQATQVSIKKDLVYVLAEENKNSVKQHIKMTLGQDLKSDISLNIDKIPEKNNDYMNKWAGLLGPISNHSFGGSFRTASNKEIKLSEHNIKKSKMFFKDIEEQYPTSLACVEIVNTLALDNQKKLSKPQSINTVSAHLQSSVVVSDCKNSHITPQMLFSKQDFNSNHNLTPSQKAEITELSTILEESGSQFEFTQFRKPSYILQKSTFEVPENQMTILKTTSEECRDADLHVIMNAPSIGQVDSSKQFEGTVEIKRKFAGLLKNDCNKSASGYLTDENEVGFRGFYSAHGTKLNVSTEALQKAVKLFSDIENISEETSAEVHPISLSSSKCHDSVVSMFKIENHNDKTVSEKNNKCQLILQNNIEMTTGTFVEEITENYKRNTENEDNKYTAASRNSHNLEFDGSDSSKNDTVCIHKDETDLLFTDQHNICLKLSGQFMKEGNTQIKEDLSDLTFLEVAKAQEACHGNTSNKEQLTATKTEQNIKDFETSDTFFQTASGKNISVAKESFNKIVNFFDQKPEELHNFSLNSELHSDIRKNKMDILSYEETDIVKHKILKESVPVGTGNQLVTFQGQPERDEKIKEPTLLGFHTASGKKVKIAKESLDKVKNLFDEKEQGTSEITSFSHQWAKTLKYREACKDLELACETIEITAAPKCKEMQNSLNNDKNLVSIETVVPPKLLSDNLCRQTENLKTSKSIFLKVKVHENVEKETAKSPATCYTNQSPYSVIENSALAFYTSCSRKTSVSQTSLLEAKKWLREGIFDGQPERINTADYVGNYLYENNSNSTIAENDKNHLSEKQDTYLSNSSMSNSYSYHSDEVYNDSGYLSKNKLDSGIEPVLKNVEDQKNTSFSKVISNVKDANAYPQTVNEDICVEELVTSSSPCKNKNAAIKLSISNSNNFEVGPPAFRIASGKIVCVSHETIKKVKDIFTDSFSKVIKENNENKSKICQTKIMAGCYEALDDSEDILHNSLDNDECSTHSHKVFADIQSEEILQHNQNMSGLEKVSKISPCDVSLETSDICKCSIGKLHKSVSSANTCGIFSTASGKSVQVSDASLQNARQVFSEIEDSTKQVFSKVLFKSNEHSDQLTREENTAIRTPEHLISQKGFSYNVVNSSAFSGFSTASGKQVSILESSLHKVKGVLEEFDLIRTEHSLHYSPTSRQNVSKILPRVDKRNPEHCVNSEMEKTCSKEFKLSNNLNVEGGSSENNHSIKVSPYLSQFQQDKQQLVLGTKVSLVENIHVLGKEQASPKNVKMEIGKTETFSDVPVKTNIEVCSTYSKDSENYFETEAVEIAKAFMEDDELTDSKLPSHATHSLFTCPENEEMVLSNSRIGKRRGEPLILVGEPSIKRNLLNEFDRIIENQEKSLKASKSTPDGTIKDRRLFMHHVSLEPITCVPFRTTKERQEIQNPNFTAPGQEFLSKSHLYEHLTLEKSSSNLAVSGHPFYQVSATRNEKMRHLITTGRPTKVFVPPFKTKSHFHRVEQCVRNINLEENRQKQNIDGHGSDDSKNKINDNEIHQFNKNNSNQAAAVTFTKCEEEPLDLITSLQNARDIQDMRIKKKQRQRVFPQPGSLYLAKTSTLPRISLKAAVGGQVPSACSHKQLYTYGVSKHCIKINSKNAESFQFHTEDYFGKESLWTGKGIQLADGGWLIPSNDGKAGKEEFYRALCDTPGVDPKLISRIWVYNHYRWIIWKLAAMECAFPKEFANRCLSPERVLLQLKYRYDTEIDRSRRSAIKKIMERDDTAAKTLVLCVSDIISLSANISETSSNKTSSADTQKVAIIELTDGWYAVKAQLDPPLLAVLKNGRLTVGQKIILHGAELVGSPDACTPLEAPESLMLKISANSTRPARWYTKLGFFPDPRPFPLPLSSLFSDGGNVGCVDVIIQRAYPIQWMEKTSSGLYIFRNEREEEKEAAKYVEAQQKRLEALFTKIQEEFEEHEENTTKPYLPSRALTRQQVRALQDGAELYEAVKNAADPAYLEGYFSEEQLRALNNHRQMLNDKKQAQIQLEIRKAMESAEQKEQGLSRDVTTVWKLRIVSYSKKEKDSVILSIWRPSSDLYSLLTEGKRYRIYHLATSKSKSKSERANIQLAATKKTQYQQLPVSDEILFQIYQPREPLHFSKFLDPDFQPSCSEVDLIGFVVSVVKKTGLAPFVYLSDECYNLLAIKFWIDLNEDIIKPHMLIAASNLQWRPESKSGLLTLFAGDFSVFSASPKEGHFQETFNKMKNTVENIDILCNEAENKLMHILHANDPKWSTPTKDCTSGPYTAQIIPGTGNKLLMSSPNCEIYYQSPLSLCMAKRKSVSTPVSAQMTSKSCKGEKEIDDQKNCKKRRALDFLSRLPLPPPVSPICTFVSPAAQKAFQPPRSCGTKYETPIKKKELNSPQMTPFKKFNEISLLESNSIADEELALINTQALLSGSTGEKQFISVSESTRTAPTSSEDYLRLKRRCTTSLIKEQESSQASTEECEKNKQDTITTKKYI

SEQ ID NO: 11 - protein, 3056 aa, Homo sapiens
MSLVLNDLLICCRQLEHDRATERKKEVEKFKRLIRDPETIKHLDRHSDSKQGKYLNWDAVFRFLQKYIQKETECLRIAKPNVSASTQASRQKKMQEISSLVKYFIKCANRRAPRLKCQELLNYIMDTVKDSSNGAIYGADCSNILLKDILSVRKYWCEISQQQWLELFSVYFRLYLKPSQDVHRVLVARIIHAVTKGCCSQTDGLNSKFLDFFSKAIQCARQEKSSSGLNHILAALTIFLKTLAVNFRIRVCELGDEILPTLLYIWTQHRLNDSLKEVIIELFQLQIYIHHPKGAKTQEKGAYESTKWRSILYNLYDLLVNEISHIGSRGKYSSGFRNIAVKENLIELMADICHQVFNEDTRSLEISQSYTTTQRESSDYSVPCKRKKIELGWEVIKDHLQKSQNDFDLVPWLQIATQLISKYPASLPNCELSPLLMILSQLLPQQRHGERTPYVLRCLTEVALCQDKRSNLESSQKSDLLKLWNKIWCITFRGISSEQIQAENFGLLGAIIQGSLVEVDREFWKLFTGSACRPSCPAVCCLTLALTTSIVPGTVKMGIEQNMCEVNRSFSLKESIMKWLLFYQLEGDLENSTEVPPILHSNFPHLVLEKILVSLTMKNCKAAMNFFQSVPECEHHQKDKEELSFSEVEELFLQTTFDKMDFLTIVRECGIEKHQSSIGFSVHQNLKESLDRCLLGLSEQLLNNYSSEITNSETLVRCSRLLVGVLGCYCYMGVIAEEEAYKSELFQKAKSLMQCAGESITLFKNKTNEEFRIGSLRNMMQLCTRCLSNCTKKSPNKIASGFFLRLLTSKLMNDIADICKSLASFIKKPFDRGEVESMEDDTNGNLMEVEDQSSMNLFNDYPDSSVSDANEPGESQSTIGAINPLAEEYLSKQDLLFLDMLKFLCLCVTTAQTNTVSFRAADIRRKLLMLIDSSTLEPTKSLHLHMYLMLLKELPGEEYPLPMEDVLELLKPLSNVCSLYRRDQDVCKTILNHVLHVVKNLGQSNMDSENTRDAQGQFLTVIGAFWHLTKERKYIFSVRMALVNCLKTLLEADPYSKWAILNVMGKDFPVNEVFTQFLADNHHQVRMLAAESINRLFQDTKGDSSRLLKALPLKLQQTAFENAYLKAQEGMREMSHSAENPETLDEIYNRKSVLLTLIAVVLSCSPICEKQALFALCKSVKENGLEPHLVKKVLEKVSETFGYRRLEDFMASHLDYLVLEWLNLQDTEYNLSSFPFILLNYTNIEDFYRSCYKVLIPHLVIRSHFDEVKSIANQIQEDWKSLLTDCFPKILVNILPYFAYEGTRDSGMAQQRETATKVYDMLKSENLLGKQIDHLFISNLPEIVVELLMTLHEPANSSASQSTDLCDFSGDLDPAPNPPHFPSHVIKATFAYISNCHKTKLKSILEILSKSPDSYQKILLAICEQAAETNNVYKKHRILKIYHLFVSLLLKDIKSGLGGAWAFVLRDVIYTLIHYINQRPSCIMDVSLRSFSLCCDLLSQVCQTAVTYCKDALENHLHVIVGTLIPLVYEQVEVQKQVLDLLKYLVIDNKDNENLYITIKLLDPFPDHVVFKDLRITQQKIKYSRGPFSLLEEINHFLSVSVYDALPLTRLEGLKDLRRQLELHKDQMVDIMRASQDNPQDGIMVKLVVNLLQLSKMAINHTGEKEVLEAVGSCLGEVGPIDFSTIAIQHSKDASYTKALKLFEDKELQWTFIMLTYLNNTLVEDCVKVRSAAVTCLKNILATKTGHSFWEIYKMTTDPMLAYLQPFRTSRKKFLEVPRFDKENPFEGLDDINLWIPLSENHDIWIKTLTCAFLDSGGTKCEILQLLKPMCEVKTDFCQTVLPYLIHDILLQDTNESWRNLLSTHVQGFFTSCLRHFSQTSRSTTPANLDSESEHFFRCCLDKKSQRTMLAVVDYMRRQKRPSSGTIFNDAFWLDLNYLEVAKVAQSCAAHFTALLYAEIYADKKSMDDQEKRSLAFEEGSQSTTISSLSEKSKEETGISLQDLLLEIYRSIGEPDSLYGCGGGKMLQPITRLRTYEHEAMWGKALVTYDLETAIPSSTRQAGIIQALQNLGLCHILSVYLKGLDYENKDWCPELEELHYQAAWRNMQWDHCTSVSKEVEGTSYHESLYNALQSLRDREFSTFYESLKYARVKEVEEMCKRSLESVYSLYPTLSRLQAIGELESIGELFSRSVTHRQLSEVYIKWQKHSQLLKDSDFSFQEPIMALRTVILEILMEKEMDNSQRECIKDILTKHLVELSILARTFKNTQLPERAIFQIKQYNSVSCGVSEWQLEEAQVFWAKKEQSLALSILKQMIKKLDASCAANNPSLKLTYTECLRVCGNWLAETCLENPAVIMQTYLEKAVEVAGNYDGESSDELRNGKMKAFLSLARFSDTQYQRIENYMKSSEFENKQALLKRAKEEVGLLREHKIQTNRYTVKVQRELELDELALRALKEDRKRFLCKAVENYINCLLSGEEHDMWVFRLCSLWLENSGVSEVNGMMKRDGMKIPTYKFLPLMYQLAARMGTKMMGGLGFHEVLNNLISRISMDHPHHTLFIILALANANRDEFLTKPEVARRSRITKNVPKQSSQLDEDRTEAANRIICTIRSRRPQMVRSVEALCDAYIILANLDATQWKTQRKGINIPADQPITKLKNLEDVVVPTMEIKVDHTGEYGNLVTIQSFKAEFRLAGGVNLPKIIDCVGSDGKERRQLVKGRDDLRQDAVMQQVFQMCNTLLQRNTETRKRKLTICTYKVVPLSQRSGVLEWCTGTVPIGEFLVNNEDGAHKRYRPNDFSAFQCQKKMMEVQKKSFEEKYEVFMDVCQNFQPVFRYFCMEKFLDPAIWFEKRLAYTRSVATSSIVGYILGLGDRHVQNILINEQSAELVHIDLGVAFEQGKILPTPETVPFRLTRDIVDGMGITGVEGVFRRCCEKTMEVMRNSQETLLTIVEVLLYDPLFDWTMNPLKALYLQQRPEDETELHPTLNADDQECKRNLSDIDQSFNKVAERVLMRLQEKLKGVEEGTVLSVGGQVNLLIQQAIDPKNLSRLFPGWKAWV

SEQ ID NO: 12 - protein, 777 aa, Homo sapiens
MPDNRQPRNRQPRIRSGNEPRSAPAMEPDGRGAWAHSRAALDRLEKLLRCSRCTNILREPVCLGGCEHIFCSNCVSDCIGTGCPVCYTPAWIQDLKINRQLDSMIQLCSKLRNLLHDNELSDLKEDKPRKSLFNDAGNKKNSIKMWFSPRSKKVRYVVSKASVQTQPAIKKDASAQQDSYEFVSPSPPADVSERAKKASARSGKKQKKKTLAEINQKWNLEAEKEDGEFDSKEESKQKLVSFCSQPSVISSPQINGEIDLLASGSLTESECFGSLTEVSLPLAEQIESPDTKSRNEVVTPEKVCKNYLTSKKSLPLENNGKRGHHNRLSSPISKRCRTSILSTSGDFVKQTVPSENIPLPECSSPPSCKRKVGGTSGRKNSNMSDEFISLSPGTPPSTLSSSSYRRVMSSPSAMKLLPNMAVKRNHRGETLLHIASIKGDIPSVEYLLQNGSDPNVKDHAGWTPLHEACNHGHLKVVELLLQHKALVNTTGYQNDSPLHDAAKNGHVDIVKLLLSYGASRNAVNIFGLRPVDYTDDESMKSLLLLPEKNESSSASHCSVMNTGQRRDGPLVLIGSGLSSEQQKMLSELAVILKAKKYTEFDSTVTHVVVPGDAVQSTLKCMLGILNGCWILKFEWVKACLRRKVCEQEEKYEIPEGPRRSRLNREQLLPKLFDGCYFYLWGTFKHHPKDNLIKLVTAGGGQILSRKPKPDSDVTQTINTVAYHARPDSDQRFCTQYIIYEDLCNYHPERVRQGKVWKAPSSWFIDCVMSFELLPLDS

SEQ ID NO: 13 - protein, 1249 aa, Homo sapiens
MSSMWSEYTIGGVKIYFPYKAYPSQLAMMNSILRGLNSKQHCLLESPTGSGKSLALLCSALAWQQSLSGKPADEGVSEKAEVQLSCCCACHSKDFTNNDMNQGTSRHFNYPSTPPSERNGTSSTCQDSPEKTTLAAKLSAKKQASIYRDENDDFQVEKKRIRPLETTQQIRKRHCFGTEVHNLDAKVDSGKTVKLNSPLEKINSFSPQKPPGHCSRCCCSTKQGNSQESSNTIKKDHTGKSKIPKIYFGTRTHKQIAQITRELRRTAYSGVPMTILSSRDHTCVHPEVVGNFNRNEKCMELLDGKNGKSCYFYHGVHKISDQHTLQTFQGMCKAWDIEELVSLGKKLKACPYYTARELIQDADIIFCPYNYLLDAQIRESMDLNLKEQVVILDEAHNIEDCARESASYSVTEVQLRFARDELDSMVNNNIRKKDHEPLRAVCCSLINWLEANAEYLVERDYESACKIWSGNEMLLTLHKMGITTATFPILQGHFSAVLQKEEKISPIYGKEEAREVPVISASTQIMLKGLFMVLDYLFRQNSRFADDYKIAIQQTYSWTNQIDISDKNGLLVLPKNKKRSRQKTAVHVLNFWCLNPAVAFSDINGKVQTIVLTSGTLSPMKSFSSELGVTFTIQLEANHIIKNSQVWVGTIGSGPKGRNLCATFQNTETFEFQDEVGALLLSVCQTVSQGILCFLPSYKLLEKLKERWLSTGLWHNLELVKTVIVEPQGGEKTNFDELLQVYYDAIKYKGEKDGALLVAVCRGKVSEGLDFSDDNARAVITIGIPFPNVKDLQVELKRQYNDHHSKLRGLLPGRQWYEIQAYRALNQALGRCIRHRNDWGALILVDDRFRNNPSRYISGLSKWVRQQIQHHSTFESALESLAEFSKKHQKVLNVSIKDRTNIQDNESTLEVTSLKYSTSPYLLEAASHLSPENFVEDEAKICVQELQCPKIITKNSPLPSSIISRKEKNDPVFLEEAGKAEKIVISRSTSPTFNKQTKRVSWSSFNSLGQYFTGKIPKATPELGSSENSASSPPRFKTEKMESKTVLPFTDKCESSNLTVNTSFGSCPQSETIISSLKIDATLTRKNHSEHPLCSEEALDPDIELSLVSEEDKQSTSNRDFETEAEDESIYFTPELYDPEDTDEEKNDLAETDRGNRLANNSDCILAKDLFEIRTIKEVDSAREVKAEDCIDTKLNGILHIEESKIDDIDGNVKTTWINELELGKTHEIEIKNFKPSPSKNKGMFPGFK

SEQ ID NO: 14 - protein, 882 aa, Homo sapiens
MGPWSRSLSALLLLLQVSSWLCQEPEPCHPGFDAESYTFTVPRRHLERGRVLGRVNFEDCTGRQRTAYFSLDTRFKVGTDGVITVKRPLRFHNPQIHFLVYAWDSTYRKFSTKVTLNTVGHHHRPPPHQASVSGIQAELLTFPNSSPGLRRQKRDWVIPPISCPENEKGPFPKNLVQIKSNKDKEGKVFYSITGQGADTPPVGVFIIERETGWLKVTEPLDRERIATYTLFSHAVSSNGNAVEDPMEILITVTDQNDNKPEFTQEVFKGSVMEGALPGTSVMEVTATDADDDVNTYNAAIAYTILSQDPELPDKNMFTINRNTGVISVVTTGLDRESFPTYTLVVQAADLQGEGLSTTATAVITVTDTNDNPPIFNPTTYKGQVPENEANVVITTLKVTDADAPNTPAWEAVYTILNDDGGQFVVTTNPVNNDGILKTAKGLDFEAKQQYILHVAVTNVVPFEVSLTTSTATVTVDVLDVNEAPIFVPPEKRVEVSEDFGVGQEITSYTAQEPDTFMEQKITYRIWRDTANWLEINPDTGAISTRAELDREDFEHVKNSTYTALIIATDNGSPVATGTGTLLLILSDVNDNAPIPEPRTIFFCERNPKPQVINIIDADLPPNTSPFTAELTHGASANWTIQYNDPTQESIILKPKMALEVGDYKINLKLMDNQNKDQVTTLEVSVCDCEGAAGVCRKAQPVEAGLQIPAILGILGGILALLILILLLLLFLRRRAVVKEPLLPPEDDTRDNVYYYDEEGGGEEDQDFDLSQLHRGLDARPEVTRNDVAPTLMSVPRYLPRPANPDEIGNFIDENLKAADTDPTAPPYDSLLVFDYEGSGSEAASLSSLNSSESDKDQDYDYLNEWGNRFKKLADMYGGGEDD

SEQ ID NO: 15 - protein, 543 aa, Homo sapiens
MSRESDVEAQQSHGSSACSQPHGSVTQSQGSSSQSQGISSSSTSTMPNSSQSSHSSSGTLSSLETVSTQELYSIPEDQEPEDQEPEEPTPAPWARLWALQDGFANLECVNDNYWFGRDKSCEYCFDEPLLKRTDKYRTYSKKHFRIFREVGPKNSYIAYIEDHSGNGTFVNTELVGKGKRRPLNNNSEIALSLSRNKVFVFFDLTVDDQSVYPKALRDEYIMSKTLGSGACGEVKLAFERKTCKKVAIKIISKRKFAIGSAREADPALNVETEIEILKKLNHPCIIKIKNFFDAEDYYIVLELMEGGELFDKVVGNKRLKEATCKLYFYQMLLAVQYLHENGIIHRDLKPENVLLSSQEEDCLIKITDFGHSKILGETSLMRTLCGTPTYLAPEVLVSVGTAGYNRAVDCWSLGVILFICLSGYPPFSEHRTQVSLKDQITSGKYNFIPEVWAEVSEKALDLVKKLLVVDPKARFTTEEALRHPWLQDEDMKRKFQDLLSEENESTALPQVLAQPSTSRKRPREGEAEGAETTKRPAVCAAVL

SEQ ID NO: 16 - protein, 314 aa, Homo sapiens
MAPPQVLAFGLLLAAATATFAAAQEECVCENYKLAVNCFVNNNRQCQCTSVGAQNTVICSKLAAKCLVMKAEMNGSKLGRRAKPEGALQNNDGLYDPDCDESGLFKAKQCNGTSMCWCVNTAGVRRTDKDTEITCSERVRTYWIIIELKHKAREKPYDSKSLRTALQKEITTRYQLDPKFITSILYENNVITIDLVQNSSQKTQNDVDIADVAYYFEKDVKGESLFHSKKMDLTVNGEQLDLDPGQTLIYYVDEKAPEFSMQGLKAGVIAVIVVVVIAVVAGIVVLVISRKKRMAKYEKAEIKEMGEMHRELNA

SEQ ID NO: 17 - protein, 756 aa, Homo sapiens
MSFVAGVIRRLDETVVNRIAAGEVIQRPANAIKEMIENCLDAKSTSIQVIVKEGGLKLIQIQDNGTGIRKEDLDIVCERFTTSKLQSFEDLASISTYGFRGEALASISHVAHVTITTKTADGKCAYRASYSDGKLKAPPKPCAGNQGTQITVEDLFYNIATRRKALKNPSEEYGKILEVVGRYSVHNAGISFSVKKQGETVADVRTLPNASTVDNIRSIFGNAVSRELIEIGCEDKTLAFKMNGYISNANYSVKKCIFLLFINHRLVESTSLRKAIETVYAAYLPKNTHPFLYLSLEISPQNVDVNVHPTKHEVHFLHEESILERVQQHIESKLLGSNSSRMYFTQTLLPGLAGPSGEMVKSTTSLTSSSTSGSSDKVYAHQMVRTDSREQKLDAFLQPLSKPLSSQPQAIVTEDKTDISSGRARQQDEEMLELPAPAEVAAKNQSLEGDTTKGTSEMSEKRGPTSSNPRKRHREDSDVEMVEDDSRKEMTAACTPRRRIINLTSVLSLQEEINEQGHEVLREMLHNHSFVGCVNPQWALAQHQTKLYLLNTTKLSEELFYQILIYDFANFGVLRLSEPAPLFDLAMLALDSPESGWTEEDGPKEGLAEYIVEFLKKKAEMLADYFSLEIDEEGNLIGLPLLIDNYVPPLEGLPIFILRLATEVNWDEEKECFESLSKECAMFYSIRKQYISEESTLSGQQSEVPGSIPNSWKWTVEHIVYKALRSHILPPKHFTEDGNILQLANLPDLYKVFERC

SEQ ID NO: 18 - protein, 934 aa, Homo sapiens [sequence omitted]

SEQ ID NO: 19 - protein, 1360 aa, Homo sapiens
MSRQSTLYSFFPKSPALSDANKASARASREGGRAAAAPGASPSPGGDAAWSEAGPGPRPLARSASPPKAKNLNGGLRRSVAPAAPTSCDFSPGDLVWAKMEGYPWWPCLVYNHPFDGTFIREKGKSVRVHVQFFDDSPTRGWVSKRLLKPYTGSKSKEAQKGGHFYSAKPEILRAMQRADEALNKDKIKRLELAVCDEPSEPEEEEEMEVGTTYVTDKSEEDNEIESEEEVQPKTQGSRRSSRQIKKRRVISDSESDIGGSDVEFKPDTKEEGSSDEISSGVGDSESEGLNSPVKVARKRKRMVTGNGSLKRKSSRKETPSATKQATSISSETKNTLRAFSAPQNSESQAHVSGGGDDSSRPTVWYHETLEWLKEEKRRDEHRRRPDHPDFDASTLYVPEDFLNSCTPGMRKWWQIKSQNFDLVICYKVGKFYELYHMDALIGVSELGLVFMKGNWAHSGFPEIAFGRYSDSLVQKGYKVARVEQTETPEMMEARCRKMAHISKYDRVVRREICRIITKGTQTYSVLEGDPSENYSKYLLSLKEKEEDSSGHTRAYGVCFVDTSLGKFFIGQFSDDRHCSRFRTLVAHYPPVQVLFEKGNLSKETKTILKSSLSCSLQEGLIPGSQFWDASKTLRTLLEEEYFREKLSDGIGVMLPQVLKGMTSESDSIGLTPGEKSELALSALGGCVFYLKKCLIDQELLSMANFEEYIPLDSDTVSTTRSGAIFTKAYQRMVLDAVTLNNLEIFLNGTNGSTEGTLLERVDTCHTPFGKRLLKQWLCAPLCNHYAINDRLDAIEDLMVVPDKISEVVELLKKLPDLERLLSKIHNVGSPLKSQNHPDSRAIMYEETTYSKKKIIDFLSALEGFKVMCKIIGIMEEVADGFKSKILKQVISLQTKNPEGRFPDLTVELNRWDTAFDHEKARKTGLITPKAGFDSDYDQALADIRENEQSLLEYLEKQRNRIGCRTIVYWGIGRNRYQLEIPENFTTRNLPEEYELKSTKKGCKRYWTKTIEKKLANLINAEERRDVSLKDCMRRLFYNFDKNYKDWQSAVECIAVLDVLLCLANYSRGGDGPMCRPVILLPEDTPPFLELKGSRHPCITKTFFGDDFIPNDILIGCEEEEQENGKAYCVLVTGPNMGGKSTLMRQAGLLAVMAQMGCYVPAEVCRLTPIDRVFTRLGASDRIMSGESTFFVELSETASILMHATAHSLVLVDELGRGTATFDGTAIANAVVKELAETIKCRTLFSTHYHSLVEDYSQNVAVRLGHMACMVENECEDPSQETITFLYKFIKGACPKSYGFNAARLANLPEEVIQKGHRKAREFEKMNQSLRLFREVCLASERSTVDAEAVHKLLTLIKEL

SEQ ID NO: 20 - protein, 754 aa, Homo sapiens
MWKLLPAAGPAGGEPYRLLTGVEYVVGRKNCAILIENDQSISRNHAVLTANFSVTNLSQTDEIPVLTLKDNSKYGTFVNEEKMQNGFSRTLKSGDGITFGVFGSKFRIEYEPLVACSSCLDVSGKTALNQAILQLGGFTVNNWTEECTHLVMVSVKVTIKTICALICGRPIVKPEYFTEFLKAVESKKQPPQIESFYPPLDEPSIGSKNVDLSGRQERKQIFKGKTFIFLNAKQHKKLSSAVVFGGGEARLITEENEEEHNFFLAPGTCVVDTGITNSQTLIPDCQKKWIQSIMDMLQRQGLRPIPEAEIGLAVIFMTTKNYCDPQGHPSTGLKTTTPGPSLSQGVSVDEKLMPSAPVNTTTYVADTESEQADTWDLSERPKEIKVSKMEQKFRMLSQDAPTVKESCKTSSNNNSMVSNTLAKMRIPNYQLSPTKLPSINKSKDRASQQQQTNSIRNYFQPSTKKRERDEENQEMSSCKSARIETSCSLLEQTQPATPSLWKNKEQHLSENEPVDTNSDNNLFTDTDLKSIVKNSASKSHAAEKLRSNKKREMDDVAIEDEVLEQLFKDTKPELEIDVKVQKQEEDVNVRKRPRMDIETNDTFSDEAVPESSKISQENEIGKKRELKEDSLWSAKEISNNDKLQDDSEMLPKKLLLTEFRSLVIKNSTSRNPSGINDDYGQLKNFKKFKKVTYPGAGKLPHIIGGSDLIAHHARKNTELEEWLRQEMEVQNQHAKEESLADDLFRYNPYLKRRR

SEQ ID NO: 21 - protein, 1186 aa, Homo sapiens [sequence omitted]

SEQ ID NO: 22 - protein, 403 aa, Homo sapiens [sequence omitted]

SEQ ID NO: 23 - protein, 328 aa, Homo sapiens
MGVLRVGLCPGLTEEMIQLLRSHRIKTVVDLVSADLEEVAQKCGLSYKALVALRRVLLAQFSAFPVNGADLYEELKTSTAILSTGIGSLDKLLDAGLYTGEVTEIVGGPGSGKTQVCLCMAANVAHGLQQNVLYVDSNGGLTASRLLQLLQAKTQDEEEQAEALRRIQVVHAFDIFQMLDVLQELRGTVAQQVTGSSGTVKVVVVDSVTAVVSPLLGGQQREGLALMMQLARELKTLARDLGMAVVVTNHITRDRDSGRLKPALGRSWSFVPSTRILLDTIEGAGASGGRRMACLAKSSRQPTGFQEMVDIGTWGTSEQSATLQGDQT

SEQ ID NO: 24 - protein, 433 aa, Homo sapiens
MEVVDPQQLGMFTEGELMSVGMDTFIHRIDSTEVIYQPRRKRAKLIGKYLMGDLLGEGSYGKVKEVLDSETLCRRAVKILKKKKLRRIPNGEANVKKEIQLLRRLRHKNVIQLVDVLYNEEKQKMYMVMEYCVCGMQEMLDSVPEKRFPVCQAHGYFCQLIDGLEYLHSQGIVHKDIKPGNLLLTTGGTLKISDLGVAEALHPFAADDTCRTSQGSPAFQPPEIANGLDTFSGFKVDIWSAGVTLYNITTGLYPFEGDNIYKLFENIGKGSYAIPGDCGPPLSDLLKGMLEYEPAKRFSIRQIRQHSWFRKKHPPAEAPVPIPPSPDTKDRWRSMTVVPYLEDLHGADEDEDLFDIEDDIIYTQDFTVPGQVPEEEASHNGQRRGLPKAVCMNGTEAAQLSTKSRAEGRAPNPARKACSASSKIRRLSACKQQ

SEQ ID NO: 25 - protein, 393 aa, Homo sapiens [sequence omitted]

SEQ ID NO: 26 - protein, 189 aa, Homo sapiens [sequence omitted]

(19) United States
(12) Patent Application Publication
(10) Pub. No.: US 2023/0127069 A1
(43) Pub. Date: Apr. 27, 2023

(54) COMPOSITIONS FOR OVARIAN CANCER ASSESSMENT HAVING IMPROVED SPECIFICITY AND SENSITIVITY

(71) Applicant: Aspira Women's Health Inc., Austin, TX (US)

(72) Inventors: Herbert FRITSCHE, Austin, TX (US); Lesley NORTHROP, Austin, TX (US)

(73) Assignee: Aspira Women's Health Inc., Austin, TX (US)

(21) Appl. No.: 17/933,446

(22) Filed: Sep. 19, 2022

Related U.S. Application Data
(63) Continuation of application No. PCT/US2021/023091, filed on Mar. 19, 2021.
(60) Provisional application No. 62/992,358, filed on Mar. 20, 2020.

(51) Int. Cl. G01N 33/574

(52) U.S. Cl. CPC G01N 33/57449; G01N 2333/775; G01N 2333/79; G01N 2333/59

(57) ABSTRACT

The present invention provides compositions and methods having improved specificity and sensitivity for the pre-operative assessment of ovarian tumors (e.g., symptomatic and asymptomatic adnexal mass) in a variety of subjects (e.g., pre- and post-menopausal women) having a variety of ovarian cancer types (e.g., low malignant potential, intermediate malignant potential, high malignant potential).

### CROSS REFERENCE TO RELATED APPLICATIONS

This application is a continuation application, pursuant to 35 U.S.C. § 111(a) of PCT International Application No. PCT/US2021/023091, filed Mar. 19, 2021, designating the United States and published in English, which claims the benefit of and priority to U.S. Provisional Application No. 62/992,358, filed Mar. 20, 2020, the entire contents of each of which are incorporated herein by reference in their entirety.

### SEQUENCE LISTING

The present application contains a Sequence Listing which has been submitted electronically in XML format. The content of the electronic XML Sequence Listing, (Date of creation: Sep. 18, 2022; Size: 219,414 bytes; Name: 168109-012802US-SequenceListing.xml) is herein incorporated by reference in its entirety.

### BACKGROUND OF THE INVENTION

Ovarian cancer is among the most lethal gynecologic malignancies in developed countries. Annually in the United States alone, approximately 23,000 women are diagnosed with the disease and almost 14,000 women die from it. Despite progress in cancer therapy, ovarian cancer mortality has remained virtually unchanged over the past two decades. Given the steep survival gradient relative to the stage at which the disease is diagnosed, early detection remains the most important factor in improving long-term survival of ovarian cancer patients. A second important factor is whether or not women with ovarian cancer are treated by a surgeon that specializes in gynecological oncology.

The importance of identifying women who should be treated by a gynecological oncologist is highlighted in a consensus statement issued by the National Institutes of Health (NIH). In 1994, the NIH indicated that women identified preoperatively as having a significant risk of ovarian cancer should have the option of having their surgery performed by a gynecologic oncologist. To ensure that no woman who has ovarian cancer is overlooked, current diagnostic methods optimize sensitivity at the expense of specificity. Present diagnostic methods have an unacceptably high false positive rate. In human terms, this means that fifty percent of women go into surgery believing that they have ovarian cancer when in fact they have a benign mass. There is an urgent need for improved diagnostic methods that not only have a high degree of sensitivity, but that also provide a high degree of specificity, which can be used to manage subject treatment more effectively and ensure that the appropriate patients are being promptly and properly referred to specialists.

### SUMMARY OF THE INVENTION

The present invention provides compositions and methods having improved specificity and sensitivity for the pre-operative assessment of ovarian tumors (e.g., symptomatic and asymptomatic adnexal mass) in a variety of subjects (e.g., pre- and post-menopausal women) having a variety of ovarian cancer types (e.g., low malignant potential, intermediate malignant potential, high malignant potential).

In one aspect, the invention provides a panel for pre-operatively assessing a subject's risk of having ovarian cancer, the panel comprising and or consisting of markers Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH), and one or more markers selected from the group consisting of Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P161NK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1).

In one aspect, the invention provides a panel for pre-operatively assessing a subject's risk of having ovarian cancer, the panel comprising or consisting of markers Transthyretin/prealbumin (TT), Apolipoprotein A1 (ApoA1), β2-Microglobulin (β2M), Transferrin (Tfr), Cancer Antigen 125 (CA125), and one or more markers selected from the group consisting of Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), 06-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P16INK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1).

In one aspect, the invention provides a panel for pre-operatively assessing a subject's risk of having ovarian cancer, the panel comprising or consisting of markers Apolipoprotein A1 (ApoA1), Transferrin (Tfr), Cancer Antigen 125 (CA125), Human epididymis protein 4 (HE4), follicle stimulating hormone (FSH), and one or more markers selected from the group consisting of Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P161NK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1).

In one aspect, the invention provides a panel for pre-operatively assessing a subject's risk of having ovarian cancer, the panel comprising or consisting of markers Transthyretin/prealbumin (TT), Apolipoprotein A1 (ApoA1), β2-Microglobulin (β2M), Transferrin (Tfr), Cancer Antigen 125 (CA125), and Breast Cancer 1 (BRCA1).

In one aspect, the invention provides a panel for pre-operatively assessing a subject's risk of having ovarian cancer, the panel comprising or consisting of markers Transthyretin/prealbumin (TT), Apolipoprotein A1 (ApoA1), β2-Microglobulin (β2M), Transferrin (Tfr), Cancer Antigen 125 (CA125), and Breast Cancer 2 (BRCA2).

In one aspect, the invention provides a panel for pre-operatively assessing a subject's risk of having ovarian cancer, the panel comprising or consisting of markers Transthyretin/prealbumin (TT), Apolipoprotein A1 (ApoA1), β2-Microglobulin (β2M), Transferrin (Tfr), Cancer Antigen 125 (CA125), Breast Cancer 1 (BRCA1) and Breast Cancer 2 (BRCA2).

In one aspect, the invention provides a panel for pre-operatively assessing a subject's risk of having ovarian cancer, the panel comprising or consisting of markers Apolipoprotein A1 (ApoA1), Transferrin (Tfr), Cancer Antigen 125 (CA125), HE4, follicle stimulating hormone (FSH), and Breast Cancer 1 (BRCA1).

In one aspect, the invention provides a panel for pre-operatively assessing a subject's risk of having ovarian cancer, the panel comprising or consisting of markers Apolipoprotein A1 (ApoA1), Transferrin (Tfr), Cancer Antigen 125 (CA125), HE4, follicle stimulating hormone (FSH), and Breast Cancer 2 (BRCA2).

In one aspect, the invention provides a panel for pre-operatively assessing a subject's risk of having ovarian cancer, the panel comprising or consisting of markers Apolipoprotein A1 (ApoA1), Transferrin (Tfr), Cancer Antigen 125 (CA125), Human epididymis protein 4 (HE4), follicle stimulating hormone (FSH), Breast Cancer 1 (BRCA1) and Breast Cancer 2 (BRCA2).

In one aspect, the invention provides a panel for pre-operatively assessing a subject's risk of having ovarian cancer, the panel comprising or consisting of markers Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), follicle stimulating hormone (FSH), and Breast Cancer 1 (BRCA1).

In one aspect, the invention provides a panel for pre-operatively assessing a subject's risk of having ovarian cancer, the panel comprising or consisting of markers Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), follicle stimulating hormone (FSH), and Breast Cancer 2 (BRCA2).

In one aspect, the invention provides a panel for pre-operatively assessing a subject's risk of having ovarian cancer, the panel comprising or consisting of markers Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), follicle stimulating hormone (FSH), Breast Cancer 1 (BRCA1), and Breast Cancer 2 (BRCA2).

In some embodiments, the panels of the invention further include one or more markers selected from the group consisting of Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P161NK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1).

In some embodiments, each of the markers are bound to a separate capture reagent. In one embodiment, the capture reagents are attached to a solid support. In one embodiment, the solid support is a plate, chip, beads, microfluidic platform, membrane, planar microarray, or suspension array. In one embodiment, the capture reagent is an antibody, aptamer, Affibody, hybridization probe and/or fragments thereof. In one embodiment, each capture reagent specifically binds to one of the markers.

In some aspects, an of the panels of the invention may be used in a method for pre-operatively assessing a subject's risk of having ovarian cancer.

In one aspect, the invention provides a method for pre-operatively assessing a subject's risk of having ovarian cancer, the method comprising characterizing markers in a biological sample from the subject using any of the panels as provided herein.

In one aspect, the invention provides a method for pre-operatively assessing a subject as having a high or a low risk of ovarian cancer, the method comprising, (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a biological sample derived from the subject to determine a first score, wherein the first score identifies the subject as having a high, intermediate or low cancer risk; and (b) characterizing markers comprising or consisting of CA125, β2M, Tfr, TT and ApoA1 in the biological sample derived from the subject identified by the first score as having an intermediate cancer risk to determine a second score, wherein the second score identifies the subject as having a low or high cancer risk.

In one aspect, the invention provides a method for pre-operatively assessing a subject as having a high or low risk of ovarian cancer, the method comprising, (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a biological sample derived from the subject to determine a first score, wherein the first score identifies the subject as having a high, intermediate or low cancer risk; and (b) characterizing markers comprising or consisting of FSH, CA125, HE4, Tfr, and ApoA1 in the biological sample derived from the subject identified by the first score as having an intermediate cancer risk to determine a second score, wherein the second score identifies the subject as having a low or high cancer risk.

In one aspect, the invention provides a method for pre-operatively assessing a subject as having a high or low risk of ovarian cancer, the method comprising, (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a biological sample derived from the subject to determine first score, wherein the first score identifies the subject as having a high, intermediate or low cancer risk; (b) characterizing markers comprising or consisting of CA125, β2M, Tfr, TT and ApoA1 in the biological sample derived from the subject identified by the first score as having an intermediate cancer risk to determine a second score, which identifies the subject as low, intermediate, or high risk; and (c) characterizing markers comprising or consisting of FSH, CA125, HE4, Tfr, and ApoA1 in the biological sample derived from the subject identified by the second score as having an intermediate or high cancer risk to determine a third score, wherein the third score identifies the subject as having a low or high cancer risk.

In one aspect, the invention provides a method for pre-operatively assessing an asymptomatic subject, the method comprising, (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a biological sample derived from the subject to determine first score, wherein the first score identifies the subject as having a high, intermediate or low cancer risk; and (b) characterizing one or more markers selected from the group consisting of Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDHT), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAMT75A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type TA (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P161NK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1) in the biological sample derived from the subject identified by the first score as having a high, intermediate or low cancer risk, wherein the presence of one or more mutations in one or more markers or the presence of an aberrant methylation in one or more markers identifies the subject as having a higher [increased] cancer risk relative to a subject that does not have a mutation or an aberrant methylation in the one or more markers. In one embodiment, the presence of one or more mutations in one or more markers or the presence of an aberrant methylation in one or more markers identifies the subject as in need of therapeutic intervention. In one embodiment, the presence of one or more mutations in one or more markers or the presence of an aberrant methylation in one or more markers identifies the subject identified as having a high cancer risk as in need of therapeutic intervention. In one embodiment, the aberrant methylation is hypermethylation. In one embodiment, the aberrant methylation is hypomethylation. In one embodiment, the therapeutic intervention is surgery.

In some embodiments, the methods of the invention further include characterizing one or more markers selected from the group consisting of Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), and Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXADI1), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P16INK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1) in the biological sample derived from the subject identified by the first score as having a high, intermediate or low cancer risk, wherein the presence of one or more mutations in one or more markers or the presence of an aberrant methylation in one or more markers identifies the subject as having a higher [increased] cancer risk relative to a subject that does not have one or more mutations or an aberrant methylation in the one or more markers. In one embodiment, the presence of one or more mutations in one or more markers or the presence of an aberrant methylation in one or more markers identifies the subject as in need of therapeutic intervention. In one embodiment, the presence of one or more mutations in one or more markers or the presence of an aberrant methylation in one or more markers identifies the subject identified as having a high cancer risk as in need of therapeutic intervention. In one embodiment, the aberrant methylation is hypermethylation. In one embodiment, the aberrant methylation is hypomethylation. In one embodiment, the therapeutic intervention is surgery.

In one aspect, the invention provides a method for pre-operatively monitoring a subject with one or more mutations or an aberrant methylation in one or more germline and/or somatic markers, the method comprising: (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a first biological sample derived from the subject to determine a first score at a first time point, wherein the first score identifies the subject as having a high, intermediate or low ovarian cancer risk; and (b) repeating step (a) in one or more biological samples from the subject identified as having an intermediate or low ovarian cancer risk at one or more time points, thereby monitoring the subject.

In one aspect, the invention provides a method for pre-operatively monitoring a subject with one or more mutations or an aberrant methylation in one or more germline and/or somatic markers, the method comprising: (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a first biological sample derived from the subject to determine a first score at a first time point, wherein the first score identifies the subject as having a high, intermediate or low ovarian cancer risk; (b) characterizing markers comprising or consisting of CA125, β2M, Tfr, TT and ApoA1 in the biological sample derived from the subject identified by the first score as having a low or intermediate cancer risk to determine a second score at the first time point, wherein the second score identifies the subject as having a low or high cancer risk; and (c) repeating steps (a) and (b) in one or more biological samples from the subject identified as having low ovarian cancer risk in step (b) at one or more time points, thereby monitoring the subject.

In one aspect, the invention provides a method for pre-operatively monitoring a subject with one or more mutations or an aberrant methylation in one or more germline and/or somatic markers, the method comprising: (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a first biological sample derived from the subject to determine a first score at a first time point, wherein the first score identifies the subject as having a high, intermediate or low ovarian cancer risk; (b) characterizing markers comprising or consisting of FSH, CA125, HE4, Tfr, and ApoA1 in the biological sample derived from the subject identified by the first score as having a low or intermediate cancer risk to determine a second score at the first time point, wherein the second score identifies the subject as having a low or high cancer risk; and (c) repeating steps (a) and (b) in one or more biological samples from the subject identified as having low ovarian cancer risk in step (b) at one or more time points, thereby monitoring the subject.

In one aspect, the invention provides a method for pre-operatively monitoring a subject with one or more mutations or an aberrant methylation in one or more germline and/or somatic markers identified as having a low or intermediate ovarian cancer risk, the method comprising: (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a first biological sample derived from the subject to determine a first score at a first time point, wherein the first score identifies the subject as having a high, intermediate or low ovarian cancer risk; (b) characterizing markers comprising or consisting of CA125, β2M, Tfr, TT and ApoA1 in the biological sample derived from the subject identified by the first score as having a low, intermediate cancer risk to determine a second score at the first time point, wherein the second score identifies the subject as having a low, intermediate or high cancer risk; (c) characterizing markers comprising or consisting of FSH, CA125, HE4, Tfr, and ApoA1 in the biological sample derived from the subject identified by the second score as having an intermediate or high cancer risk to determine a third score at the first time point, wherein the third score identifies the subject as having a low or high cancer risk; and (d) repeating steps (a)-(c) in one or more biological samples from the subject identified as having a low or intermediate risk in step (b) or a low ovarian cancer risk in step (c) at one or more time points, thereby monitoring the subject.

In some embodiments, the one or more germline and/or somatic markers are selected from the group consisting of Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), and Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P16INK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1).

In one aspect, the invention provides a method for pre-operatively monitoring a subject with an adnexal mass, the method comprising, (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a biological sample derived from the subject to determine a first score at a first time point, wherein the first score identifies the subject as having a high, intermediate or low ovarian cancer risk; (b) characterizing markers comprising or consisting of CA125, β2M, Transferrin, Transthyretin and ApoA1 in the biological sample derived from the subject identified by the first score as having an intermediate cancer risk to determine a second score at the first time point, wherein the second score identifies the subject as having a low or high cancer risk; and (c) repeating steps (a) and (b) in one or more biological samples from the subject identified as having low risk of ovarian cancer risk in step (b) at one or more time points, thereby monitoring the subject.

In one aspect, the invention provides a method for pre-operatively monitoring a subject with an adnexal mass, the method comprising, (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a biological sample derived from the subject to determine a first score at a first time point, wherein the first score identifies the subject as having a high, intermediate or low ovarian cancer risk; (b) characterizing markers comprising or consisting of FSH, CA125, HE4, Tfr, and ApoA1 in the biological sample derived from the subject identified by the first score as having an intermediate cancer risk to determine a second score at the first time point, wherein the second score identifies the subject as having a low or high ovarian cancer risk; and (c) repeating steps (a) and (b) in one or more biological samples from the subject identified as having low risk of ovarian cancer risk in step (b) at one or more time points, thereby monitoring the subject.

In one aspect, the invention provides a method for monitoring a subject with an adnexal mass, the method comprising, (a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a first biological sample derived from the subject to determine a first score at a first time point, wherein the first score identifies the subject as having a high, intermediate or low risk of ovarian cancer; (b) characterizing markers comprising or consisting of CA125, β2M, Tfr, TT and ApoA1 in the biological sample derived from the subject identified by the first score as having a low or intermediate cancer risk to determine a second score at the first time point, wherein the second score identifies the subject as having a low, intermediate, or high ovarian cancer risk; (c) characterizing markers comprising or consisting of FSH, CA125, HE4, Tfr, and ApoA1 in the biological sample derived from the subject identified by the first score as having an intermediate or high cancer risk to determine a third score at the first time point, wherein the third score identifies the subject as having a low or high cancer risk; and (d) repeating steps (a)-(c) in one or more biological samples from the subject identified as having a low or intermediate risk in step (b) or a low risk of ovarian cancer risk in step (c) at one or more time points, thereby monitoring the subject.

In some embodiments, the one or more germline markers are BRCA1 and/or BRCA2. In one embodiment, the one or more mutations in BRCA1 comprises c. 68_69del and/or c.5266dup, and/or the one or more mutations in BRCA2 comprises c.5946del. In one embodiment, the one or more markers are characterized by detecting cell-free tumor DNA (cftDNA). In one embodiment, the markers are characterized by immunoassay, sequencing and/or nucleic acid microarray. In one embodiment, the sequencing is next-generation sequencing (NGS) or Sanger sequencing. In one embodiment, the immunoassay comprises affinity capture assay, immunometric assay, heterogeneous chemiluminscence immunometric assay, homogeneous chemiluminscence immunometric assay, ELISA, western blotting, radioimmunoassay, magnetic immunoassay, real-time immunoquantitative PCR (iqPCR) and SERS label free assay.

In some embodiments, the first score ranges from 0 to 20, and wherein a first score less than or equal to 5 identifies the subject as having a low cancer risk, a first score greater than 5 and less than 10 in a pre-menopausal subject or a first score greater than 5 and less than 14 in a post-menopausal subject identifies the subject as having an intermediate cancer risk, and a first score greater than or equal to 10 in a pre-menopausal subject or a first score greater than or equal to 14 in a post-menopausal subject identifies the subject as having a high cancer risk. In some embodiments, the second score ranges from 0 to 20, and wherein a second score less than 5 identifies the subject as having a low cancer risk and a first score of 5 or greater identifies the subject as having a high cancer risk. In some embodiments, the second score ranges from 0 to 20, and wherein a second score less than or equal to 5 in a pre-menopausal subject or a second score less than 4.4 in a post-menopausal subject identifies the subject as having a low cancer risk, a second score greater than 5 and less than 7 in a pre-menopausal subject or a second score greater than 4.4 and less than 6 in a post-menopausal subject identifies the subject as having an intermediate cancer risk, and a second score greater than or equal to 7 in a pre-menopausal subject or a second score greater than or equal to 6 in a post-menopausal subject identifies the subject as having a high cancer risk. In some embodiments, the third score ranges from 0 to 20, and wherein a third score less than or equal to 5 identifies the subject as having a low cancer risk and a third score greater than 5 identifies the subject as having a high cancer risk.

In some embodiments, each of the markers are bound to a separate capture reagent. In one embodiment, the capture reagents are attached to a solid support. In one embodiment, the solid support is a plate, chip, beads, microfluidic platform, membrane, planar microarray, or suspension array. In one embodiment, the capture reagent is an antibody, aptamer, Affibody, hybridization probe and/or fragments thereof. In one embodiment, each capture reagent specifically binds to one of the markers.

In some embodiments, the methods of the invention further characterize one or more clinical biomarkers of ovarian cancer risk in the subject, wherein the one or more clinical biomarkers are selected from group consisting of age, pre-menopausal status, post-menopausal status, ethnicity, pathology, adnexal mass diagnosis, family history, physical examination, imaging results, and/or history of smoking, wherein the one or more clinical biomarkers further identifies the subject as having a low or high cancer risk.

In one aspect, the invention provides a method for classifying a subject's risk of having ovarian cancer, the method comprising: receiving, by at least one processor, a first panel signal representing a marker spectrum peak detected for each marker of a panel comprising or consisting of markers Transthyretin/prealbumin (TT), Apolipoprotein A1 (ApoA1), β2-Microglobulin (β2M), Transferrin (Tfr), Cancer Antigen 125 (CA125), HE4, and follicle stimulating hormone (FSH) and one or more markers selected from the group consisting of Breast Cancer 1 (BRCA1), Breast Cancer 2 (BRCA2), ATM, BARD1, BRIP1, CDH1, CHEK2, EPCAM, MLH1, MSH2, MSH6, NBN, PALB2, PTEN, RAD51D, STK11, TP53, KRAS, ABRAXAS1, AKT1, APC, AXIN2, BMPR1A, BRAF, CDC25, CDKN2A, CDK4, CTNNB1, DICER1, ERBB2, ERCC6, FANCM, FANCC, MRE11, MUTYH, NF1, NTHL1, PIK3CA, PMS2, PP2R1A, PRKDC, POLD1, RAD50, RAD51C, RNF43, SDHB, SDHD, SMARCA4, XRCC2, WRN, CDC73, GALNT12, GREM1, HOXB13, MSH3, POLE, RAD51, RINT1, RSP20, SLX4, SMAD4, TTK, RASSFlA, RUNX3, TFPI2, SFRP5, OPCML, MGMT, CDH13, SULF1, HOXA9, HOXAD11, CLDN4, MAL, BORIS, ABCG2, TUBB3, MCJ, SNGG, P14ARF, P16INK4A, DAPK, P15, MINT31, RIZ1, and TMS1; utilizing, by the at least one processor, a first stage cancer risk classifier to predict a cancer risk classification score representative of a predicted risk of developing ovarian cancer, the cancer risk classification score being based on learned risk classification parameters and the first panel signal; determining, by the at least one processor, a cancer risk level associated with the cancer risk classification score, the cancer risk level selected from one of at least the selection comprising low risk, intermediate risk and high risk; and generating, by the at least one processor, a cancer risk level prediction at a computing device associated with a care provider indicative of the cancer risk level of the subject.

In some embodiments, the methods of the invention further include determining, by the at least one processor, the cancer risk level as intermediate risk; utilizing, by the at least one processor, a second stage cancer risk classifier to predict an enhanced cancer risk classification score based on second stage learned risk classification parameters and second panel signal comprising a subset of the first panel signal; and determining, by the at least one processor, an enhanced cancer risk level associated with the enhanced cancer risk classification score, the enhanced cancer risk level selected from one of at least the selection comprising low risk and high risk. In one embodiment, the second panel signal represents the marker spectrum peak of markers comprising or consisting of CA125, β2M, Transferrin, Transthyretin and ApoA1. In one embodiment, the second panel signal represents the marker spectrum peak of markers comprising or consisting of FSH, CA125, HE4, Transferrin, ApoA1.

In some embodiments, the methods of the invention further include determining, by the at least one processor, the cancer risk level as intermediate risk; utilizing, by the at least one processor, a second stage cancer risk classifier to predict an enhanced cancer risk classification score based on second stage learned risk classification parameters and second panel signals comprising a different subset of the first panel signal; utilizing, by the at least one processor, a third stage cancer risk classifier to predict an enhanced cancer risk classification score based on second stage learned risk classification parameters and third panel signals comprising a different subset of the first panel signal; and determining, by the at least one processor, an enhanced cancer risk level associated with the enhanced cancer risk classification score, the enhanced cancer risk level selected from one of at least the selection comprising low risk and high risk. In one embodiment, the second panel signal represents the marker spectrum peak of markers comprising or consisting of CA125, β2M, Transferrin, Transthyretin and ApoA1 and the third panel signal represents the marker spectrum peak of markers comprising or consisting of FSH, CA125, HE4, Transferrin, ApoA1.

In some embodiments, the methods of the invention further include determining, by the at least one processor, the low risk of the cancer risk level where the cancer risk classification score is between 0.0 and 5.0; determining, by the at least one processor, the intermediate risk of the cancer risk level where the cancer risk classification score is between 5.1 and 9.9; and determining, by the at least one processor, the high risk of the cancer risk level where the cancer risk classification score is between 10.0 and 20.0. In some embodiments, the first stage cancer risk classifier comprises: a pre-menopausal first stage cancer risk prediction model having learned pre-menopausal risk classification parameters of the learned risk classification parameters; and a post-menopausal first stage cancer risk prediction model having learned post-menopausal risk classification parameters of the learned risk classification parameters.

In some embodiments, the methods of the invention further include determining, by the at least one processor, the low risk of the cancer risk level where the cancer risk classification score is between 0.0 and 5.0; determining, by the at least one processor, the intermediate risk of the cancer risk level where the cancer risk classification score is between 5.1 and 13.9 for a post-menopausal subject; and determining, by the at least one processor, the low risk of the cancer risk level where the cancer risk classification score is between 14.0 and 20.0 for a post-menopausal subject.

In some embodiments, the methods of the invention further include determining, by the at least one processor, the low risk of the cancer risk level where the cancer risk classification score is between 0.0 and 5.0; determining, by the at least one processor, the intermediate risk of the cancer risk level where the cancer risk classification score is between 5.1 and 9.9 for a pre-menopausal subject; and determining, by the at least one processor, the high risk of the cancer risk level where the cancer risk classification score is between 10.0 and 20.0 for a pre-menopausal subject.

In some embodiments, the methods of the invention further include generating, by the at least one processor, a recommendation on a display of the computing device recommending surgical intervention where the cancer risk level is the high risk. In some embodiments, the methods of the invention further include generating, by the at least one processor, a recommendation on a display of the computing device recommending no surgical intervention where the cancer risk level is the low risk.

In some embodiments, the methods of the invention further include receiving, by the at least one processor, a modification to the cancer risk level prediction from the computing device; and retraining, by the at least one processor, the learned risk classification parameters based on a difference between the modification and the cancer risk level. In some embodiments, the first panel signal is received from a mass spectrometer or a biochip or both in communication with the at least one processor. In some embodiments, the first stage cancer risk classifier comprises a classification tree or an artificial neural network. In some embodiments, the first stage cancer risk classifier comprises a supervised classification model. In some embodiments, the first stage cancer risk classifier comprises an unsupervised classification model.

In some embodiments, the subject is diagnosed with an asymptomatic adnexal mass. In some embodiments, the subject is diagnosed with a symptomatic adnexal mass. In some embodiments, the subject is pre-menopausal. In some embodiments, the subject is post-menopausal. In some embodiments, the biological sample from a subject is serum.

In one aspect, the invention provides a system comprising at least one processor configured to execute instructions causing the at least one processor to perform any of the methods as provided herein. In some embodiments, the at least one processor is in communication with a memory having the instructions stored thereon. In some embodiments, the at least one processor is further configured to execute the instructions to perform steps to generate recommendation on a display of the computing device recommending surgical intervention where the cancer risk level is the high risk. In some embodiments, the at least one processor is further configured to execute the instructions to perform steps to generate a recommendation on a display of the computing device recommending no surgical intervention where the cancer risk level is the low risk. In some embodiments, the at least one processor is further configured to execute the instructions to perform steps to: receive a modification to the cancer risk level prediction from the computing device; and retrain the learned risk classification parameters based on a difference between the modification and the cancer risk level. In some embodiments, a system of the invention provided herein comprising a mass spectrometer in communication with the at least one processor.

In one aspect, the invention provides a non-transitory computer readable medium storing thereon software, the software comprising program instructions configured to cause the at least one processor to perform any of the methods provided herein. In some embodiments, the methods of the invention provided herein further include the step of generating a recommendation on a display of the computing device recommending surgical intervention where the cancer risk level is the high risk. In some embodiments, the methods of the invention provided herein further include the step of generating a recommendation on a display of the computing device recommending no surgical intervention where the cancer risk level is the low risk. In some embodiments, the methods of the invention provided herein further include receiving a modification to the cancer risk level prediction from the computing device; and retraining the learned risk classification parameters based on a difference between the modification and the cancer risk level. In some embodiments, the first stage cancer risk classifier comprises a classification tree or an artificial neural network. In some embodiments, the first stage cancer risk classifier comprises a supervised classification model. In some embodiments, the first stage cancer risk classifier comprises an unsupervised classification model.

In one aspect the invention provides a kit comprising: (a) any of the panels of markers of the invention as provided herein; and (b) instructions for using the panel for pre-operatively assessing a subject's risk of having ovarian cancer. In particular embodiments, use of these panels unexpectedly increased specificity, increased sensitivity, and/or reduced the rate of false positives or false negatives identified by conventional panels of biomarkers.

As described in detail herein, any method known in the art can be used to measure a panel of biomarkers. In aspects of the invention, the panel of biomarkers are measured using any immunoassay well known in the art. In embodiments, the immunoassay can be, but is not limited to, ELISA, western blotting, and radioimmunoassay.

Compositions and articles defined by the invention were isolated or otherwise manufactured in connection with the examples provided below. Other features and advantages of the invention will be apparent from the detailed description, and from the claims

### Definitions

Unless defined otherwise, all technical and scientific terms used herein have the meaning commonly understood by a person skilled in the art to which this invention pertains or relates. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); Benjamin Lewin, Genes V, published by Oxford University Press, 1994 (ISBN 0-19-854287-9); Kendrew et al. (eds.); The Encyclopedia of Molecular Biology, published by Blackwell Science Ltd., 1994 (ISBN 0-632-02182-9); Molecular Biology and Biotechnology: a Comprehensive Desk Reference, Robert A. Meyers (ed.), published by VCH Publishers, Inc., 1995 (ISBN 1-56081-569-8); and Hale & Marham, The Harper Collins Dictionary of Biology (1991). As used herein, the following terms have the meanings ascribed to them below, unless specified otherwise.

By “adnexal mass” is meant an abnormal growth that develops near the uterus, most commonly arising from the ovaries, fallopian tubes, or connective tissues. The lump-like mass can be cystic (fluid-filled) or solid. Adnexal masses may be benign (non-cancerous) or malignant (cancerous). Adnexal masses may be symptomatic or asymptomatic. By a “symptomatic adnexal mass” is meant an adnexal mass that presents symptoms in a patient. The symptoms may include, but are not limited to, abdominal fullness, abdominal bloating, pelvic pain, difficulty with bowel movements, and increased frequency of urination, abnormal vaginal bleeding, or pelvic pressure. By “asymptomatic adnexal mass” is meant an adnexal mass producing or showing no symptoms in a patient.

By “agent” is meant any small molecule chemical compound, antibody, nucleic acid molecule, or polypeptide, or fragments thereof.

By “alteration” is meant a change (increase or decrease) in the expression levels or activity of a gene or polypeptide as detected by standard art known methods such as those described herein. An alteration may be by as little as 1%, 2%, 3%, 4%, 5%, 10%, 20%, 30%, or by 40%, 50%, 60%, or even by as much as 70%, 75%, 80%, 90%, or 100%.

By “biologic sample” is meant any tissue, cell, fluid, or other material derived from an organism.

A “biomarker” or “marker” as used herein generally refers to a protein, nucleic acid molecule, clinical indicator, or other analyte that is associated with a disease. In one embodiment, a marker of ovarian cancer is differentially present in a biological sample obtained from a subject having or at risk of developing ovarian cancer relative to a reference. A marker is differentially present if the mean or median level of the biomarker present in the sample is statistically different from the level present in a reference. A reference level may be, for example, the level present in a sample obtained from a healthy control subject or the level obtained from the subject at an earlier timepoint, i.e., prior to treatment. Common tests for statistical significance include, among others, t-test, ANOVA, Kruskal-Wallis, Wilcoxon, Mann-Whitney and odds ratio. Biomarkers, alone or in combination, provide measures of relative likelihood that a subject belongs to a phenotypic status of interest. The differential presence of a marker of the invention in a subject sample can be useful in characterizing the subject as having or at risk of developing ovarian cancer, for determining the prognosis of the subject, for evaluating therapeutic efficacy, or for selecting a treatment regimen (e.g., selecting that the subject be evaluated and/or treated by a surgeon that specializes in gynecologic oncology).

Markers useful in the panels of the invention include, for example, FSH, HE4, CA125, transthyretin, transferrin, ApoA1, and β2 microglobulin proteins, as well as the nucleic acid molecules encoding such proteins. Fragments useful in the methods of the invention are sufficient to bind an antibody that specifically recognizes the protein from which the fragment is derived. The invention includes markers that are substantially identical to the following sequences. Preferably, such a sequence is at least 85%, 90%, 95% or even 99% identical at the amino acid level or nucleic acid to the sequence used for comparison.

As used herein, the terms “comprises,” “comprising,” “containing,” “having” and the like can have the meaning ascribed to them in U.S. patent law and can mean “includes,” “including,” and the like; “consisting essentially of” or “consists essentially” likewise has the meaning ascribed in U.S. patent law and the term is open-ended, allowing for the presence of more than that which is recited so long as basic or novel characteristics of that which is recited is not changed by the presence of more than that which is recited, but excludes prior art embodiments.

By “Follicle-stimulating hormone (FSH) polypeptide” is meant a polypeptide or fragment thereof having at least about 85% amino acid identity to NCBI Accession No. NP_000501.

By “Human Epididymis Protein 4 (HE4) polypeptide” is meant a polypeptide or fragment thereof having at least about 85% amino acid identity to NCBI Accession No. NP_006094.

By “Cancer Antigen 125 (CA125) polypeptide” is meant a polypeptide or fragment thereof having at least about 85% amino acid identity to Swiss-Prot Accession number Q8WXI7.

By “Transthyretin (Prealbumin) polypeptide” is meant a polypeptide or fragment thereof having at least about 85% amino acid identity to Swiss Prot Accession number P02766.

By “Transferrin polypeptide” is meant a polypeptide or fragment thereof having at least about 85% amino acid identity to UniProtKB/TrEMBL Accession number Q06AH7.

By “Apolipoprotein A1 (ApoA1) polypeptide” is meant a polypeptide or fragment thereof having at least about 85% amino acid identity to Swiss Prot Accession number P02647.

By “β-2 microglobulin polypeptide” is meant a polypeptide or fragment thereof having at least about 85% amino acid identity to SwissProt Accession No. P61769.

By “Breast cancer 1 (BRCA1) gene” is meant a gene on chromosome 17 that normally helps to suppress cell growth having at least about 85% nucleotide identity to NCBI Accession No. NG_005905.2. Mutations in the BRCA1 gene are associated with a higher risk of breast, ovarian, prostate, and other types of cancer.

By “Breast cancer 1 (BRCA1) polypeptide” is meant a polypeptide or fragment thereof having at least about 85% amino acid identity to GenBank Accession No. AAC37594.1.

By “Breast cancer 2 (BRCA2) gene” is meant a gene on chromosome 13 that normally helps to suppress cell growth having at least about 85% nucleotide identity to NCBI Accession No. NG_012772.3. Mutations in the BRCA2 gene are associated with a higher risk of breast, ovarian, prostate, and other types of cancer.

By “Breast cancer 2 (BRCA2) polypeptide” is meant a polypeptide or fragment thereof having at least about 85% amino acid identity to NCBI Accession No. NP_000050.2.

By “Ataxia-Telangiesctasia mutated (ATM) gene” is meant a gene on chromosome 11 that encodes a serine/threonine protein kinase that is recruited and activated by DNA double-strand breaks to phosphorylate proteins that initiate activation of the DNA damage checkpoint, leading to cell cycle arrest, DNA repair or apoptosis and having at least about 85% nucleotide identity to NCBI Reference Sequence: NG_009830.1. Mutations in the ATM gene are associated with a higher risk of breast, ovarian, prostate, pancreatic and other types of cancer.

By “Ataxia-Telangiesctasia mutated (ATM) polypeptide” is meant a polypeptide or fragment thereof having at least about 85% amino acid identity to NCBI Reference Sequence: NP_000042.3.

By “BRCA1 Associated Ring Domain 1 (BARD1) gene” is meant a gene on chromosome 2 that encodes a protein that heterodimerizes with BRCA1 via N-terminal Ring finger domains to stabilize BRCA1 and having at least about 85% nucleotide identity to NCBI Reference Sequence: NG_012047.3. Mutations in BARD1 that affect protein structure as associated with breast, ovarian, and uterine cancers, suggesting the mutations disable BARD1's tumor suppressor function.

By “BRCA1 Associated Ring Domain 1 (BARD1) polypeptide” is meant a polypeptide or fragment thereof having at least about 85% amino acid identity to NCBI Reference Sequence: NP_000456.2.

By “BRCA1 Interacting Protein C-terminal Helicase 1 (BRIP1) gene” is meant a gene that encodes the Fanconi anemia group J protein, a member of the RecQ DEAH helicase family (“DEAH” disclosed as SEQ ID NO: 1), which interacts with BRCA1 and having at least about 85% nucleotide identity to NCBI Reference Sequence: NG_007409.2. Mutations in BRIP1 are associated with ovarian cancer.

By “BRCA1 Interacting Protein C-terminal Helicase 1 (BRIP1) polypeptide” is meant a polypeptide or fragment thereof having at least about 85% amino acid identity to NCBI Reference Sequence: NP_114432.2.

By “Cadherin-1 (CDH1) gene” is meant a gene on chromosome 16 that encodes a calcium-dependent cell-cell adhesion glycoprotein and having at least about 85% nucleotide identity to NCBI Reference Sequence: NG_008021.1. Mutations in the CDH1 gene are associated with gastric, breast, colorectal, thyroid, and ovarian cancers.

By “Cadherin-1 (CDH1) polypeptide” is meant a polypeptide or fragment thereof having at least about 85% amino acid identity to NCBI Reference Sequence: NP_004351.1.

By “Checkpoint Kinase 2 (CHEK2) gene” is meant a gene pm chromosome 22 that encodes a serine-threonine kinase, which is involved in DNA repair, cell cycle arrest or apoptosis in response to DNA damage and having at least about 85% nucleotide identity to NCBI Reference Sequence: NG_008150.2. Mutations in the CHEK2 gene are associated with breast, prostate, lunch, colon, kidney and thyroid cancers.

By “Checkpoint Kinase 2 (CHEK2) polypeptide” is meant a polypeptide or fragment thereof having at least about 85% amino acid identity to NCBI Reference Sequence: NP_009125.1.

By “Epithelial cell adhesion molecule (EPCAM) gene” is meant a gene on chromosome 2 encoding a transmembrane glycoprotein mediating Ca2+-independent homotypic cell-cell adhesion in epithelia, cell signaling, migration, proliferation, and differentiation and having at least about 85% nucleotide identity to NCBI Reference Sequence: NG_012352.2. Mutations in the EPCAM gene are associated with several cancers, including breast and ovarian cancer.

By “Epithelial cell adhesion molecule (EPCAM) polypeptide” is meant a polypeptide or fragment thereof having at least about 85% amino acid identity to NCBI Reference Sequence: NP_002345.2.

By “MutL homolog 1 (MLH1) gene” is meant a gene on chromosome 3 encoding a DNA mismatch repair protein and having at least about 85% nucleotide identity to NCBI Reference Sequence: NG_007109.2. Mutations in the MLH1 gene are associated with colon, endometrial, and ovarian cancers.

By “MutL homolog 1 (MLH1) polypeptide” is meant a polypeptide or fragment thereof having at least about 85% amino acid identity to NCBI Reference Sequence: NP_000240.1.

By “MutS Homolog 2 (MSH2) gene” is meant a gene on chromosome 2 encoding a DNA mismatch repair protein and having at least about 85% nucleotide identity to NCBI Reference Sequence: NG_007110.2. Mutations in the MSH2 gene are associated with colon, breast, and ovarian cancers.

By “MutS Homolog 2 (MSH2) polypeptide” is meant a polypeptide or fragment thereof having at least about 85% amino acid identity to NCBI Reference Sequence: NP_000242.1.

By “MutS Homolog 6 (MSH6) gene” is meant a gene on chromosome 2 encoding a protein involved in DNA repair and having at least about 85% nucleotide identity to NCBI Reference Sequence: NG_007111.1. Mutations in the MSH6 gene are associated with colon, endometrial, breast and ovarian cancers.

By “MutS Homolog 6 (MSH6) polypeptide” is meant a polypeptide or fragment thereof having at least about 85% amino acid identity to NCBI Reference Sequence: NP_000170.1.

By “Nibrin (NBN) gene” is meant a gene on chromosome 6 encoding a DNA mismatch repair protein and having at least about 85% nucleotide identity to NCBI Reference Sequence: NG_008860.1. Mutations in the NBN gene are associated with breast, prostate and ovarian cancers.

By “Nibrin (NBN) polypeptide” is meant a polypeptide or fragment thereof having at least about 85% amino acid identity to NCBI Reference Sequence: NP_002476.2.

By “partner and localizer of BRCA2 (PALB2) gene” is meant a gene on chromosome 16 encoding a protein involved in double strand break repair and binds to and colocalizes with BRCA 2 and having at least about 85% nucleotide identity to NCBI Reference Sequence: NG_007406.1. Mutations in the PALB2 gene are associated with ovarian, breast and pancreatic cancers.

By “partner and localizer of BRCA2 (PALB2) polypeptide” is meant a polypeptide or fragment thereof having at least about 85% amino acid identity to NCBI Reference Sequence: NP_078951.2.

By “Phosphatase and tensin homolog (PTEN) gene” is meant a gene on chromosome 10 encoding a phosphatase protein involved in cell cycle regulation and having at least about 85% nucleotide identity to NCBI Reference Sequence: NG_007466.2. Mutations in the PTEN gene are associated with prostate, breast and ovarian cancers.

By “Phosphatase and tensin homolog (PTEN) polypeptide” is meant a polypeptide or fragment thereof having at least about 85% amino acid identity to NCBI Reference Sequence: NP_000305.3.

By “RAD51 paralog D (RAD51D) gene” is meant a gene on chromosome 17 encoding a protein involved in the homologous recombination and repair of DNA and having at least about 85% nucleotide identity to NCBI Reference Sequence: NG_031858.1. Mutations in the RAD51D gene are associated with breast and ovarian cancers.

By “RAD51 paralog D (RAD51D) polypeptide” is meant a polypeptide or fragment thereof having at least about 85% amino acid identity to NCBI Reference Sequence: NP_002869.3.

By “Serine/Threonine Kinase 11 (STK11) gene” is meant a gene on chromosome 19 encoding a serine/threonine protein kinase and having at least about 85% nucleotide identity to NCBI Reference Sequence: NG_007460.2. Mutations in the STK11 gene are associated with ovarian, cervical, breast, intestinal, testicular, pancreatic and skin cancers.

By “Serine/Threonine Kinase 11 (STK11) polypeptide” is meant a polypeptide or fragment thereof having at least about 85% amino acid identity to NCBI Reference Sequence: NP_000446.1.

By “Tumor protein p53 (TP53) gene” is meant a gene on chromosome 17 encoding a tumor suppressor protein and having at least about 85% nucleotide identity to NCBI Reference Sequence: NG_017013.2. Mutations in the TP53 gene are associated with a variety of cancers, including breast and ovarian cancer.

By “Tumor protein p53 (TP53) polypeptide” is meant a polypeptide or fragment thereof having at least about 85% amino acid identity to NCBI Reference Sequence: NP_000537.3.

By “Kirsten rat sarcoma viral oncogene homolog (KRAS) gene” is meant a gene on chromosome 12 encoding a GTPase involved in cell signaling and having at least about 85% nucleotide identity to NCBI Reference Sequence: NG_007524.2. Mutations in the KRAS gene are associated with a variety of cancers, including colon, lung, ovarian and breast cancers.

By “Kirsten rat sarcoma viral oncogene homolog (KRAS) polypeptide” is meant a polypeptide or fragment thereof having at least about 85% amino acid identity to NCBI Reference Sequence: NP_203524.1.

Select exemplary sequences delineated herein are shown in FIG. 1.

By “capture reagent” is meant a reagent that specifically binds a nucleic acid molecule or polypeptide to select or isolate the nucleic acid molecule or polypeptide.

By “clinical aggressiveness” is meant the severity of the neoplasia. Aggressive neoplasias are more likely to metastasize than less aggressive neoplasias. While conservative methods of treatment are appropriate for less aggressive neoplasias, more aggressive neoplasias require more aggressive therapeutic regimens.

As used herein, the terms “determining,” “assessing,” “assaying,” “measuring” and “detecting” refer to both quantitative and qualitative determinations of an analyte, and as such, the term “determining” is used interchangeably herein with “assaying,” “measuring,” and the like. Where a quantitative determination is intended, the phrase “determining an amount” of an analyte and the like is used. Where a qualitative and/or quantitative determination is intended, the phrase “determining a level” of an analyte or “detecting” an analyte is used.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that damages or interferes with the normal function of a cell, tissue, or organ. Examples of diseases include breast and ovarian cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The invention provides a number of targets that are useful for the development of highly specific drugs to treat or a disorder characterized by the methods delineated herein. In addition, the methods of the invention provide a facile means to identify therapies that are safe for use in subjects. In addition, the methods of the invention provide a route for analyzing virtually any number of compounds for effects on a disease described herein with high-volume throughput, high sensitivity, and low complexity.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

By “germline marker” is meant any protein or polynucleotide within germ cells having an alteration in expression level or activity that is associated with a disease or disorder that can be passed on to offspring.

By “germline mutation” is meant an inherited genetic alteration within germ cells.

“Hybridization” means hydrogen bonding, which may be Watson-Crick, Hoogsteen or reversed Hoogsteen hydrogen bonding, between complementary nucleobases. For example, adenine and thymine are complementary nucleobases that pair through the formation of hydrogen bonds.

The terms “isolated,” “purified,” or “biologically pure” refer to material that is free to varying degrees from components which normally accompany it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide of this invention is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high-performance liquid chromatography. The term “purified” can denote that a nucleic acid or protein gives rise to essentially one band in an electrophoretic gel. For a protein that can be subjected to modifications, for example, phosphorylation or glycosylation, different modifications may give rise to different isolated proteins, which can be separately purified.

By “isolated biomarker” or “purified biomarker” is meant at least 60%, by weight, free from proteins and naturally-occurring organic molecules with which the marker is naturally associated. Preferably, the preparation is at least 75%, more preferably 80, 85, 90 or 95% pure or at least 99%, by weight, a purified isolated biomarker.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide of the invention that has been separated from components that naturally accompany it. Typically, the polypeptide is isolated when it is at least 60%, by weight, free from the proteins and naturally-occurring organic molecules with which it is naturally associated. Preferably, the preparation is at least 75%, more preferably at least 90%, and most preferably at least 99%, by weight, a polypeptide of the invention. An isolated polypeptide of the invention may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

By “marker profile” is meant a characterization of the expression or expression level of two or more polypeptides or polynucleotides.

By “neoplasia” is meant any disease that is caused by or results in inappropriately high levels of cell division, inappropriately low levels of apoptosis, or both. Examples of cancers include, without limitation, prostate cancer, leukemias (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (Hodgkin's disease, non-Hodgkin's disease), Waldenstrom's macroglobulinemia, heavy chain disease, and solid tumors such as sarcomas and carcinomas (e.g., fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, breast cancer, ovarian cancer, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, nile duct carcinoma, choriocarcinoma, seminoma, embryonal carcinoma, Wilm's tumor, cervical cancer, uterine cancer, testicular cancer, lung carcinoma, small cell lung carcinoma, bladder carcinoma, epithelial carcinoma, glioma, astrocytoma, medulloblastoma, craniopharyngioma, ependymoma, pinealoma, hemangioblastoma, acoustic neuroma, oligodenroglioma, schwannoma, meningioma, melanoma, neuroblastoma, and retinoblastoma). Lymphoproliferative disorders are also considered to be proliferative diseases.

As used herein, “obtaining” as in “obtaining an agent” includes synthesizing, purchasing, or otherwise acquiring the agent.

The term “ovarian cancer” refers to both primary ovarian tumors as well as metastases of the primary ovarian tumors that may have settled anywhere in the body.

The term “ovarian cancer status” refers to the status of the disease in the patient. Examples of types of ovarian cancer statuses include, but are not limited to, the subject's risk of cancer, the presence or absence of disease, the stage of disease in a patient, and the effectiveness of treatment of disease. In embodiments, a subject identified as having a pelvic mass is assessed to identify if their ovarian cancer status is benign or malignant.

Nucleic acid molecules useful in the methods of the invention include any nucleic acid molecule that encodes a polypeptide of the invention or a fragment thereof. Such nucleic acid molecules need not be 100% identical with an endogenous nucleic acid sequence, but will typically exhibit substantial identity. Polynucleotides having “substantial identity” to an endogenous sequence are typically capable of hybridizing with at least one strand of a double-stranded nucleic acid molecule. By “hybridize” is meant pair to form a double-stranded molecule between complementary polynucleotide sequences (e.g., a gene described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 μg/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42° C. in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

By “reduces” is meant a negative alteration. In some embodiments, the alteration is reduced by at least 5%, 10%, 25%, 50%, 75%, or 100%.

By “reference” is meant a standard or control condition of comparison. For example, the marker level(s) present in a patient sample may be compared to the level of the marker in a corresponding healthy cell or tissue or in a diseased cell or tissue (e.g., a cell or tissue derived from a subject having ovarian cancer). In particular embodiments, the IGFBP2, IL6, FSH, HE4, CA125; Transthyretin, Transferrin, TAG-72/CA 72-4 polypeptide level present in a patient sample may be compared to the level of said polypeptide present in a corresponding sample obtained at an earlier time point (i.e., prior to treatment), to a healthy cell or tissue or a neoplastic cell or tissue that lacks a propensity to metastasize.

By “sample” is meant a biologic sample such as any tissue, cell, fluid, or other material derived from an organism.

“Sequence identity” refers to the similarity between amino acid or nucleic acid sequences that is expressed in terms of the similarity between the sequences. Sequence identity is frequently measured in terms of percentage identity (or similarity or homology); the higher the percentage, the more similar the sequences are. Homologs or variants of a given gene or protein will possess a relatively high degree of sequence identity when aligned using standard methods. Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence. In addition, other programs and alignment algorithms are described in, for example, Smith and Waterman, 1981, Adv. Appl. Math. 2:482; Needleman and Wunsch, 1970, J Mol. Biol. 48:443; Pearson and Lipman, 1988, Proc. Natl. Acad. Sci. U.S.A. 85:2444; Higgins and Sharp, 1988, Gene 73:237-244; Higgins and Sharp, 1989, CABIOS 5:151-153; Corpet et al., 1988, Nucleic Acids Research 16:10881-10890; Pearson and Lipman, 1988, Proc. Natl. Acad. Sci. U.S.A. 85:2444; and Altschul et al., 1994, Nature Genet. 6:119-129. The NCBI Basic Local Alignment Search Tool (BLAST™) (Altschul et al. 1990, J. Mol. Biol. 215:403-410) is readily available from several sources, including the National Center for Biotechnology Information (NCBI, Bethesda, Md.) and on the Internet, for use in connection with the sequence analysis programs blastp, blastn, blastx, tblastn and tblastx.

By “somatic marker” is meant any protein or polynucleotide having an alteration in expression level or activity that is associated with a disease or disorder that can occur within any cell except germ cells and is not inheritable.

By “somatic mutation” is meant a genetic alteration within any cell except germ cells and is not inheritable.

By “specifically binds” is meant a compound (e.g., antibody) that recognizes and binds a molecule (e.g., polypeptide), but which does not substantially recognize and bind other molecules in a sample, for example, a biological sample.

The accuracy of a diagnostic test can be characterized using any method well known in the art, including, but not limited to, a Receiver Operating Characteristic curve (“ROC curve”). An ROC curve shows the relationship between sensitivity and specificity. Sensitivity is the percentage of true positives that are predicted by a test to be positive, while specificity is the percentage of true negatives that are predicted by a test to be negative. An ROC is a plot of the true positive rate against the false positive rate for the different possible cutpoints of a diagnostic test. Thus, an increase in sensitivity will be accompanied by a decrease in specificity. The closer the curve follows the left axis and then the top edge of the ROC space, the more accurate the test. Conversely, the closer the curve comes to the 45-degree diagonal of the ROC graph, the less accurate the test. The area under the ROC is a measure of test accuracy. The accuracy of the test depends on how well the test separates the group being tested into those with and without the disease in question. An area under the curve (referred to as “AUC”) of 1 represents a perfect test. In embodiments, biomarkers and diagnostic methods of the present invention have an AUC greater than 0.50, greater than 0.60, greater than 0.70, greater than 0.80, or greater than 0.90.

Other useful measures of the utility of a test are positive predictive value (“PPV”) and negative predictive value (“NPV”). PPV is the percentage of actual positives who test as positive. NPV is the percentage of actual negatives that test as negative.

The term “subject” or “patient” refers to an animal which is the object of treatment, observation, or experiment. By way of example only, a subject includes, but is not limited to, a mammal, including, but not limited to, a human or a non-human mammal, such as a non-human primate, murine, bovine, equine, canine, ovine, or feline.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, more preferably 80% or 85%, and more preferably 90%, 95% or even 99% identical at the amino acid level or nucleic acid to the sequence used for comparison.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

Any compounds, compositions, or methods provided herein can be combined with one or more of any of the other compositions and methods provided herein.

As used herein, the singular forms “a”, “an”, and “the” include plural forms unless the context clearly dictates otherwise. Thus, for example, reference to “a biomarker” includes reference to more than one biomarker.

Unless specifically stated or obvious from context, as used herein, the term “or” is understood to be inclusive.

The term “including” is used herein to mean, and is used interchangeably with, the phrase “including but not limited to.”

Any compositions or methods provided herein can be combined with one or more of any of the other compositions and methods provided herein.

### BRIEF DESCRIPTION OF THE DRAWINGS

FIG. 1 provides exemplary sequences of Follicle-stimulating hormone (FSH); Human Epididymis Protein 4 (HE4); Cancer Antigen 125 (CA 125); Transthyretin (prealbumin); Transferrin; apolipoprotein A-1 (ApoA1), β2-microglobulin (β2M), BRCA1, and BRCA2 polypeptides. FIG. 1 discloses SEQ ID NOS 2-26, respectively, in order of appearance.

FIG. 2 provides study demographics and clinicopathologic information according to menopausal stage.

FIGS. 3A-3C are graphs depicting the receiver operating characteristic (ROC) curves of AMRA with comparison to CA125 on pre-menopausal (FIG. 3A, left), post-menopausal (FIG. 3B, middle), and pre-menopausal, stage I/II invasive cancer (FIG. 3C, right) patients and benign adnexal masses only in a training set (OVA1) and three validation sets (OVA500, FHCRC #7788 and OVA1-PS1-CO4) (AUC: Area under curve; ROC: Receiver operating characteristic).

FIGS. 4A-4B depict the distributions of benign, low-malignant potential tumor stage I/II and stage III/IV patients in adnexal mass risk assessment (AMRA) risk groups (HR: High risk; IR: Intermediate risk; LR: Lower risk). FIG. 3A is a bar chart depicting the risk of malignancy compared to AMRA in pre-menopausal patients adjusted for an assumed pre-test prevalence at 5%. FIG. 3B is a bar chart depicting the risk of malignancy compared to AMRA in post-menopausal patients adjusted for an assumed pre-test prevalence at 10%.

FIG. 5 provides the distribution of benign, low-malignant potential tumor/early stage, late stage cancer in pre-menopausal adnexal mass risk assessment risk groups (actual and projected based on assumed prevalence).

FIG. 6 provides the distribution of benign, low-malignant potential tumor/early stage, late stage cancer in post-menopausal adnexal mass risk assessment risk groups (actual and projected based on assumed prevalence).

FIGS. 7A-7B depict prevalence-adjusted post-test cancer probabilities of the adnexal mass risk assessment risk groups (HR: High risk; IR: Intermediate risk; LR: Lower risk) projected based on the training set (OVA1) and combined validation datasets (OVA500, FHCRC #7788 and OVA1-PS1-CO4). The estimated cancer probability bars were superimposed with an interpolation curve by logistic regression. FIG. 7A provides bar charts depicting probabilities in pre-menopausal patients. FIG. 7B provides bar charts depicting probabilities in post-menopausal patients.

FIG. 8 provides a table depicting the estimated performance metrics of adnexal mass risk assessment groups.

FIG. 9 provides a flow chart depicting the categorization of the sample sets used for validation of the adnexal mass risk assessment groups.

### DETAILED DESCRIPTION OF THE INVENTION

The invention comprises panels of biomarkers and the use of such panels for pre-operatively assessing a subject's risk of having ovarian cancer. The invention is based, at least in part, on the discovery that panels of the invention advantageously enhance specificity (e.g., to about mean/median 70%, 75%, 80%, 85%, 90%) and sensitivity (e.g., to at least 75%) and reduce false positives and false negatives identified by conventional panels of biomarkers for pre- and post-menopausal subjects diagnosed with an adnexal mass (e.g., symptomatic or asymptomatic).

In particular, the invention provides panels comprising or consisting of the following sets of markers:

Apolipoprotein A1 (ApoA1), Cancer Antigen 125 (CA125), β2 microglobulin (β2M), Transferrin (Tfr), and Transthyretin/prealbumin (TT);

Follicle-stimulating hormone (FSH), CA125, Human Epididymis Protein 4 (HE4), ApoA1, and Transferrin;

ApoA1, CA125, β2M, Transferrin, TT, FSH, and HE4;

ApoA1, CA125, β2M, Transferrin, TT, and Breast Cancer 1 (BRCA1);

FSH, CA125, HE4, ApoA1, Transferrin, and BRCA1;

ApoA1, CA125, β2M, Transferrin, TT, and Breast Cancer 2 (BRCA2);

FSH, CA125, HE4, ApoA1, Transferrin, and BRCA2;

ApoA1, CA125, β2M, Transferrin, TT, BRCA1, and BRCA2;

FSH, CA125, HE4, ApoA1, Transferrin, BRCA1, and BRCA2;

ApoA1, CA125, β2M, Transferrin, TT, FSH, HE4, and BRCA1;

ApoA1, CA125, β2M, Transferrin, TT, FSH, HE4, and BRCA2; and

ApoA1, CA125, β2M, Transferrin, TT, FSH, HE4, BRCA1, and BRCA2.

Additionally, the invention is based, at least in part, on the discovery that the characterization of additional germline and/or somatic mutations to multivariate index assays (e.g., AMRA, OVA1, and/or OVERA) enhances specificity and sensitivity and reduces false positives and false negatives identified by conventional panels of biomarkers for pre- and post-menopausal subjects diagnosed with an adnexal mass. Similarly, the presence of aberrant methylation (e.g., hypermethylation or hypomethylation) of biomarkers enhances specificity and sensitivity and reduces false positives and false negatives identified by conventional panels of biomarkers for pre- and post-menopausal subjects diagnosed with an adnexal mass.

In some embodiments, the above sets of markers may be combined with one or more of the following markers associated with breast and/or ovarian cancer: Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P161NK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1).

The methods of the invention also include reducing the rate of false positive or reducing the rate of false negative pre-operative ovarian cancer assessment in pre- and post-menopausal subjects. The invention further features the use of such panels for pre-operatively assessing a subject's risk of having ovarian cancer. In particular, the use of such panels provides methods for pre-surgically characterizing a subject (e.g., pre- or post-menopausal) diagnosed with an adnexal mass (e.g., symptomatic or asymptomatic) as having a high or low risk of cancer.

### Ovarian Cancer

Ovarian tumors are being detected with increasing frequency in women of all ages, yet there is no standardized or reliable method to determine which are malignant prior to surgery. In 1994, the National Institutes of Health (NIH) released a consensus statement indicating that women with ovarian masses having been identified preoperatively as having a significant risk of ovarian cancer should be given the option of having their surgery performed by a gynecologic oncologist. At present, the National Comprehensive Cancer Network (NCCN), the Society of Gynecologic Oncologists (SGO), SOGC clinical practice guidelines, Standing Subcommittee on Cancer of the Medical Advisory Committee, and several other published statements, all recommend that women with ovarian cancer be under the care of a gynecologic oncologist (GO).

Recent publications on breast, bladder, gastrointestinal, and ovarian cancers have reported improved outcome when cancer management involves a surgical specialist. In addition, a recent meta-analysis of 18 ovarian cancer studies found that the early involvement of a gynecologic oncologist, rather than a general surgeon or general gynecologist, improved patient outcomes. The authors concluded: 1) subjects with early stage disease are more likely to have comprehensive surgical staging, facilitating appropriate adjuvant chemotherapy, 2) subjects with advanced disease are more likely to receive optimal cytoreductive surgery, and 3) subjects with advanced disease have an improved median and overall 5-year survival. Despite the availability of this important information, only a fraction of women with malignant ovarian tumors (an estimated 33%) are referred to a gynecologic oncologist for the primary surgery. Based on reported patterns of care for ovarian cancer management, the majority of women in the United States may not be receiving optimal care for this disease.

The decision for operative removal of an ovarian tumor, and whether a generalist or specialist should perform the surgery, is based on interpretations of physical examination, imaging studies, laboratory tests, and clinical judgment. Pelvic examination alone is inadequate to reliably detect or differentiate ovarian tumors, particularly in early stages when ovarian cancer treatment is most successful. Examination has also been eliminated from the Prostate, Lung, Colorectal and Ovarian cancer screening trial algorithm. Pelvic ultrasound is clinically useful and the least expensive imaging modality, but has limitations in consistently identifying malignant tumors. In general, nearly all unilocular cysts are benign, whereas complex cystic tumors with solid components or internal papillary projections are more likely to be malignant. CA125 has been used alone or in conjunction with other tests in an effort to establish risk of malignancy. Unfortunately, CA125 has low sensitivity (50%) in early stage ovarian cancers, and low specificity resultant from numerous false positives in both pre- and postmenopausal women.

The American College of Obstetrics and Gynecology (ACOG) and the SGO have published referral guidelines for patients with a pelvic mass. These guidelines include: patient age, serum CA125 level, physical examination, imaging results, and family history. This referral strategy has been evaluated both retrospectively and prospectively. In a single institution review, Dearking and colleagues concluded that the guidelines were useful in predicting advanced stage ovarian cancer, but “performed poorly in identifying early-stage disease, especially in premenopausal women, primarily due to lack of early markers and signs of ovarian cancer”.

### Biomarkers

In particular embodiments, a biomarker is an organic biomolecule that is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for characterizing a disease.

### Biomarkers for Ovarian Cancer

The invention provides a panel of polypeptide or polynucleotide biomarkers that are differentially present in subjects having ovarian cancer, in particular, a benign vs. malignant pelvic mass. The biomarkers of this invention are differentially present depending on ovarian cancer status, including subjects having ovarian cancer vs. subjects that do not have ovarian cancer, or menopausal status, including subjects that are pre- or post-menopausal.

The biomarker panel of the invention comprises one or more of the biomarkers presented in the following Table 1.

TABLE 1

| | Differential Regulation |
|---|---|
| Biomarker | in ovarian cancer |
| ApoA1 | Decreased |
| Beta2 Microglobulin (B2M) | Increased |
| Insulin-like growth factor | Increased |
| binding protein (IGFBP2) | |
| Follicle-stimulating | Increased |
| hormone (FSH) | |
| Human Epididymis | Increased |
| Protein 4 (HE4) | |
| Cancer Antigen 125 (CA125) | Increased |
| Transthyretin (prealbumin) | Decreased |
| Transferrin | Decreased |

As would be understood, references herein to a biomarker of Table 1, a panel of biomarkers, or other similar phrase indicates one or more of the biomarkers set forth in Table 1 or otherwise described herein. A panel of one or more of the biomarkers of Table 1 may be used in combination with one or more panels of one or more of the biomarkers of Table 1. For example, in one embodiment, a panel comprising biomarkers ApoA1, CA125, β2M, Transferrin, TT, FSH, and HE4 may be used in combination with a panel comprising ApoA1, CA125, β2M, Transferrin, and TT. In one embodiment, a panel comprising biomarkers ApoA1, CA125, β2M, Transferrin, TT, FSH, and HE4 may be used in combination with a panel comprising Follicle-stimulating hormone FSH, CA125, HE4, ApoA1, and Transferrin. In one embodiment, a panel comprising biomarkers ApoA1, CA125, β2M, Transferrin, TT, FSH, and HE4 may be used in combination with a panel comprising ApoA1, CA125, β2M, Transferrin, and TT and a panel comprising Follicle-stimulating hormone FSH, CA125, HE4, ApoA1, and Transferrin. The biomarkers of the invention may also include hereditary germline markers (e.g., BRCA1/2) and/or somatic markers that are associated with breast and/or ovarian cancer, including, but not limited to Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P161NK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1). A biomarker of the invention may be detected in a biological sample of the subject (e.g., tissue, fluid), including, but not limited to, blood, blood serum, plasma, saliva, urine, ascites, cyst fluid, a homogenized tissue sample (e.g., a tissue sample obtained by biopsy or liquid biopsy), a cell isolated from a patient sample, and the like.

The invention provides panels comprising isolated biomarkers. The biomarkers can be isolated from biological fluids, such as urine or serum. They can be isolated by any method known in the art. In certain embodiments, this isolation is accomplished using the mass and/or binding characteristics of the markers. For example, a sample comprising the biomolecules can be subject to chromatographic fractionation and subject to further separation by, e.g., acrylamide gel electrophoresis. Knowledge of the identity of the biomarker also allows their isolation by immunoaffinity chromatography. By “isolated biomarker” is meant at least 60%, by weight, free from proteins and naturally-occurring organic molecules with which the marker is naturally associated. Preferably, the preparation is at least 75%, more preferably 80, 85, 90 or 95% pure or at least 99%, by weight, a purified isolated biomarker.

### Follicle-Stimulating Hormone (FSH)

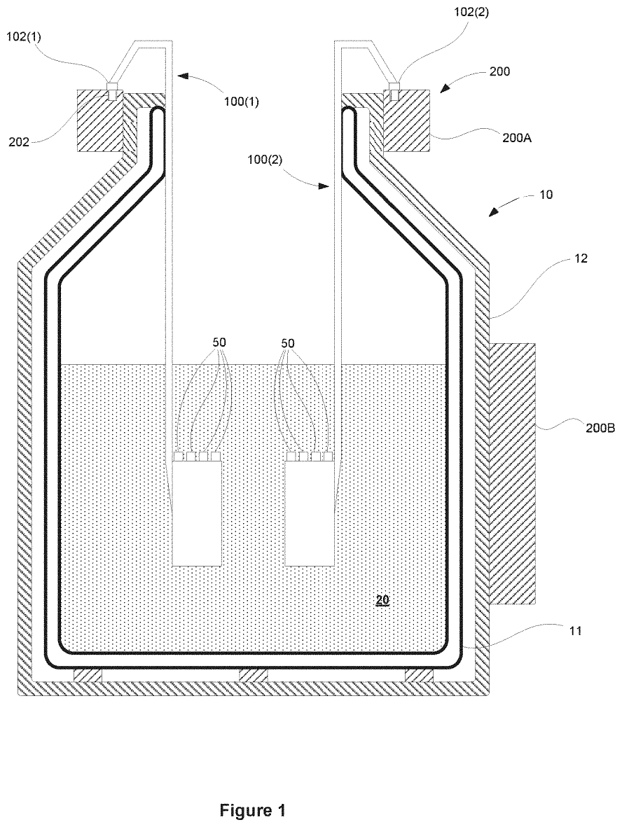

One exemplary biomarker present in the panel of the invention is FSH. FSH is a 128 amino acid protein (NCBI Accession number NP_000501). The amino acid sequence of an exemplary FSH polypeptide is set forth in FIG. 1. Antibodies to FSH can be made using any method well known in the art, or can be purchased from, for example, Santa Cruz Biotechnology, Inc. (e.g., Catalog Number sc-57149) (www.scbt.com, Santa Cruz, Calif.). In aspects of the invention, FSH is upregulated in subjects with ovarian cancer as compared to subjects that do not have ovarian cancer.

### Human Epididymis Protein 4 (HE4)

One exemplary biomarker present in the panel of the invention is HE4. HE4 is a 124 amino acid protein (NCBI Accession number NP_006094). The amino acid sequence of an exemplary HE4 polypeptide is set forth in FIG. 1. Antibodies to HE4 can be made using any method well known in the art, or can be purchased from, for example, Santa Cruz Biotechnology, Inc. (Catalog Number sc-27570) (www.scbt.com, Santa Cruz, Calif.). In aspects of the invention, HE4 is upregulated in subjects with ovarian cancer as compared to subjects that do not have ovarian cancer.

### Cancer Antigen 125 (CA125)

One exemplary biomarker present in the panel of the invention is CA125. CA125 is a 22152 amino acid protein (Swiss-Prot Accession number Q8WXI7). The amino acid sequence of an exemplary CA125 polypeptide is set forth in FIG. 1. Antibodies to CA125 can be made using any method well known in the art, or can be purchased from, for example, Santa Cruz Biotechnology, Inc. (Catalog Number sc-52095) (www.scbt.com, Santa Cruz, Calif.). In aspects of the invention, CA125 is upregulated in subjects with ovarian cancer as compared to subjects that do not have ovarian cancer.

### Transthyretin (Prealbumin)

Another exemplary biomarker present in the panel of the invention is a form of prealbumin, also referred to herein as transthyretin. Transthyretin is a 147 amino acid protein (Swiss Prot Accession number P02766). The amino acid sequence of an exemplary transthyretin polypeptide is set forth in FIG. 1. Antibodies to transthyretin can be made using any method well known in the art, or can be purchased from, for example, Santa Cruz Biotechnology, Inc. (Catalog Number sc-13098) (www.scbt.com, Santa Cruz, Calif.). In aspects of the invention, transthyretin is downregulated in subjects with ovarian cancer as compared to subjects that do not have ovarian cancer.

### Transferrin

Transferrin is another exemplary biomarker of the panel of biomarkers of the invention. Transferrin is a 698 amino acid protein (UniProtKB/TrEMBL Accession number Q06AH7). The amino acid sequence of an exemplary transferring polypeptide is set forth in FIG. 1. Antibodies to transferrin can be made using any method well known in the art, or can be purchased from, for example, Santa Cruz Biotechnology, Inc. (Catalog Number sc-52256) (www.scbt.com, Santa Cruz, Calif.). In aspects of the invention, transferrin is downregulated in subjects with ovarian cancer as compared to subjects that do not have ovarian cancer.

### Apolipoprotein A1

Apolipoprotein A1, also referred to herein as “ApoA1,” is another exemplary biomarker in the panel of biomarkers of the invention. ApoA1 is a 267 amino acid protein (Swiss Prot Accession number P02647). The amino acid sequence of an exemplary ApoA1 is set forth in FIG. 1. Antibodies to Apolipoprotein A1 can be made using any method well known in the art, or can be purchased from, for example, Santa Cruz Biotechnology, Inc. (Catalog Number sc-130503) (www.scbt.com, Santa Cruz, Calif.). In aspects of the invention, ApoA1 is downregulated in subjects with ovarian cancer as compared to subjects that do not have ovarian cancer.

### β2 Microglobulin

One exemplary biomarker that is useful in the methods of the present invention is β2-microglobulin. β2-microglobulin is described as a biomarker for ovarian cancer in US provisional patent publication 60/693,679, filed Jun. 24, 2005 (Fung et al.). The mature form of β2-microglobulin is a 99 amino acid protein derived from an 119 amino acid precursor (GI:179318; SwissProt Accession No. P61769). The amino acid sequence of an exemplary β-2-microglobulin polypeptide is set forth in FIG. 1. The mature form of β-2-microglobulin consist of residues 21-119 of the β-2-microglobulin set forth in FIG. 1. β2-microglobulin is recognized by antibodies. Such antibodies can be made using any method well known in the art, and can also be commercially purchased from, e.g., Abcam (catalog AB759) (www.abcam.com, Cambridge, Mass.). In aspects of the invention, β2-microglobulin is upregulated in subjects with ovarian cancer as compared to subjects that do not have ovarian cancer.

### BRCA1

BRCA1 is another exemplary marker for use in a panel of biomarkers of the invention. The BRCA1 gene is on chromosome 17 and is 193,689 bp (NCBI Accession No. NG_005905.2). The BRCA1 protein is 1863 amino acids (GenBank Accession No. AAC37594.1) and is a part of a complex that repairs double-stranded breaks in DNA and normally helps to suppress cell growth. The amino acid sequence of an exemplary BRCA1 protein is set forth in FIG. 1. Antibodies to BRCA1 can be made using any method well known in the art, or can be purchased from, for example, Santa Cruz Biotechnology, Inc. (Catalog Number sc-6954) (www.scbt.com, Santa Cruz, Calif.).

Germline mutations in the BRCA1 gene are associated with a higher risk of breast, ovarian, prostate, and other types of cancer. In some aspects of the invention, the BRCA1 gene is mutated in subjects with ovarian cancer as compared to subjects that do not have ovarian cancer. In some embodiments, the mutations in the BRCA1 gene are Ashkenazi Jewish (AJ) mutations (e.g., c. 68_69del and/or c.5266dup).

### BRCA2

BRCA2 is another exemplary marker for use in a panel of biomarkers of the invention. The BRCA2 gene is on chromosome 13 and is 91,193 bp (NCBI Accession No. NG_012772.3). The BRCA2 protein is 3418 amino acids (NCBI Accession No. NP_000050.2) and is involved in repairing double-stranded breaks in DNA and normally helps to suppress cell growth. The amino acid sequence of an exemplary BRCA2 protein is set forth in FIG. 1. Antibodies to BRCA2 can be made using any method well known in the art, or can be purchased from, for example, Santa Cruz Biotechnology, Inc. (Catalog Number sc-293185) (www.scbt.com, Santa Cruz, Calif.).

Germline mutations in the BRCA2 gene are associated with a higher risk of breast, ovarian, prostate, and other types of cancer. In some aspects of the invention, the BRCA2 gene is mutated in subjects with ovarian cancer as compared to subjects that do not have ovarian cancer. In some embodiments, the mutations in the BRCA2 gene is an Ashkenazi Jewish (AJ) mutation (e.g., c.5946del).

### Biomarkers and Different Forms of a Protein

Proteins frequently exist in a sample in a plurality of different forms. These forms can result from pre- and/or post-translational modification. Pre-translational modified forms include allelic variants, splice variants and RNA editing forms. Post-translationally modified forms include forms resulting from proteolytic cleavage (e.g., cleavage of a signal sequence or fragments of a parent protein), glycosylation, phosphorylation, lipidation, oxidation, methylation, cysteinylation, sulphonation and acetylation. When detecting or measuring a protein in a sample, any or all of the forms may be measured to determine the level of biomarker or a form of interest is measured. The ability to differentiate between different forms of a protein depends upon the nature of the difference and the method used to detect or measure the protein. For example, an immunoassay using a monoclonal antibody will detect all forms of a protein containing the epitope and will not distinguish between them. However, a sandwich immunoassay that uses two antibodies directed against different epitopes on a protein will detect all forms of the protein that contain both epitopes and will not detect those forms that contain only one of the epitopes. Distinguishing different forms of an analyte or specifically detecting a particular form of an analyte is referred to as “resolving” the analyte.

Mass spectrometry is a particularly powerful methodology to resolve different forms of a protein because the different forms typically have different masses that can be resolved by mass spectrometry. Accordingly, if one form of a protein is a superior biomarker for a disease than another form of the biomarker, mass spectrometry may be able to specifically detect and measure the useful form where traditional immunoassay fails to distinguish the forms and fails to specifically detect to useful biomarker.

One useful methodology combines mass spectrometry with immunoassay. For example, a biospecific capture reagent (e.g., an antibody, aptamer, Affibody, and the like that recognizes the biomarker and other forms of it) is used to capture the biomarker of interest. In embodiments, the biospecific capture reagent is bound to a solid phase, such as a bead, a plate, a membrane or an array. After unbound materials are washed away, the captured analytes are detected and/or measured by mass spectrometry. This method will also result in the capture of protein interactors that are bound to the proteins or that are otherwise recognized by antibodies and that, themselves, can be biomarkers. Various forms of mass spectrometry are useful for detecting the protein forms, including laser desorption approaches, such as traditional MALDI or SELDI, electrospray ionization, and the like.

Thus, when reference is made herein to detecting a particular protein or to measuring the amount of a particular protein, it means detecting and measuring the protein with or without resolving various forms of protein. For example, the step of “detecting β-2 microglobulin” includes measuring β-2 microglobulin by means that do not differentiate between various forms of the protein (e.g., certain immunoassays) as well as by means that differentiate some forms from other forms or that measure a specific form of the protein.

### Detection of Biomarkers for Ovarian Cancer

The biomarkers of this invention can be detected by any suitable method. The methods described herein can be used individually or in combination for a more accurate detection of the biomarkers (e.g., biochip in combination with mass spectrometry, immunoassay in combination with mass spectrometry, and the like).

Detection paradigms that can be employed in the invention include, but are not limited to, optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

These and additional methods are described infra.

### Detection by Immunoassay

In particular embodiments, the biomarkers of the invention are measured by immunoassay. Immunoassay typically utilizes an antibody (or other agent that specifically binds the marker) to detect the presence or level of a biomarker in a sample. Antibodies can be produced by methods well known in the art, e.g., by immunizing animals with the biomarkers. Biomarkers can be isolated from samples based on their binding characteristics. Alternatively, if the amino acid sequence of a polypeptide biomarker is known, the polypeptide can be synthesized and used to generate antibodies by methods well known in the art.

This invention contemplates traditional immunoassays including, for example, Western blot, sandwich immunoassays including ELISA and other enzyme immunoassays, fluorescence-based immunoassays, chemiluminescence. Nephelometry is an assay done in liquid phase, in which antibodies are in solution. Binding of the antigen to the antibody results in changes in absorbance, which is measured. Other forms of immunoassay include magnetic immunoassay, radioimmunoassay, and real-time immunoquantitative PCR (iqPCR).

Immunoassays can be carried out on solid substrates (e.g., chips, beads, microfluidic platforms, membranes) or on any other forms that supports binding of the antibody to the marker and subsequent detection. A single marker may be detected at a time or a multiplex format may be used. Multiplex immunoanalysis may involve planar microarrays (protein chips) and bead-based microarrays (suspension arrays).

In a SELDI-based immunoassay, a biospecific capture reagent for the biomarker is attached to the surface of an MS probe, such as a pre-activated ProteinChip array. The biomarker is then specifically captured on the biochip through this reagent, and the captured biomarker is detected by mass spectrometry.

### Detection by Biochip

In aspects of the invention, a sample is analyzed by means of a biochip (also known as a microarray). The polypeptides and nucleic acid molecules of the invention are useful as hybridizable array elements in a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there.

The array elements are organized in an ordered fashion such that each element is present at a specified location on the substrate. Useful substrate materials include membranes, composed of paper, nylon or other materials, filters, chips, glass slides, and other solid supports. The ordered arrangement of the array elements allows hybridization patterns and intensities to be interpreted as expression levels of particular genes or proteins. Methods for making nucleic acid microarrays are known to the skilled artisan and are described, for example, in U.S. Pat. No. 5,837,832, Lockhart, et al. (Nat. Biotech. 14:1675-1680, 1996), and Schena, et al. (Proc. Natl. Acad. Sci. 93:10614-10619, 1996), herein incorporated by reference. Methods for making polypeptide microarrays are described, for example, by Ge (Nucleic Acids Res. 28: e3. i-e3. vii, 2000), MacBeath et al., (Science 289:1760-1763, 2000), Zhu et al. (Nature Genet. 26:283-289), and in U.S. Pat. No. 6,436,665, hereby incorporated by reference.

### Detection by Protein Biochip

In aspects of the invention, a sample is analyzed by means of a protein biochip (also known as a protein microarray). Such biochips are useful in high-throughput low-cost screens to identify alterations in the expression or post-translation modification of a polypeptide of the invention, or a fragment thereof. In embodiments, a protein biochip of the invention binds a biomarker present in a subject sample and detects an alteration in the level of the biomarker. Typically, a protein biochip features a protein, or fragment thereof, bound to a solid support. Suitable solid supports include membranes (e.g., membranes composed of nitrocellulose, paper, or other material), polymer-based films (e.g., polystyrene), beads, or glass slides. For some applications, proteins (e.g., antibodies that bind a marker of the invention) are spotted on a substrate using any convenient method known to the skilled artisan (e.g., by hand or by inkjet printer).

In some embodiments, the protein biochip is hybridized with a detectable probe. Such probes can be polypeptide, nucleic acid molecules, antibodies, or small molecules. For some applications, polypeptide and nucleic acid molecule probes are derived from a biological sample taken from a patient, such as a bodily fluid (such as blood, blood serum, plasma, saliva, urine, ascites, cyst fluid, and the like); a homogenized tissue sample (e.g., a tissue sample obtained by biopsy or liquid biopsy); or a cell isolated from a patient sample. Probes can also include antibodies, candidate peptides, nucleic acids, or small molecule compounds derived from a peptide, nucleic acid, or chemical library. Hybridization conditions (e.g., temperature, pH, protein concentration, and ionic strength) are optimized to promote specific interactions. Such conditions are known to the skilled artisan and are described, for example, in Harlow, E. and Lane, D., Using Antibodies: A Laboratory Manual. 1998, New York: Cold Spring Harbor Laboratories. After removal of non-specific probes, specifically bound probes are detected, for example, by fluorescence, enzyme activity (e.g., an enzyme-linked calorimetric assay), direct immunoassay, radiometric assay, or any other suitable detectable method known to the skilled artisan.

Many protein biochips are described in the art. These include, for example, protein biochips produced by Ciphergen Biosystems, Inc. (Fremont, Calif.), Zyomyx (Hayward, Calif.), Packard BioScience Company (Meriden, Conn.), Phylos (Lexington, Mass.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047; 6,537,749; 6,329,209; and 5,242,828; PCT International Publication Nos. WO 00/56934; WO 03/048768; and WO 99/51773.

### Detection by Nucleic Acid Biochip

In aspects of the invention, a sample is analyzed by means of a nucleic acid biochip (also known as a nucleic acid microarray). To produce a nucleic acid biochip, oligonucleotides may be synthesized or bound to the surface of a substrate using a chemical coupling procedure and an ink jet application apparatus, as described in PCT application WO95/251116 (Baldeschweiler et al.). Alternatively, a gridded array may be used to arrange and link cDNA fragments or oligonucleotides to the surface of a substrate using a vacuum system, thermal, UV, mechanical or chemical bonding procedure.

A nucleic acid molecule (e.g. RNA or DNA) derived from a biological sample may be used to produce a hybridization probe as described herein. The biological samples are generally derived from a patient, e.g., as a bodily fluid (such as blood, blood serum, plasma, saliva, urine, ascites, cyst fluid, and the like); a homogenized tissue sample (e.g., a tissue sample obtained by biopsy or liquid biopsy); or a cell isolated from a patient sample. For some applications, cultured cells or other tissue preparations may be used. The mRNA is isolated according to standard methods, and cDNA is produced and used as a template to make complementary RNA suitable for hybridization. Such methods are well known in the art. The RNA is amplified in the presence of fluorescent nucleotides, and the labeled probes are then incubated with the microarray to allow the probe sequence to hybridize to complementary oligonucleotides bound to the biochip.

Incubation conditions are adjusted such that hybridization occurs with precise complementary matches or with various degrees of less complementarity depending on the degree of stringency employed. For example, stringent salt concentration will ordinarily be less than about 750 mM NaCl and 75 mM trisodium citrate, less than about 500 mM NaCl and 50 mM trisodium citrate, or less than about 250 mM NaCl and 25 mM trisodium citrate. Low stringency hybridization can be obtained in the absence of organic solvent, e.g., formamide, while high stringency hybridization can be obtained in the presence of at least about 35% formamide, and most preferably at least about 50% formamide. Stringent temperature conditions will ordinarily include temperatures of at least about 30° C., of at least about 37° C., or of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred embodiment, hybridization will occur at 30° C. in 750 mM NaCl, 75 mM trisodium citrate, and 1% SDS. In embodiments, hybridization will occur at 37° C. in 500 mM NaCl, 50 mM trisodium citrate, 1% SDS, 35% formamide, and 100 μg/ml denatured salmon sperm DNA (ssDNA). In other embodiments, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

The removal of nonhybridized probes may be accomplished, for example, by washing. The washing steps that follow hybridization can also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., of at least about 42° C., or of at least about 68° C. In embodiments, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In other embodiments, wash steps will occur at 68 C in 15 mM NaCl, 1.5 mM trisodium citrate, and 0.1% SDS. Additional variations on these conditions will be readily apparent to those skilled in the art.

Detection system for measuring the absence, presence, and amount of hybridization for all of the distinct nucleic acid sequences are well known in the art. For example, simultaneous detection is described in Heller et al., Proc. Natl. Acad. Sci. 94:2150-2155, 1997. In embodiments, a scanner is used to determine the levels and patterns of fluorescence.

### Detection by Mass Spectrometry

In aspects of the invention, the biomarkers of this invention are detected by mass spectrometry (MS). Mass spectrometry is a well-known tool for analyzing chemical compounds that employs a mass spectrometer to detect gas phase ions. Mass spectrometers are well known in the art and include, but are not limited to, time-of-flight, magnetic sector, quadrupole filter, ion trap, ion cyclotron resonance, electrostatic sector analyzer and hybrids of these. The method may be performed in an automated (Villanueva, et al., Nature Protocols (2006) 1(2):880-891) or semi-automated format. This can be accomplished, for example with the mass spectrometer operably linked to a liquid chromatography device (LC-MS/MS or LC-MS) or gas chromatography device (GC-MS or GC-MS/MS). Methods for performing mass spectrometry are well known and have been disclosed, for example, in US Patent Application Publication Nos: 20050023454; 20050035286; U.S. Pat. No. 5,800,979 and the references disclosed therein.

### Laser Desorption/Ionization

In embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. The analysis of proteins by LDI can take the form of MALDI or of SELDI. The analysis of proteins by LDI can take the form of MALDI or of SELDI.

Laser desorption/ionization in a single time of flight instrument typically is performed in linear extraction mode. Tandem mass spectrometers can employ orthogonal extraction modes.

### Matrix-Assisted Laser Desorption/Ionization (MALDI) and Electrospray Ionization (ESI)

In embodiments, the mass spectrometric technique for use in the invention is matrix-assisted laser desorption/ionization (MALDI) or electrospray ionization (ESI). In related embodiments, the procedure is MALDI with time of flight (TOF) analysis, known as MALDI-TOF MS. This involves forming a matrix on a membrane with an agent that absorbs the incident light strongly at the particular wavelength employed. The sample is excited by UV or IR laser light into the vapor phase in the MALDI mass spectrometer. Ions are generated by the vaporization and form an ion plume. The ions are accelerated in an electric field and separated according to their time of travel along a given distance, giving a mass/charge (m/z) reading which is very accurate and sensitive. MALDI spectrometers are well known in the art and are commercially available from, for example, PerSeptive Biosystems, Inc. (Framingham, Mass., USA).

Magnetic-based serum processing can be combined with traditional MALDI-TOF. Through this approach, improved peptide capture is achieved prior to matrix mixture and deposition of the sample on MALDI target plates. Accordingly, in embodiments, methods of peptide capture are enhanced through the use of derivatized magnetic bead based sample processing.

MALDI-TOF MS allows scanning of the fragments of many proteins at once. Thus, many proteins can be run simultaneously on a polyacrylamide gel, subjected to a method of the invention to produce an array of spots on a collecting membrane, and the array may be analyzed. Subsequently, automated output of the results is provided by using a server (e.g., ExPASy) to generate the data in a form suitable for computers.

Other techniques for improving the mass accuracy and sensitivity of the MALDI-TOF MS can be used to analyze the fragments of protein obtained on a collection membrane. These include, but are not limited to, the use of delayed ion extraction, energy reflectors, ion-trap modules, and the like. In addition, post source decay and MS-MS analysis are useful to provide further structural analysis. With ESI, the sample is in the liquid phase and the analysis can be by ion-trap, TOF, single quadrupole, multi-quadrupole mass spectrometers, and the like. The use of such devices (other than a single quadrupole) allows MS-MS or MSⁿ analysis to be performed. Tandem mass spectrometry allows multiple reactions to be monitored at the same time.

Capillary infusion may be employed to introduce the marker to a desired mass spectrometer implementation, for instance, because it can efficiently introduce small quantities of a sample into a mass spectrometer without destroying the vacuum. Capillary columns are routinely used to interface the ionization source of a mass spectrometer with other separation techniques including, but not limited to, gas chromatography (GC) and liquid chromatography (LC). GC and LC can serve to separate a solution into its different components prior to mass analysis. Such techniques are readily combined with mass spectrometry. One variation of the technique is the coupling of high performance liquid chromatography (HPLC) to a mass spectrometer for integrated sample separation/and mass spectrometer analysis.

Quadrupole mass analyzers may also be employed as needed to practice the invention. Fourier-transform ion cyclotron resonance (FTMS) can also be used for some invention embodiments. It offers high resolution and the ability of tandem mass spectrometry experiments. FTMS is based on the principle of a charged particle orbiting in the presence of a magnetic field. Coupled to ESI and MALDI, FTMS offers high accuracy with errors as low as 0.001%.

### Surface-Enhanced Laser Desorption/Ionization (SELDI)

In embodiments, the mass spectrometric technique for use in the invention is “Surface Enhanced Laser Desorption and Ionization” or “SELDI,” as described, for example, in U.S. Pat. Nos. 5,719,060 and 6,225,047, both to Hutchens and Yip. This refers to a method of desorption/ionization gas phase ion spectrometry (e.g., mass spectrometry) in which an analyte (here, one or more of the biomarkers) is captured on the surface of a SELDI mass spectrometry probe.

SELDI has also been called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047. A “bioselective adsorbent” refers to an adsorbent that binds to an analyte with an affinity of at least 10⁻⁸ M.

Protein biochips produced by Ciphergen comprise surfaces having chromatographic or biospecific adsorbents attached thereto at addressable locations. Ciphergen's ProteinChip® arrays include NP20 (hydrophilic); H4 and H50 (hydrophobic); SAX-2, Q-10 and (anion exchange); WCX-2 and CM-10 (cation exchange); IMAC-3, IMAC-30 and IMAC-50 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities (IMAC 3 and IMAC 30) or O-methacryloyl-N,N-bis-carboxymethyl tyrosine functionalities (IMAC 50) that adsorb transition metal ions, such as copper, nickel, zinc, and gallium, by chelation. Preactivated ProteinChip arrays have acyl-imidizole or epoxide functional groups that can react with groups on proteins for covalent binding.

Such biochips are further described in: U.S. Pat. No. 6,579,719 (Hutchens and Yip, “Retentate Chromatography,” Jun. 17, 2003); U.S. Pat. No. 6,897,072 (Rich et al., “Probes for a Gas Phase Ion Spectrometer,” May 24, 2005); U.S. Pat. No. 6,555,813 (Beecher et al., “Sample Holder with Hydrophobic Coating for Gas Phase Mass Spectrometer,” Apr. 29, 2003); U.S. Patent Publication No. U.S. 2003-0032043 A1 (Pohl and Papanu, “Latex Based Adsorbent Chip,” Jul. 16, 2002); and PCT International Publication No. WO 03/040700 (Um et al., “Hydrophobic Surface Chip,” May 15, 2003); U.S. Patent Application Publication No. US 2003/-0218130 A1 (Boschetti et al., “Biochips With Surfaces Coated With Polysaccharide-Based Hydrogels,” Apr. 14, 2003) and U.S. Pat. No. 7,045,366 (Huang et al., “Photocrosslinked Hydrogel Blend Surface Coatings” May 16, 2006).

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature. Unless the probe has both SEAC and SEND properties (as described herein), an energy absorbing molecule then is applied to the substrate with the bound biomarkers.

In yet another method, one can capture the biomarkers with a solid-phase bound immuno-adsorbent that has antibodies that bind the biomarkers. After washing the adsorbent to remove unbound material, the biomarkers are eluted from the solid phase and detected by applying to a SELDI biochip that binds the biomarkers and analyzing by SELDI.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

### Sequencing for Identifying Mutations

In some embodiments, a nucleic acid molecule (e.g. RNA or DNA) derived from a biological sample (e.g., detected by a nucleic acid biochip) may be sequenced to identify a particular mutation, such as a germline mutation (e.g., BRCA1/2) or a somatic mutation, associated with ovarian cancer. In some embodiments, next-generation sequencing or Sanger sequencing may be used. Sequencing methods are well known to those skilled in the art and one of ordinary skill can readily select and use the appropriate sequencing method to analyze a particular mutation.

### METHODS OF THE INVENTION

Panels comprising biomarkers of the invention are used to characterize a pelvic mass in a subject to determine whether the subject should be seen by a general surgeon or should be evaluated and/or treated by a gynecologic oncologist. In other embodiments, a panel of the invention is used to diagnose or stage an ovarian cancer by determining the molecular profile of the cancer. In certain embodiments, panels of the invention are used to select a course of treatment for a subject. The phrase “ovarian cancer status” includes any distinguishable manifestation of the disease, including non-disease. For example, ovarian cancer status includes, without limitation, the presence or absence of disease (e.g., ovarian cancer v. non-ovarian cancer), the risk of developing disease, the stage of the disease, the progression of disease (e.g., progress of disease or remission of disease over time), prognosis, the effectiveness or response to treatment of disease, and the determination of whether a pelvic mass is malignant of benign, symptomatic or asymptomatic. Based on this status, further procedures may be indicated, including additional diagnostic tests or therapeutic procedures or regimens. In aspects of the invention, the biomarkers of the invention can be used in diagnostic tests to identify early stage ovarian cancer in a subject.

In some embodiments, the methods of the invention pre-operatively assess a subject as having a high, intermediate or low risk of ovarian cancer. This method involves measuring or characterizing a panel of biomarkers in a subject. The treatment of disease (e.g., surgery) is determined based on this characterization.

In some embodiments, the panel of biomarkers include, but are not limited to, Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH). In some embodiments, multiple panels of biomarkers are measured or characterized in a subject. In one embodiment, a first panel of markers including, but is not limited to, TT, ApoA1, β2M, Tfr, CA125 HE4 and FSH is characterized and a second panel of markers including, but not limited to, CA125, β2M, Tfr, TT and ApoA1 is characterized. In one embodiment, a first panel of markers including, but is not limited to, TT, ApoA1, β2M, Tfr, CA125 HE4 and FSH is characterized and a second panel of markers including, but not limited to, FSH, CA125, HE4, Tfr, and ApoA1 is characterized. In another embodiment, a first panel of markers including, but is not limited to, TT, ApoA1, β2M, Tfr, CA125 HE4 and FSH is characterized, a second panel of markers including, but is not limited to, FSH, CA125, HE4, Tfr, and ApoA1 is characterized, and a third panel of markers including, but is not limited to, CA125, β2M, Tfr, TT and ApoA1 is characterized.

In some embodiments, the characterization of a panel of biomarkers in a biological sample from a subject determines a score that identifies that subject as having a low, intermediate or high risk of developing or having ovarian cancer. In some embodiments, the characterization of a first panel of markers determines a first score. In some embodiments, a subject identified by the first score with an intermediate risk of developing or having ovarian cancer is selected for further characterization with one or more panels of biomarkers. In some embodiments, the characterization of a second panel of markers determines a second score. In some embodiments, the characterization of a third panel of markers determines a third score. In some embodiments, the first score identifies a subject as having a high, intermediate, or low risk of developing ovarian cancer. In some embodiments, the second score identifies a subject as having a high or low risk of developing ovarian cancer. In some embodiments, the second score identifies a subject as having a high, intermediate, or low risk of developing ovarian cancer. In some embodiments, the third score identifies the subject as having a low or high cancer risk.

In some embodiments, the first score ranges from 0 to 20. In some embodiments, a first score less than or equal to 5 identifies the subject as having a low cancer risk. In some embodiments, where the subject has a pre-menopausal status, a first score greater than 5 and less than 10 identifies the subject as having an intermediate cancer risk. In some embodiments, where the subject has a post-menopausal status, a first score greater than 5 and less than 14 identifies the subject as having an intermediate cancer risk. In some embodiments, a first score greater than 5 identifies the subject as having a high cancer risk. In some embodiments, where the subject has a pre-menopausal status, a first score greater than or equal to 10 identifies the subject as having a high cancer risk. In some embodiments, a first score greater than 5 identifies the subject as having a high cancer risk. In some embodiments, where the subject has a post-menopausal status, a first score greater than or equal to 14 identifies the subject as having a high cancer risk.

In some embodiments, the second score ranges from 0 to 20. In some embodiments, a second score less than or equal to 5 identifies the subject as having a low cancer risk. In some embodiments, where the subject has a pre-menopausal status, a second score less than or equal to 5 identifies the subject as having a low cancer risk. In some embodiments, where the subject has a pre-menopausal status, a second score greater than 5 and less than 10 identifies the subject as having an intermediate cancer risk. In some embodiments, where the subject has a post-menopausal status, a second score greater than 5 and less than 14 identifies the subject as having an intermediate cancer risk. In some embodiments, where the subject has a pre-menopausal status, a second score greater than or equal to 10 identifies the subject as having a high cancer risk. In some embodiments, where the subject has a post-menopausal status, a second score greater than or equal to 14 identifies the subject as having a high cancer risk. In some embodiments, where the subject has a post-menopausal status, a second score less than 4.4 identifies the subject as having a low cancer risk. In some embodiments, where the subject has a pre-menopausal status, a second score greater than 5 and less than 7 identifies the subject as having an intermediate cancer risk. In some embodiments, where the subject has a post-menopausal status, a second score greater than 4.4 and less than 6 identifies the subject as having an intermediate cancer risk. In some embodiments, a second score greater than 5 identifies the subject as having a high cancer risk. In some embodiments, where the subject has a pre-menopausal status, a second score greater than or equal to 7 identifies the subject as having a high cancer risk. In some embodiments, where the subject has a post-menopausal status, a second score greater than or equal to 6 identifies the subject as having a high cancer risk.

In some embodiments, the third score ranges from 0 to 20. In some embodiments, a third score less than or equal to 5 identifies the subject as having a low cancer risk. In some embodiments, a third score greater than 5 identifies the subject as having a high cancer risk.

In some embodiments, a biological sample from a subject is further characterized by detecting whether the subject has one or more mutations in one or more germline and/or somatic markers. In some embodiments, the germline and/or somatic markers are associated with breast and/or ovarian cancer. In some embodiments, the presence of one or more mutations in one or more breast and/or ovarian cancer markers identifies a subject as in need of therapeutic intervention having a higher [increased] cancer risk relative to a subject that does not have one of these markers. In some embodiments, aberrant methylation of one or more breast and/or ovarian cancer markers identifies a subject as in need of therapeutic intervention having a higher [increased] cancer risk relative to a subject that does not have aberrant methylation of one of these markers. In some embodiments, the aberrant methylation of one or more breast and/or ovarian cancer markers is hypermethylation. In some embodiments, the aberrant methylation of one or more of the above breast and/or ovarian cancer markers is hypomethylation. In some embodiments, a subject identified as having a low, intermediate or high risk of ovarian cancer and as having one or more mutations in one or more germline and/or somatic markers is further identified as in need of therapeutic intervention (e.g., surgery).

In some embodiments, the one or more germline and/or somatic markers associated with breast and/or ovarian cancer include, but are not limited to, Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), and Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P16INK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1).

In some embodiments, the germline markers are BRCA1 and/or BRCA 2. In some embodiments, the BRCA1 mutation is 68_69del and/or c.5266dup. In some embodiments, the BRCA2 mutation is c.5946del.

In some embodiments, methods for pre-operatively assessing a subject as having a high, intermediate or low risk of ovarian cancer further includes characterizing one or more clinical biomarkers of ovarian cancer risk in the subject, wherein the one or more clinical biomarkers are selected from group consisting of age, pre-menopausal status, post-menopausal status, ethnicity, pathology, adnexal mass diagnosis, family history, physical examination, imaging results, and/or history of smoking, wherein the one or more clinical biomarkers further identifies the subject as having a low or high cancer risk. In some embodiments, the subject is diagnosed with an adnexal mass. In some embodiments, the subject is diagnosed with an asymptomatic adnexal mass. In some embodiments, the subject is diagnosed with a symptomatic adnexal mass. In some embodiments, the subject is pre-menopausal. In some embodiments, the subject is post-menopausal.

The method includes a diagnostic measurement (e.g., screening assay or detection assay) in a biological sample obtained from the subject suffering from or susceptible to ovarian cancer. In some embodiments, the diagnostic measurement characterizes markers in a biological sample. In some embodiments, the biological sample is serum. In some embodiments, one or more markers are characterized by detecting cell-free tumor DNA (cftDNA). In some embodiments, a panel of markers are bound to a separate capture reagent. In some embodiments, the capture reagents are attached to a solid support. In some embodiments, the solid support is a plate, chip, beads, microfluidic platform, membrane, planar microarray, or suspension array. In some embodiments, the capture reagent is an antibody, aptamer, Affibody, hybridization probe and/or fragments thereof each capture reagent specifically binds to one of the markers. In some embodiments, the markers are characterized by immunoassay, sequencing and/or nucleic acid microarray. In some embodiments, the sequencing is next-generation sequencing (NGS) or Sanger sequencing. In some embodiments, the immunoassay comprises affinity capture assay, immunometric assay, heterogeneous chemiluminscence immunometric assay, homogeneous chemiluminscence immunometric assay, ELISA, western blotting, radioimmunoassay, magnetic immunoassay, real-time immunoquantitative PCR (iqPCR) and SERS label free assay.

The correlation of test results with ovarian cancer involves applying a classification algorithm of some kind to the results to generate the status. The classification algorithm may be as simple as determining whether or not the amounts of the markers or a combination of the markers listed in Table 1 are above or below a particular cut-off number. When multiple biomarkers are used, the classification algorithm may be a linear regression formula. Alternatively, the classification algorithm may be the product of any of a number of learning algorithms described herein.

In the case of complex classification algorithms, it may be necessary to perform the algorithm on the data, thereby determining the classification, using a computer, e.g., a programmable digital computer. In either case, one can then record the status on tangible medium, for example, in computer-readable format such as a memory drive or disk or simply printed on paper. The result also could be reported on a computer screen.

### Biomarkers of the Invention

Individual biomarkers are useful diagnostic biomarkers. In addition, as described in the examples, it has been found that a specific combination of biomarkers provides greater predictive value of a particular status than any single biomarker alone, or any other combination of previously identified biomarkers. Specifically, the detection of a plurality of biomarkers in a sample can increase the sensitivity, accuracy and specificity of the test.

Each biomarkers described herein can be differentially present in ovarian cancer, and, therefore, each is individually useful in aiding in the determination of ovarian cancer status. The method involves, first, measuring the selected biomarker in a subject, sample using any method well known in the art, including but not limited to the methods described herein, e.g. capture on a SELDI biochip followed by detection by mass spectrometry and, second, comparing the measurement with a diagnostic amount or cut-off that distinguishes a positive ovarian cancer status from a negative ovarian cancer status. The diagnostic amount represents a measured amount of a biomarker above which or below which a subject is classified as having a particular ovarian cancer status. For example, if the biomarker is up-regulated compared to normal during ovarian cancer, then a measured amount above the diagnostic cutoff provides a diagnosis of ovarian cancer. Alternatively, if the biomarker is down-regulated during ovarian cancer, then a measured amount below the diagnostic cutoff provides a diagnosis of ovarian cancer. As is well understood in the art, by adjusting the particular diagnostic cut-off used in an assay, one can increase sensitivity or specificity of the diagnostic assay depending on the preference of the diagnostician. The particular diagnostic cut-off can be determined, for example, by measuring the amount of the biomarker in a statistically significant number of samples from subjects with the different ovarian cancer statuses, as was done here, and drawing the cut-off to suit the diagnostician's desired levels of specificity and sensitivity.

The biomarkers of this invention (used alone or in combination) show a statistical difference in different ovarian cancer statuses of at least p≤0.05, p≤10⁻², p≤10⁻³, p≤10⁻⁴, or p≤10⁻⁵. Diagnostic tests that use these biomarkers alone or in combination show a sensitivity and specificity of at least 75%, at least 80%, at least 85%, at least 90%, at least 95%, at least 98%, or about 100%.

### Determining Course (Progression/Remission) of Disease

In one embodiment, this invention provides methods for monitoring or determining the course of disease in a subject. Disease course refers to changes in disease status over time, including disease progression (worsening) and disease regression (improvement). Over time, the amounts or relative amounts (e.g., the pattern) of the biomarkers change. Accordingly, this method involves measuring or characterizing a panel of biomarkers in a biological sample from a subject during at least two different time points, e.g., a first time and a second time, and comparing the change in amounts, if any. The course of disease (e.g., during treatment) is determined based on these comparisons.

In some embodiments, the panel of biomarkers include, but are not limited to, Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH). In some embodiments, multiple panels of biomarkers are measured or characterized in a biological sample from a subject during at least two different time points. In one embodiment, a first panel of markers including, but is not limited to, TT, ApoA1, β2M, Tfr, CA125 HE4 and FSH is characterized and a second panel of markers including CA125, β2M, Tfr, TT and ApoA1 is characterized. In one embodiment, a first panel of markers including, but is not limited to, TT, ApoA1, β2M, Tfr, CA125 HE4 and FSH is characterized and a second panel of markers including FSH, CA125, HE4, Tfr, and ApoA1 is characterized. In another embodiment, a first panel of markers including, but is not limited to, TT, ApoA1, β2M, Tfr, CA125 HE4 and FSH is characterized, a second panel of markers including, but is not limited to, FSH, CA125, HE4, Tfr, and ApoA1 is characterized, and a third panel of markers including, but is not limited to, CA125, β2M, Tfr, TT and ApoA1 is characterized.

In some embodiments, the characterization of a panel of biomarkers determines a score that identifies a subject as having a low, intermediate or high risk of developing ovarian cancer, which can be compared at different time points to monitor the course of disease. In some embodiments, the characterization of a first panel of markers determines a first score. In some embodiments, a subject identified from the first score with a low or intermediate risk of ovarian cancer is selected for further characterization using one or more additional panels of biomarkers. In some embodiments, the characterization of a second panel of markers determines a second score. In some embodiments, the characterization of a third panel of markers determines a third score. In some embodiments, the first score identifies a subject as having a high, intermediate, or low risk of developing ovarian cancer. In some embodiments, the second score identifies a subject as having a high or low risk of developing ovarian cancer. In some embodiments, the second score identifies a subject as having a high, intermediate, or low risk of developing ovarian cancer. In some embodiments, the third score identifies the subject as having a low or high cancer risk.

In some embodiments, the first score ranges from 0 to 20. In some embodiments, a first score less than or equal to 5 identifies the subject as having a low cancer risk. In some embodiments, where the subject has a pre-menopausal status, a first score greater than 5 and less than 10 identifies the subject as having an intermediate cancer risk. In some embodiments, where the subject has a post-menopausal status, a first score greater than 5 and less than 14 identifies the subject as having an intermediate cancer risk. In some embodiments, a first score greater than 5 identifies the subject as having a high cancer risk. In some embodiments, where the subject has a pre-menopausal status, a first score greater than or equal to 10 identifies the subject as having a high cancer risk. In some embodiments, a first score greater than 5 identifies the subject as having a high cancer risk. In some embodiments, where the subject has a post-menopausal status, a first score greater than or equal to 14 identifies the subject as having a high cancer risk.

In some embodiments, the second score ranges from 0 to 20. In some embodiments, a second score less than or equal to 5 identifies the subject as having a low cancer risk. In some embodiments, where the subject has a pre-menopausal status, a second score less than or equal to 5 identifies the subject as having a low cancer risk. In some embodiments, where the subject has a pre-menopausal status, a second score greater than 5 and less than 10 identifies the subject as having an intermediate cancer risk. In some embodiments, where the subject has a post-menopausal status, a second score greater than 5 and less than 14 identifies the subject as having an intermediate cancer risk. In some embodiments, where the subject has a pre-menopausal status, a second score greater than or equal to 10 identifies the subject as having a high cancer risk. In some embodiments, where the subject has a post-menopausal status, a second score greater than or equal to 14 identifies the subject as having a high cancer risk. In some embodiments, where the subject has a post-menopausal status, a second score less than 4.4 identifies the subject as having a low cancer risk. In some embodiments, where the subject has a pre-menopausal status, a second score greater than 5 and less than 7 identifies the subject as having an intermediate cancer risk. In some embodiments, where the subject has a post-menopausal status, a second score greater than 4.4 and less than 6 identifies the subject as having an intermediate cancer risk. In some embodiments, a second score greater than 5 identifies the subject as having a high cancer risk. In some embodiments, where the subject has a pre-menopausal status, a second score greater than or equal to 7 identifies the subject as having a high cancer risk. In some embodiments, where the subject has a post-menopausal status, a second score greater than or equal to 6 identifies the subject as having a high cancer risk.

In some embodiments, the third score ranges from 0 to 20. In some embodiments, a third score less than or equal to 5 identifies the subject as having a low cancer risk. In some embodiments, a third score greater than 5 identifies the subject as having a high cancer risk.

In some embodiments, subjects with a low or intermediate risk of developing ovarian cancer are monitored for disease progression (i.e., development of high risk status). In some embodiments, a subject with one or more mutations in one or more germline and/or somatic markers having a low or intermediate risk of developing ovarian cancer is monitored for disease progression (i.e., development of high risk status). In some embodiments, a subject with aberrant methylation in one or more germline and/or somatic markers having a low or intermediate risk of developing ovarian cancer is monitored for disease progression (i.e., development of high risk status). In some embodiments, the aberrant methylation is hypermethylation. In some embodiments, the aberrant methylation is hypomethylation.

In some embodiments, the one or more germline and/or somatic markers include, but are not limited to, Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS) BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), and Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXADI1), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P16INK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1).

In some embodiments, the germline markers are BRCA1 and/or BRCA 2. In some embodiments, the BRCA1 mutation is 68_69del and/or c.5266dup. In some embodiments, the BRCA2 mutation is c.5946del.

In some embodiments, methods for monitoring or determining the course of disease in a subject further characterizes one or more clinical biomarkers of ovarian cancer risk in the subject, wherein the one or more clinical biomarkers are selected from group consisting of age, pre-menopausal status, post-menopausal status, ethnicity, pathology, adnexal mass diagnosis, family history, physical examination, imaging results, and/or history of smoking, wherein the one or more clinical biomarkers further identifies the subject as having a low or high cancer risk. In some embodiments, a subject diagnosed with an adnexal mass having a low or intermediate risk of developing ovarian cancer is monitored for disease progression (i.e., high risk status). In some embodiments, the subject is diagnosed with an asymptomatic adnexal mass. In some embodiments, the subject is diagnosed with a symptomatic adnexal mass. In some embodiments, the subject is pre-menopausal. In some embodiments, the subject is post-menopausal.

The method includes a diagnostic measurement (e.g., screening assay or detection assay) in a biological sample obtained from the subject suffering from or susceptible to ovarian cancer. In some embodiments, the diagnostic measurement characterizes markers in a biological sample. In some embodiments, the biological sample is serum. In some embodiments, one or more markers are characterized by detecting cell-free tumor DNA (cftDNA). In some embodiments, a panel of markers are bound to a separate capture reagent. In some embodiments, the capture reagents are attached to a solid support. In some embodiments, the solid support is a plate, chip, beads, microfluidic platform, membrane, planar microarray, or suspension array. In some embodiments, the capture reagent is an antibody, aptamer, Affibody, hybridization probe and/or fragments thereof each capture reagent specifically binds to one of the markers. In some embodiments, the markers are characterized by immunoassay, sequencing and/or nucleic acid microarray. In some embodiments, the sequencing is next-generation sequencing (NGS) or Sanger sequencing. In some embodiments, the immunoassay comprises affinity capture assay, immunometric assay, heterogeneous chemiluminscence immunometric assay, homogeneous chemiluminscence immunometric assay, ELISA, western blotting, radioimmunoassay, magnetic immunoassay, real-time immunoquantitative PCR (iqPCR) and SERS label free assay.

The diagnostic measurement in the method can be compared to samples from healthy, normal controls; in a pre-disease sample of the subject; or in other afflicted/diseased patients to establish the treated subject's disease status. For monitoring, a second diagnostic measurement may be obtained from the subject at a time point later than the determination of the first diagnostic measurement, and the two measurements can be compared to monitor the course of disease or the efficacy of the therapy/treatment. In certain embodiments, a pre-treatment measurement in the subject (e.g., in a sample or biopsy obtained from the subject or CT scan) is determined prior to beginning treatment as described; this measurement can then be compared to a measurement in the subject after the treatment commences and/or during the course of treatment to determine the efficacy of (monitor the efficacy of) the disease treatment. In some embodiments, efficacy of the disease treatment can be performed with antibody marker analysis and/or interferon-gamma (IFN-γ) ELISPOT assays.

### Reporting the Status

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

In a preferred embodiment of the invention, a diagnosis based on the differential presence or absence in a test subject of the biomarkers or a combination of the biomarkers of Table 1 is communicated to the subject as soon as possible after the diagnosis is obtained. The diagnosis may be communicated to the subject by the subject's treating physician. Alternatively, the diagnosis may be sent to a test subject by email or communicated to the subject by phone. A computer may be used to communicate the diagnosis by email or phone. In certain embodiments, the message containing results of a diagnostic test may be generated and delivered automatically to the subject using a combination of computer hardware and software which will be familiar to artisans skilled in telecommunications. One example of a healthcare-oriented communications system is described in U.S. Pat. No. 6,283,761; however, the present invention is not limited to methods which utilize this particular communications system. In certain embodiments of the methods of the invention, all or some of the method steps, including the assaying of samples, diagnosing of diseases, and communicating of assay results or diagnoses, may be carried out in diverse (e.g., foreign) jurisdictions.

### Subject Management

In certain embodiments, the methods of the invention involve managing subject treatment based on the status. Such management includes referral, for example, to a gynecologic oncologist, or other actions of the physician or clinician subsequent to determining ovarian cancer status. For example, if a physician makes a diagnosis of ovarian cancer, then a certain regime of treatment, such as prescription or administration of therapeutic agent might follow. Alternatively, a diagnosis of non-ovarian cancer or non-ovarian cancer might be followed with further testing to determine a specific disease that might the patient might be suffering from. Also, if the diagnostic test gives an inconclusive result on ovarian cancer status, further tests may be called for.

In one embodiment, the diagnosis may be determining if a pelvic mass is benign or malignant. If the diagnosis is malignant, a gynecologic oncologist may be chosen to perform the surgery. In contrast, if the diagnosis is benign, a general surgeon or a gynecologist may be chosen to perform the surgery.

Additional embodiments of the invention relate to the communication of assay results or diagnoses or both to technicians, physicians or patients, for example. In certain embodiments, computers will be used to communicate assay results or diagnoses or both to interested parties, e.g., physicians and their patients. In some embodiments, the assays will be performed or the assay results analyzed in a country or jurisdiction which differs from the country or jurisdiction to which the results or diagnoses are communicated.

### Hardware and Software

The any of the methods described herein, the step of correlating the measurement of the biomarker(s) with ovarian cancer can be performed on general-purpose or specially-programmed hardware or software.

In aspects, the analysis is performed by a software classification algorithm. The analysis of analytes by any detection method well known in the art, including, but not limited to the methods described herein, will generate results that are subject to data processing. Data processing can be performed by the software classification algorithm. Such software classification algorithms are well known in the art and one of ordinary skill can readily select and use the appropriate software to analyze the results obtained from a specific detection method.

In aspects, the analysis is performed by a computer-readable medium. The computer-readable medium can be non-transitory and/or tangible. For example, the computer readable medium can be volatile memory (e.g., random access memory and the like) or non-volatile memory (e.g., read-only memory, hard disks, floppy discs, magnetic tape, optical discs, paper table, punch cards, and the like).

For example, analysis of analytes by time-of-flight mass spectrometry generates a time-of-flight spectrum. The time-of-flight spectrum ultimately analyzed typically does not represent the signal from a single pulse of ionizing energy against a sample, but rather the sum of signals from a number of pulses. This reduces noise and increases dynamic range. This time-of-flight data is then subject to data processing. Exemplary software includes, but is not limited to, Ciphergen's ProteinChip® software, in which data processing typically includes TOF-to-M/Z transformation to generate a mass spectrum, baseline subtraction to eliminate instrument offsets and high frequency noise filtering to reduce high frequency noise.

Data generated by desorption and detection of biomarkers can be analyzed with the use of a programmable digital computer. The computer program analyzes the data to indicate the number of biomarkers detected, and optionally the strength of the signal and the determined molecular mass for each biomarker detected. Data analysis can include steps of determining signal strength of a biomarker and removing data deviating from a predetermined statistical distribution. For example, the observed peaks can be normalized, by calculating the height of each peak relative to some reference. The reference can be background noise generated by the instrument and chemicals such as the energy absorbing molecule which is set at zero in the scale.

The computer can transform the resulting data into various formats for display. The standard spectrum can be displayed, but in one useful format only the peak height and mass information are retained from the spectrum view, yielding a cleaner image and enabling biomarkers with nearly identical molecular weights to be more easily seen. In another useful format, two or more spectra are compared, conveniently highlighting unique biomarkers and biomarkers that are up- or down-regulated between samples. Using any of these formats, one can readily determine whether a particular biomarker is present in a sample.

Analysis generally involves the identification of peaks in the spectrum that represent signal from an analyte. Peak selection can be done visually, but software is available, for example, as part of Ciphergen's ProteinChip® software package, that can automate the detection of peaks. This software functions by identifying signals having a signal-to-noise ratio above a selected threshold and labeling the mass of the peak at the centroid of the peak signal. In embodiments, many spectra are compared to identify identical peaks present in some selected percentage of the mass spectra. One version of this software clusters all peaks appearing in the various spectra within a defined mass range, and assigns a mass (N/Z) to all the peaks that are near the mid-point of the mass (M/Z) cluster.

In aspects, software used to analyze the data can include code that applies an algorithm to the analysis of the results (e.g., signal to determine whether the signal represents a peak in a signal that corresponds to a biomarker according to the present invention). The software also can subject the data regarding observed biomarker peaks to classification tree or ANN analysis, to determine whether a biomarker peak or combination of biomarker peaks is present that indicates the status of the particular clinical parameter under examination. Analysis of the data may be “keyed” to a variety of parameters that are obtained, either directly or indirectly, from the mass spectrometric analysis of the sample. These parameters include, but are not limited to, the presence or absence of one or more peaks, the shape of a peak or group of peaks, the height of one or more peaks, the log of the height of one or more peaks, and other arithmetic manipulations of peak height data.

### Classification Algorithms for Qualifying Ovarian Cancer Status

In some embodiments, data derived from the assays (e.g., ELISA assays) that are generated using samples such as “known samples” can then be used to “train” a classification model. A “known sample” is a sample that has been pre-classified. The data that are derived from the spectra and are used to form the classification model can be referred to as a “training data set.” Once trained, the classification model can recognize patterns in data derived from spectra generated using unknown samples. The classification model can then be used to classify the unknown samples into classes. This can be useful, for example, in predicting whether or not a particular biological sample is associated with a certain biological condition (e.g., diseased versus non-diseased).

The training data set that is used to form the classification model may comprise raw data or pre-processed data. In some embodiments, raw data can be obtained directly from time-of-flight spectra or mass spectra, and then may be optionally “pre-processed” as described above.

Classification models can be formed using any suitable statistical classification (or “learning”) method that attempts to segregate bodies of data into classes based on objective parameters present in the data. Classification methods may be either supervised or unsupervised. Examples of supervised and unsupervised classification processes are described in Jain, “Statistical Pattern Recognition: A Review”, IEEE Transactions on Pattern Analysis and Machine Intelligence, Vol. 22, No. 1, January 2000, the teachings of which are incorporated by reference.

In supervised classification, training data containing examples of known categories are presented to a learning mechanism, which learns one or more sets of relationships that define each of the known classes. New data may then be applied to the learning mechanism, which then classifies the new data using the learned relationships. Examples of supervised classification processes include linear regression processes (e.g., multiple linear regression (MLR), partial least squares (PLS) regression and principal components regression (PCR)), binary decision trees (e.g., recursive partitioning processes such as CART—classification and regression trees), artificial neural networks such as back propagation networks, discriminant analyses (e.g., Bayesian classifier or Fischer analysis), logistic classifiers, and support vector classifiers (support vector machines).

In embodiments, a supervised classification method is a recursive partitioning process. Recursive partitioning processes use recursive partitioning trees to classify spectra derived from unknown samples. Further details about recursive partitioning processes are provided in U.S. Patent Application No. 2002 0138208 A1 to Paulse et al., “Method for analyzing mass spectra.”

In other embodiments, the classification models that are created can be formed using unsupervised learning methods. Unsupervised classification attempts to learn classifications based on similarities in the training data set, without pre-classifying the spectra from which the training data set was derived. Unsupervised learning methods include cluster analyses. A cluster analysis attempts to divide the data into “clusters” or groups that ideally should have members that are very similar to each other, and very dissimilar to members of other clusters. Similarity is then measured using some distance metric, which measures the distance between data items, and clusters together data items that are closer to each other. Clustering techniques include the MacQueen's K-means algorithm and the Kohonen's Self-Organizing Map algorithm.

Learning algorithms asserted for use in classifying biological information are described, for example, in PCT International Publication No. WO 01/31580 (Barnhill et al., “Methods and devices for identifying patterns in biological systems and methods of use thereof”), U.S. Patent Application No. 2002 0193950 A1 (Gavin et al., “Method or analyzing mass spectra”), U.S. Patent Application No. 2003 0004402 A1 (Hitt et al., “Process for discriminating between biological states based on hidden patterns from biological data”), and U.S. Patent Application No. 2003 0055615 A1 (Zhang and Zhang, “Systems and methods for processing biological expression data”).

The classification models can be formed on and used on any suitable digital computer. Suitable digital computers include micro, mini, or large computers using any standard or specialized operating system, such as a Unix, Windows™ or Linux™ based operating system. The digital computer that is used may be physically separate from the mass spectrometer that is used to create the spectra of interest, or it may be coupled to the mass spectrometer.

The training data set and the classification models according to embodiments of the invention can be embodied by computer code that is executed or used by a digital computer. The computer code can be stored on any suitable computer readable media including optical or magnetic disks, sticks, tapes, etc., and can be written in any suitable computer programming language including C, C++, visual basic, etc.

The learning algorithms described above are useful both for developing classification algorithms for the biomarkers already discovered, or for finding new biomarkers for ovarian cancer. The classification algorithms, in turn, form the base for diagnostic tests by providing diagnostic values (e.g., cut-off points) for biomarkers used singly or in combination.

In some embodiments, the methods of the invention include classifying a subject's risk of having ovarian cancer. In some embodiments, the method includes receiving, by at least one processor, a signal representing a marker spectrum peak detected for each marker of a panel. In some embodiments, one or more panels are used. In some embodiments, the panel includes, but is not limited to, markers Transthyretin/prealbumin (TT), Apolipoprotein A1 (ApoA1), β2-Microglobulin (β2M), Transferrin (Tfr), Cancer Antigen 125 (CA125), HE4, and follicle stimulating hormone (FSH). In some embodiments, the panel includes, but is not limited to, one or more markers selected from Breast Cancer 1 (BRCA1), Breast Cancer 2 (BRCA2), ATM, BARD1, BRIP1, CDH1, CHEK2, EPCAM, MLH1, MSH2, MSH6, NBN, PALB2, PTEN, RAD51D, STK11, TP53, KRAS, ABRAXAS1, AKT1, APC, AXIN2, BMPR1A, BRAF, CDC25, CDKN2A, CDK4, CTNNB1, DICER1, ERBB2, ERCC6, FANCM, FANCC, MRE11, MUTYH, NF1, NTHL1, PIK3CA, PMS2, PP2R1A, PRKDC, POLD1, RAD50, RAD51C, RNF43, SDHB, SDHD, SMARCA4, XRCC2, WRN, CDC73, GALNT12, GREM1, HOXB13, MSH3, POLE, RAD51, RINT1, RSP20, SLX4, SMAD4, TTK, RASSF1A, RUNX3, TFPI2, SFRP5, OPCML, MGMT, CDH13, SULF1, HOXA9, HOXAD11, CLDN4, MAL, BORIS, ABCG2, TUBB3, MCJ, SNGG, P14ARF, P161NK4A, DAPK, P15, MINT31, RIZ1, and TMS1. In some embodiments, the panel includes, but is not limited to, CA125, β2M, Tfr, TT and ApoA1. In some embodiments, the panel includes, but is not limited to, FSH, CA125, HE4, Transferrin, and ApoA1.

In some embodiments, the method includes receiving, by at least one processor, a first panel signal representing a marker spectrum peak detected for each marker of a panel comprising markers Transthyretin/prealbumin (TT), Apolipoprotein A1 (ApoA1), β2-Microglobulin (β2M), Transferrin (Tfr), Cancer Antigen 125 (CA125), HE4, and follicle stimulating hormone (FSH) and one or more markers selected from the group consisting of Breast Cancer 1 (BRCA1), Breast Cancer 2 (BRCA2), ATM, BARD1, BRIP1, CDH1, CHEK2, EPCAM, MLH1, MSH2, MSH6, NBN, PALB2, PTEN, RAD51D, STK11, TP53, KRAS, ABRAXAS1, AKT1, APC, AXIN2, BMPR1A, BRAF, CDC25, CDKN2A, CDK4, CTNNB1, DICER1, ERBB2, ERCC6, FANCM, FANCC, MRE11, MUTYH, NF1, NTHL1, PIK3CA, PMS2, PP2R1A, PRKDC, POLD1, RAD50, RAD51C, RNF43, SDHB, SDHD, SMARCA4, XRCC2, WRN, CDC73, GALNT12, GREM1, HOXB13, MSH3, POLE, RAD51, RINT1, RSP20, SLX4, SMAD4, TTK, RASSFlA, RUNX3, TFPI2, SFRP5, OPCML, MGMT, CDH13, SULF1, HOXA9, HOXAD11, CLDN4, MAL, BORIS, ABCG2, TUBB3, MCJ, SNGG, P14ARF, P16INK4A, DAPK, P15, MINT31, RIZ1, and TMS1.

In some embodiments, the method utilizes, by the at least one processor, a first stage cancer risk classifier to predict a cancer risk classification score representative of a predicted risk of developing ovarian cancer, the cancer risk classification score being based on learned risk classification parameters and the first panel signal. In some embodiments, the method determines, by the at least one processor, a cancer risk level associated with the cancer risk classification score, the cancer risk level selected from one of at least the selection comprising low risk, intermediate risk and high risk. In some embodiments, the method generates, by the at least one processor, a cancer risk level prediction at a computing device associated with a care provider indicative of the cancer risk level of the subject.

In some embodiments, the method further includes determining, by the at least one processor, the cancer risk level as intermediate risk; utilizing, by the at least one processor, a second stage cancer risk classifier to predict an enhanced cancer risk classification score based on second stage learned risk classification parameters and second panel signal comprising a subset of the first panel signal; and determining, by the at least one processor, an enhanced cancer risk level associated with the enhanced cancer risk classification score, the enhanced cancer risk level selected from one of at least the selection comprising low risk and high risk. In some embodiments, the second panel signal represents the marker spectrum peak of markers comprising or consisting of CA125, β2M, Transferrin, Transthyretin and ApoA1. In some embodiments, the second panel signal represents the marker spectrum peak of markers comprising or consisting of FSH, CA125, HE4, Transferrin, ApoA1.

In some embodiments, the method further includes determining, by the at least one processor, the cancer risk level as intermediate risk; utilizing, by the at least one processor, a second stage cancer risk classifier and a third stage cancer risk classifier to predict an enhanced cancer risk classification score based on second stage and third stage learned risk classification parameters and second and third panel signals each comprising a different subset of the first panel signal; and determining, by the at least one processor, an enhanced cancer risk level associated with the enhanced cancer risk classification score, the enhanced cancer risk level selected from one of at least the selection comprising low risk and high risk. In some embodiments, the second panel signal represents the marker spectrum peak of markers comprising or consisting of CA125, β2M, Transferrin, Transthyretin and ApoA1 and the third panel signal represents the marker spectrum peak of markers comprising or consisting of FSH, CA125, HE4, Transferrin, ApoA1.

In some embodiments, the method further includes determining, by the at least one processor, the low risk of the cancer risk level where the cancer risk classification score is between 0.0 and 4.9; determining, by the at least one processor, the intermediate risk of the cancer risk level where the cancer risk classification score is between 5.0 and 10.0; and determining, by the at least one processor, the high risk of the cancer risk level where the cancer risk classification score is between 10.1 and 20.0.

In some embodiments, the first stage cancer risk classifier includes: a pre-menopausal first stage cancer risk prediction model having learned pre-menopausal risk classification parameters of the learned risk classification parameters; and a post-menopausal first stage cancer risk prediction model having learned post-menopausal risk classification parameters of the learned risk classification parameters.

In some embodiments, the method further includes determining, by the at least one processor, the low risk of the cancer risk level where the cancer risk classification score is between 0.0 and 4.9; determining, by the at least one processor, the intermediate risk of the cancer risk level where the cancer risk classification score is between 5.0 and 10.0 for a post-menopausal subject; and determining, by the at least one processor, the low risk of the cancer risk level where the cancer risk classification score is between 10.1 and 20.0 for a post-menopausal subject.

In some embodiments, the method further includes determining, by the at least one processor, the low risk of the cancer risk level where the cancer risk classification score is between 0.0 and 4.9; determining, by the at least one processor, the intermediate risk of the cancer risk level where the cancer risk classification score is between 5.0 and 14.0 for a pre-menopausal subject; and determining, by the at least one processor, the high risk of the cancer risk level where the cancer risk classification score is between 14.1 and 20.0 for a pre-menopausal subject. In some embodiments, the subject is diagnosed with a symptomatic or asymptomatic adnexal mass.

### Kits for Detection of Biomarkers for Ovarian Cancer

In another aspect, the invention provides kits for aiding in the diagnosis of ovarian cancer (e.g., identifying ovarian cancer status, detecting ovarian cancer, identifying early stage ovarian cancer, selecting a treatment method for a subject at risk of having ovarian cancer, and the like), which kits are used to detect biomarkers according to the invention. In one embodiment, the kit comprises agents that specifically recognize the biomarkers or combinations of the biomarkers identified in Table 1. In some embodiments, the kit comprises agents that specifically recognize the biomarkers or combinations of the biomarkers identified in Table 1 and markers associated with germline or other DNA mutations identified in connection with ovarian cancer. The kit may contain 1, 2, 3, 4, 5, or more different agents that each specifically recognize one of the biomarkers. In related embodiments, the agents are antibodies, aptamers, Affibodies, hybridization probes and/or fragments thereof.

In another embodiment, the kit comprises a solid support, such as a chip, a microtiter plate or a bead or resin having capture reagents attached thereon, wherein the capture reagents bind the biomarkers of the invention. Thus, for example, the kits of the present invention can comprise mass spectrometry probes for SELDI, such as ProteinChip® arrays. In the case of biospecific capture reagents, the kit can comprise a solid support with a reactive surface, and a container comprising the biospecific capture reagents.

The kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In a further embodiment, such a kit can comprise instructions for use in any of the methods described herein. In embodiments, the instructions provide suitable operational parameters in the form of a label or separate insert. For example, the instructions may inform a consumer about how to collect the sample, how to wash the probe or the particular biomarkers to be detected.

In yet another embodiment, the kit can comprise one or more containers with controls (e.g., biomarker samples) to be used as standard(s) for calibration.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides and polypeptides of the invention, and, as such, may be considered in making and practicing the invention. Useful techniques for particular embodiments will be discussed in the sections that follow.

The following examples are put forth to provide those of ordinary skill in the art with a complete disclosure and description of how to make and use the assay, screening, and therapeutic methods of the invention, and are not intended to limit the scope of what the inventors regard as their invention.

### EXAMPLES

### Example 1: Adnexal Mass Risk Assessment: A Multivariate Index Assay for Malignancy Risk Stratification

Adnexal masses are a common clinical diagnosis in women from the age of adolescence onward, occurring in five to ten percent of women during their lifetimes. Masses are benign in the vast majority of cases (see e.g., Demir R, Marchand G. Adnexal masses suspected to be benign treated with laparoscopy. J. Soc. Laparoendosc. Surg. 16(1), 71-84 (2012)). However, the potential risk of malignancy must be considered.

Benign masses may cause problems due to size, proximity to organs and pain or discomfort. Surgical removal of the mass may be recommended. However, some benign masses may be largely asymptomatic and can be monitored. The benefits of this are clear: the avoidance of an invasive surgery and the associated costs, both financial and in terms of recovery time (see e.g., Farghaly S., Current diagnosis and management of ovarian cysts. Clin. Exp. Obstet. Gynecol. 41(6), 609-612 (2014).).

The current standard of care for a suspected adnexal mass is imaging, typically performed via transvaginal ultrasonography. Imaging may clearly reveal the mass to be benign or malignant based on its physical features, but there are many cases in which the mass is indeterminate (see e.g., Sadowski E, et al. Indeterminate adnexal cysts at US: prevalence and characteristics of ovarian cancer. Radiology 287(3), 1041-1049 (2018)). In these cases, in an asymptomatic patient, follow-up imaging after a period of time is recommended in order to determine if the mass is persistent, stable or has resolved. A risk associated with this approach is delay in detection and management of a malignant lesion (see e.g., Modesitt S, et al. Risk of malignancy in unilocular ovarian cystic tumors less than 10 centimeters in diameter. Obstet. Gynecol. 102(3), 594-599 (2003)).

Adnexal mass risk assessment (AMRA) is a multivariate index assay of serum biomarkers developed to segregate patients with a suspected adnexal mass into three risks of malignancy categories to assist in the decision on whether immediate surgery is recommended. The objective is to use one cutoff to capture a high percentage of the total cancer cases within a small group of high risk patients resulting a clinically actionable positive predictive value and to use a second cutoff to assign a significant portion of the remaining test population into a low-risk (LR) group with a very high negative predictive value.

The ability to develop and evaluate a serum test is often hindered by the lack of properly enrolled clinical samples with inclusion/exclusion criteria representative of the targeted test population and the practical difficulty of missing definitive clinical classifications for those who do not have surgery. For the development as well as independent validation of AMRA, biomarker data were retrospectively analyzed from prospectively collected cohorts of patients diagnosed with adnexal masses and for whom all had definitive pathological classifications from surgery. To project the performance of AMRA on its intended population of patients with adnexal masses prior to the decision of surgery, except for the obvious differences in cancer prevalence, the cancer cases in the patient cohorts used in the study and AMRA's intended population are similar in terms of histologic subtypes, grades, and stages, and the noncancer benign patients in AMRA's intended population were controls. Test performance metrics were estimated, such as positive and negative predictive values of the AMRA high- and low-risk groups, respectively by adjusting for an assumed malignancy prevalence that is typically observed in AMRA's intended populations (see e.g., Molinaro A., Diagnostic tests: how to estimate the positive predictive value. Neuro Oncol. Practice 2(4), 162-166 (2015)).

There are many reasons a woman with an adnexal mass might wish to avoid immediate surgery if it is safe to do so, including financial cost and the recovery time involved in surgery.

However, there is not presently a test on the market specifically aimed at this ‘watch and wait’ population. The purpose of AMRA is to address this need, so that in the future women with adnexal masses might consider delaying surgery.

### Example 1.1: Methods

### Multi-Analyte Panel

Seven serum protein analytes were included as input for AMRA development: ApoA1, B2M, CA125, FSH, HE4, TRF and TT. Subsets of these analytes have been used for multivariate index assay (MIA) and multivariate index assay second generation (MIA2G). For all datasets, the analytes were measured on Roche cobas 6000 (Roche Diagnostics Corp., IN, USA) per manufacturer product package inserts.

### Sample Sets

Data from samples in four separate collections were used in this study. For the derivation of the AMRA algorithm, the training data were from samples that have been previously used for the derivation and validation for the original MIA and the MIAG2 IVDMIA (Ueland F, et al. Effectiveness of a multivariate index assay in the preoperative assessment of ovarian tumors. Obstet. Gynecol. 117, 1289-1297 (2011); Bristow R, et al. Ovarian malignancy risk stratification of the adnexal mass using a multivariate index assay. Gynecol. Oncol. 128, 252-259 (2013); Coleman R, et al. Validation of a second-generation multivariate index assay for malignancy risk of adnexal masses. Am. J. Obstet. Gynecol. 215(1), 82.e1-82.e11 (2016)) tests (‘OVA1 Study’). These samples were originally collected prospectively from 27 Institutional Review Board-approved sites throughout the USA. Inclusion criteria were: women age >18 years, signed informed consent, agreeable to phlebotomy and documented pelvic mass planned for surgical intervention within 3 months of imaging. A pelvic mass was confirmed by imaging (computed tomography, ultrasonography or MRI) prior to enrollment. Exclusion criteria included a diagnosis of malignancy in the previous 5 years (excepting nonmelanoma skin cancers). With the original use of the sample collection to evaluate the utility of MIA and MIAG2 in referring high-risk (HR) patients to be operated by gynecologic oncologists, the collection excluded patient initially enrolled by a gynecologic oncologist. Menopause was defined as the absence of menses for >12 months, or age >50 years for a small number of subjects for whom the menopausal data were missing. Demographic and clinicopathological data were collected on case report forms.

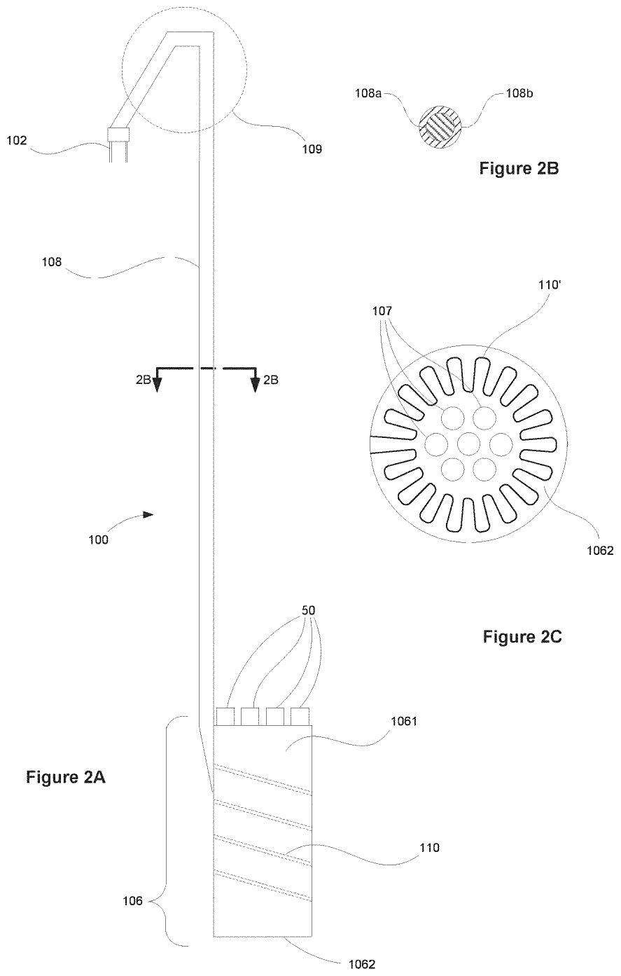

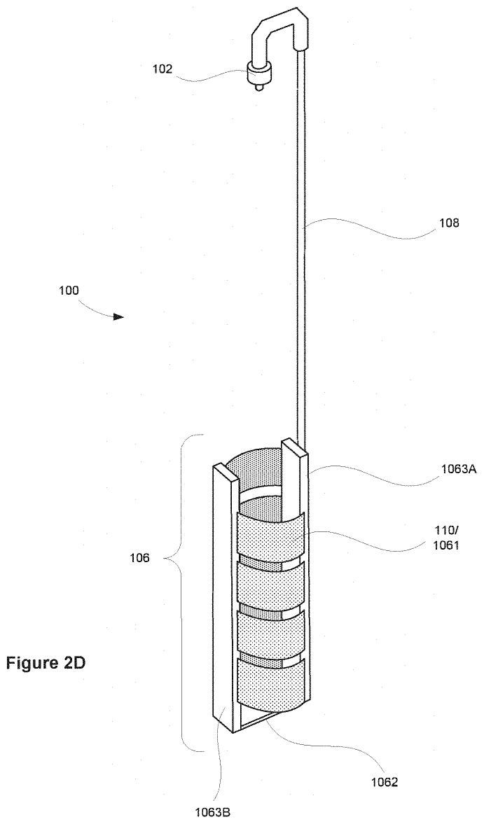

The original prospectively collected sample set represented the actual test population of preoperative risk assessment of adnexal masses. For AMRA algorithm development, a subset (88.36%) of the total samples for whom results of all seven analytes were available. Among the 585 samples, there were 284 premenopausal patients including 54 ovarian cancer cases and 230 benign controls, and 301 postmenopausal patients with 124 cases and 177 controls. FIG. 2 lists relevant demographic and clinicopathological descriptions of the sample sets. The trained AMRA algorithms were then validated on datasets from the remaining three independent Institutional Review Board-approved specimen cohorts: FHCRC #7788, the OVA500 study and OVA1-PS1-CO4.

For FHCRC #7788, patients were prospectively enrolled at gynecologic oncology and benign gynecologic clinics at the Seattle Cancer Care Alliance and the University of Washington Medical Center from 2012 to 2015. Inclusion criteria included women age >18 years, signed informed consent and a documented adnexal mass planned for surgery. An adnexal mass was confirmed by imaging (computed tomography, ultrasonography or MRI) prior to enrollment. Exclusion criteria included pelvic surgery within 6 weeks prior to presentation. Demographic, clinical and pathologic information were collected prospectively. Informed consent was provided by all enrolled patients. For validation of AMRA, a case-control set from the FHCRC #7788 cohort was used based on samples with all seven biomarkers.

The OVA500 study and OVA1-PS1-CO4 had the same enrollment and exclusion criteria as the OVA1 study, except that OVA1-PS1-CO4 included only subjects not yet referred to a gynecologic oncologist even though for whom surgical intervention had been planned. As a result, the prevalence of ovarian cancer in OVA1-PS1-CO4 is lower than that in OVA500.

All samples in each study with known biomarker values for all seven analytes were used in this analysis. Among the cancer cases, 15 samples (0.074% of total samples) with malignancies not involved with the ovary were removed from analysis.

### Model Derivation

AMRA was developed for the intended utility of using two cutoffs to identify a small group of HR patients that captures a large portion of the cancer cases and a relatively large group of LR patients with a high negative predictive value. During model derivation, the desired performance characteristics, in particular, to have an improved sensitivity at very high specificity, was translated and implemented numerically and computationally to influence the derivation and selection of the final models. To assure statistical stability of results, statistical resampling approaches, such as bootstrap sampling of data points within the training sample set and random selection of subset of input analytes, were used.

Two separate predictive models (algorithms) were derived for the pre- and post-menopausal patient populations, respectively. For each algorithm, the training dataset was also used to determine a ‘rule-in’ cutoff to identify HR group and a ‘rule-out’ cutoff to identify relatively LR group. The samples between the two cutoffs were classified as intermediate risk (IR). The selection of cutoffs was driven by the desired performance characteristics based on consensus from clinicians. For example, tradeoff between sensitivity and having a smaller proportion of patients in HR group (and the corresponding positive predictive value (PPV) determined the rule-in cutoff, and similarly, balance between a required high negative predictive value (NPV) and having a sufficiently large LR group were used to select the rule-out cutoff.

### Performance Evaluation

The derived AMRA algorithms and the fixed cutoffs were evaluated on the training set and the three independent validation sets individually and combined. Area under curves from receiver-operating characteristic curve analysis were used to assess the overall performance and to compare with CA125. Sensitivity for the rule-in cutoff (proportion of total cases captured by the HR group) and specificity for the rule-out cutoff (proportion of total benigns in the LR group) were estimated. In addition, positive likelihood ratios (LR+) and negative likelihood ratios (LR−), which are not dependent on prevalence, were also estimated for the HR and LR groups, respectively, and used to provide approximated assessment on changes in post-test probability of cancer from pretest probability.

The samples used for the current study were all from what were originally prospectively collected cohorts. To project the performance of the AMRA algorithms in its intended population, a pretest prevalence of 5 and 10% was used in pre- and post-menopausal test populations, respectively. The projected distribution of benign, low-malignant potential tumors (LMPs), stage I/II cases and stage III/IV cases among the three AMRA risk classification groups were estimated after adjustment for the assumed prevalence. Based on such adjustments, percentage of total test population, post-test cancer prevalence was estimated for the AMRA risk groups, including PPVs and NPVs for the HR and LR groups, respectively.

### Example 1.2: Results

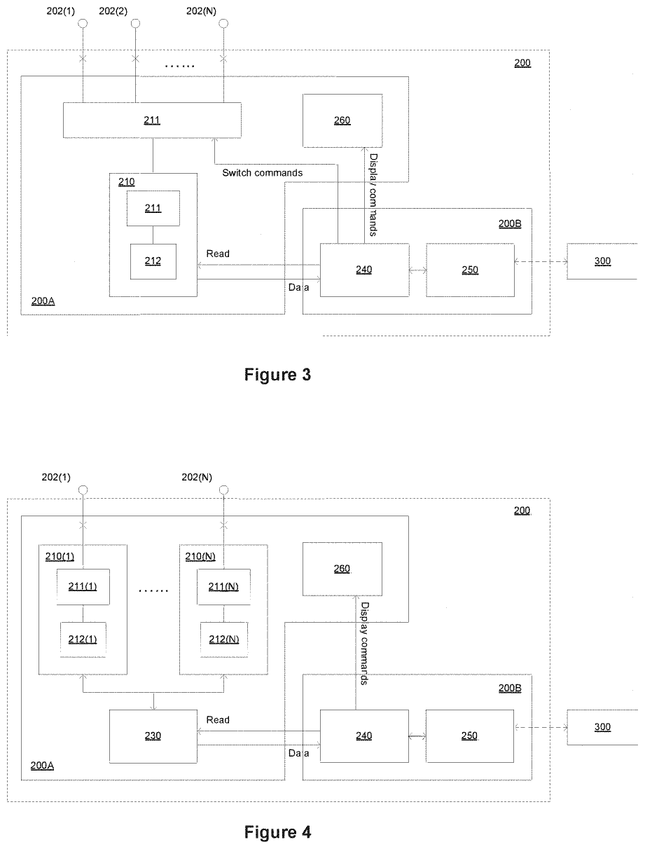

FIG. 3 shows the receiver operating characteristic (ROC) curves of AMRA with comparison to CA125 on pre-menopausal (FIG. 3A) and post-menopausal (FIG. 3B) patients in the training set, and the three validation sets. The performance characteristics of AMRA compared to CA125 is shown by the ROC curve representing sensitivity at very high specificity (leftmost of the plot), which determines the performance characteristics of the rule-in (HR) group (FIG. 3). In particular, FIG. 3 shows the results for AMRA in premenopausal patients for ovarian cancer (p-values=0.01, 0.06, 0.05 and <0.01) as well as for stage I/II invasive ovarian cancer (p-values=0.01, 0.07, <0.01 and <0.01; FIG. 3C) for the training set (OVA1), and the three validation sets (OVA500, FHCRC #7788 and OVA1-PS1-CO4), respectively.

The AMRA rule-in and rule-out cutoff values, at 14.0 IU and 5.0 IU, respectively, for the premenopausal model, and 10.0 IU and 5.0 IU for the postmenopausal model, were established using the training sample set and then fixed in validation. Using the rule-in and rule-out cutoffs, the samples in the three validation sets were combined to estimate and plot the prevalence-adjusted cancer/benign distributions among the three AMRA risk groups (FIG. 4). The raw and prevalence-adjusted proportion of cancer and benign patients within each risk groups are listed in FIGS. 5 and 6. In FIG. 8, both prevalence-independent performance metrics such as sensitivity, specificity, and LR+ and LR−, and the prevalence-adjusted estimates such as percentage of test population, PPV and NPV are provided for the individual and meaningful combinations of risk groups. FIG. 7 plots the prevalence-adjusted projected post-test cancer probabilities of the AMRA risk groups superimposed with an interpolation curve by logistic regression.

At 5% prevalence, the high risk group, 7.9% total, captured 75.9% of invasive malignancies at a positive predictive value of 35.8%. High risk/intermediate risk combined had a sensitivity of 89.7 and 95.6% for pre- and post-menopausal cancers, respectively. The low-risk group, 67.8% total, had a negative predictive value of 99.0%. For both pre- and post-menopausal population, the HR group (7.9 and 10.6% of test population, respectively) captured over two-thirds of the total cancer cases with PPVs at 42.3 and 66.1%, respectively. When LMPs were excluded, the sensitivity of HR both increased to 75% or above. The LR group identified a significant portion of the test population (67.8 and 52.7% for pre- and post-menopausal, respectively) with an NPV close to 99%. Post-test cancer prevalence in the remaining IR group patients was lower than the assumed pretest prevalence (4.0 and 6.3% for all cancer, or 2.1 and 4.8% excluding LMPs, for pre- and post-menopausal, respectively). FIG. 8 also lists the sensitivity and PPV of the combined group of HR and IR to be at 85.9 and 13.3%, respectively for premenopausal patients and 93.1 and 19.7%, respectively, for postmenopausal patients. The sensitivities for invasive cancer only were higher.

### Example 1.3: Discussion

AMRA was developed to segregate patients into three groups with highly differentiating post-test cancer probabilities. Under the assumption that the ovarian cancer cases would be similar in terms of histologic subtypes, grades and stages in patients diagnosed with adnexal masses with or without planned surgery, PPVs and NPVs were estimated using the validation datasets individually (FIG. 4) (Molinaro A. Diagnostic tests: how to estimate the positive predictive value. Neuro Oncol. Practice 2(4), 162-166 (2015)). Adjusting for assumed pretest cancer prevalence, AMRA's potential performance was evaluated for its intended use in the decision of whether immediate surgery is recommended for the individual validation sets. To improve statistical stability of the estimated results, the overall validation results were further estimated using the combined validation data, adjusted to the assumed pretest prevalence (FIGS. 5-8).

The projected post-test cancer probabilities among the AMRA risk groups, such as PPV for the HR group, and NPV for the LR group, are dependent on the assumed pretest cancer prevalence. The ability of AMRA to segregate patients into highly differential and clinically meaningful risk groups is however further supported by the estimated positive and negative likelihood ratios (LR+ and LR−) which are prevalence-independent measures of how a positive or negative test result might alter the probability of disease. For example, using a simplified interpretation of LRs suggested by Steven McGee (McGee S. Simplifying likelihood ratios. J. Gen. Intern. Med. 17(8), 646-649 (2002)) the estimated LR+ of >10.0 for the postmenopausal AMRA HR group in the combined validation samples indicates a potential increase of approximately 45 percentage points in cancer probability. Similarly, the estimated LR− of 0.5 for the postmenopausal AMRA LR group, suggests a post-test decrease in cancer probability of approximately 15 percentage points.

As a multivariate index assay, AMRA's performance was evaluated across multiple histological subtypes and for detecting LMPs and stage I/II invasive ovarian cancers. ROC curve analysis compared AMRA to CA125, as well as in stage I/II ovarian cancer for premenopausal patients for whom the decision of surgery often requires careful consideration.

To aid clinicians in the management of indeterminate masses, key features of AMRA design and actual derivation and implementation included a high sensitivity with its rule-out cutoff resulting a very high NPV for a large portion of the test populations indicated by AMRA as LR. The rule-in cutoff identified a group of patients offering a clinically actionable PPV and captured a majority of the total cancer cases. Based on the independent validation results, the AMRA algorithm with its two cutoffs demonstrated efficiency in clinical management of patients diagnosed with a suspicious adnexal mass, including recommendation for surgery for HR patients, serial monitoring with ultrasound exam for LR patients, and assessment by clinical impression for IR patients.

### Example 2: Multivariate Index Assay Improves the Risk Assessment for Ovarian Cancer and has Utility for Guiding the Clinical Management of Women Diagnosed with Adnexal Mass

### Example 2.1: Background

The need for accurate assessment of cancer risk in women with an adnexal mass prior to their surgical treatment is firmly established (Sanchez-Salcedo M A. Pre-operative assessment of adnexal mass. Obstet Gynecol Int J 2019; 10(1):65-69). From 5-35% of prepubescent females, 10% of pre-menopausal and 30% of post-menopausal women who have an ovarian mass will harbor cancer (Givens V, et al. Diagnosis and Management of Adnexal Mass. Am Fam Physician 2009; 80(8):815-820; Radhamani S, Akhila M V. Evaluation of Adnexal Masses-Correlation of Clinical, Sonological and Histopathological Findings in Adnexal Mass. Int J Sci Studies 2017; 4(11):88-92). While a benign mass can be removed by the obstetrician-gynecologist, a cancer surgery should be performed by a surgical specialist (3. Radhamani S, Akhila M V. Evaluation of Adnexal Masses-Correlation of Clinical, Sonological and Histopathological Findings in Adnexal Mass. Int J Sci Studies 2017; 4(11):88-92; Vemooij F, et al. The outcomes of ovarian cancer treatment are better when provided by gynecologic oncologists and in specialized hospitals. Gynecol Oncol 2007; 105(3)801-812). A preoperative surgical risk assessment ensures the best prognosis for the patient (Glanc P, et al. First international consensus report on adnexal masses-Management Recommendations. J Ultrasound Med 2017; 36:849-863).

For some women diagnosed with a symptomatic adnexal mass, immediate surgery may not be desired or warranted (Suh-Bergmann E, et al. Outcomes from ultrasound follow-up of small complex masses in women over 50. Am J Obstet Gynecol 2014; 211: 623.e1-7; Froyman W, et al. Risk of Complications in patients with conservatively managed ovarian tumors (IOTA5): a 2-year interim analysis of a multicenter, prospective, cohort study. Lancet Oncol 2019; 20(3):448-458; May T, Oza A. Conservative management of adnexal mass. Lancet Oncol 2019; 20(3): p 326-327). In cases of an incidental asymptomatic mass that is found by routine pelvic exam or imaging, surgery may not be the first choice for management. A cancer risk assessment test that could identify low cancer risk subjects for a “wait and watch” management approach would reduce the number of women subjected to clinical workup and surgery.

The diagnostic evaluation of a patient with an adnexal mass includes imaging, usually an ultrasound examination, along with a pelvic exam, and a CA125 blood test. But, the diagnostic accuracy of these modalities, either used alone or as a panel, are not adequate for the detection of early stage ovarian cancers (Lennox G, et al. Effectiveness of the risk of malignancy index and the risk of ovarian malignancy algorithm in a cohort of women with ovarian cancer. Int J Gynecol Cancer 25; 2015; 25: 809-814). During the past 10 years, multivariate index (MIA) assays have been introduced for use in identifying women who are at high risk for ovarian cancer. MIAs consist of a panel of biomarkers with the test results of each biomarker combined into a single score. MIAs, such as the OVA1 (CA125, β2M, Transferrin, Transthyretin and ApoA1) and OVERA (FSH, CA125, HE4, Transferrin, Apo A1) blood tests have been shown to be highly effective in detecting ovarian cancers of all histologic cell types, and at early stage of disease (Ueland F, et al. Obstet Gynecol 2011; 117(6): 1289-1297; Goodrich S, et al. The effect of ovarian imaging on the clinical interpretation of a multivariate index assay. Am J Obstet Gynecol 2014; 211: 65e1-65e11). The Risk of Ovarian Malignancy Algorithm (ROMA), a panel consisting of the CA125 and HE4 tests, is limited to the detection of epithelial ovarian cancers, and is reported to miss a high percentage of early stage cancers (Lennox G, et al. Effectiveness of the risk of malignancy index and the risk of ovarian malignancy algorithm in a cohort of women with ovarian cancer. Int J Gynecol Cancer 25; 2015; 25: 809-814). Additionally, CA125 and ROMA have been shown to perform poorly in identification of malignancy in non-Caucasian women (Dunton C, et al. Ethnic disparity in clinical performance between multivariate index assay and CA125 in detection of ovarian malignancy. Future Oncol. 2019); Dunton C, et al. Multivariate index assay is superior to CA125 and HE4 testing in detection of ovarian malignancy in African American women. Biomarkers In Cancer 2019; 11: 1-4)

As described in Example 1, the AMRA algorithm uses the input of seven biomarkers to provide a cancer risk assessment (Zhang Z, Bullock R, Fritsche H. Adnexal mass risk assessment: a multivariate index assay for malignancy risk stratification. Future Oncol 2019). The AMRA score, is a mathematical combination of the individual biomarker concentrations into a single score. The range of AMRA scores is from 0 to 20.0. The AMRA score was able to stratify symptomatic women with adnexal mass into three cancer risk categories. A high cancer risk group was defined by a high positive predictive value, and a low cancer risk group defined by a high negative predictive value. The remaining subjects were classified as an intermediate risk group. High risk women are typically considered for immediate referral for surgery, and low cancer risk women are considered for a ‘wait and watch’ strategy. However, too many women were classified into the intermediate risk category, raising the question of how to best manage this subgroup of women.

The AMRA test was modified to include additional algorithms, OVA1 (CA125, β2M, Transferrin, Transthyretin and ApoA1) and/or OVERA (FSH, CA125, HE4, Transferrin, Apo A1), which provide an enhanced analysis of subjects included in the intermediate risk group. With the AMRA2 multi-step algorithm, low risk and high risk patients are first identified by the AMRA score of less than 5.0 for low risk, and greater than 10.0 or 14.0, for high risk post-menopausal and pre-menopausal women, respectively. The intermediate risk samples are then subjected to additional algorithms which redefine the intermediate scores as either low risk or high risk. Thus, AMRA2 is able to improve risk assessment of AMRA by eliminating the intermediate risk group by categorizing all subjects as either low risk or high risk, while maintaining both high sensitivity and high specificity for the detection of ovarian cancer.

### Example 2.2: Methods

Biomarker Assays: The seven biomarkers used in the AMRA2 MIA to define cancer risk include: Cancer antigen 125 (CA125), human epididymis protein (HE4), beta-2 microglobulin (B2M), apolipoprotein A-1 (ApoA1), transferrin, transthyretin, and follicle stimulating hormone (FSH). Biomarker assays were performed using the Roche cobas 6000 analyzer, according to the manufacturer's instructions for use.

Algorithm: The AMRA2 test uses the AMRA algorithm to define cutoff scores for low risk and a high risk groups based on set criteria for positive and negative predictive values (see Example 1). The samples falling into the intermediate risk group were reassessed with additional algorithms, OVA1 (CA125, β2M, Transferrin, Transthyretin and ApoA1) OVERA (FSH, CA125, HE4, Transferrin, and Apo A1), derived from unique subsets of the original seven biomarkers, to re-score the intermediate sample as either low or high risk.

The samples were tested with the AMRA algorithm, which includes markers CA125, β2M, Transferrin, Transthyretin and ApoA1, FSH, and HE4, to define scores for low risk and high risk groups. The intermediate group samples were then tested with OVA1, which includes markers CA125, β2M, Transferrin, Transthyretin and ApoA1, to re-score the as either low or high risk. In some samples, after the intermediate group was tested with AMRA and OVA1, the samples were further tested with OVERA, which includes markers FSH, CA125, HE4, Transferrin, and Apo A1, to re-score as either low or high risk. In some tests, after the samples were tested with AMRA, the intermediate group samples were tested with OVERA. In some tests, after the samples were tested with both AMRA and OVERA, the intermediate group samples were further tested with OVA1.

Training and Testing sample sets: The serum samples that were classified as intermediate risk by the AMRA algorithm were used for training and testing of the AMRA2 test. The biomarker data for the sera classified as intermediate risk, was re-analyzed by additional algorithms to reassign the intermediate risk sample as either low risk or high risk, using a cutoff value of 5.0.

Validation sample set: The sample sets used for validation of the AMRA2 MIA were collected under an IRB approved protocol (see Example 1). An independent sample set was collected from 128 women with benign disease, under the same IRB approved requirements, and were used to confirm the high specificity of AMRA2. The serum samples had been stored at −70 degrees C. for a period of up to 2 years. In-house testing confirmed the stability of the seven biomarkers during the storage period.

Procedure: Biomarker data from previously assayed samples was used to train and test the AMRA2 algorithm (Zhang Z, et al. Adnexal mass risk assessment: a multivariate index assay for malignancy risk stratification. Future Oncol 2019). Serum samples used for validation of the AMRA2 MIA were analyzed (ASPiRA Labs), and the test results were used for calculation of the AMRA2 score. AMRA2 scores of less than 5.0, were classified as low risk for both pre- and post-menopausal women. Scores higher than 5.0 were defined as high risk.

### Example 2.3: Results

FIG. 9 shows the categorization of the sample set used for validation of the AMRA2 risk assessment. In total, out of the 596 samples from women with adnexal mass, 23 were characterized with having ovarian cancer. In the first step, the AMRA2 algorithm used cutoff scores to identify low risk (<5), intermediate risk (between 5 and 10 (pre-menopausal), between 5 and 14 (post-menopausal)), and high risk (>10 (pre-menopausal), >14 (post-menopausal)) patients. During this step, AMRA2 identified 45 high risk cases, which identified 18 of the 23 patients identified with ovarian cancer. 391 cases were identified as low risk with 388 cases being benign and 3 cases with ovarian cancer. 160 cases were identified as intermediate risk.

In the second step, the 160 cases identified as being intermediate risk were divided by pre- and post-menopausal status. The intermediate group was then subjected to the OVA1 test, to separate patients into low risk (post-meno<4.4; pre-meno<5), intermediate/borderline risk (post-meno between 4.4-6; pre-meno between 5-7), and high risk (post-meno>6; pre-meno>7) groups. Those women identified in the second step as having intermediate/borderline risk was tested further in a third step with the OVERA test to separate patients into low risk (<5) and high risk (>5) groups, which is not influenced by menopausal status. As a result, two additional cases of ovarian cancer was identified.

Overall, out of 596 women with adnexal mass, 83 were identified as high risk patients with 20 patients out of the total 23 patients identified with ovarian cancer. 63 cases were identified as being false positives. 513 women were identified as low risk, with 510 women with benign masses.

Table 2 further shows the AMRA2 risk assignment for all cancer and benign case in the training set. All subjects are classified as either low or high risk. The original AMRA algorithm categorized 31% of the pre-menopausal and 51% of post-menopausal women into an intermediate risk group.

TABLE 2
Clinical performance of AMRA2 in the AMRA training set.

| Menopausal | | Sensitivity | Specificity | PPV | NPV |
|---|---|---|---|---|---|
| Status | N | (95% CI) | (95% CI) | (95% CI) | (95% CI) |
| All | 545 | 92.03 | 70.52 | 51.42 | 96.31 |
| | | (87.51-96.55) | (66.09-74.95) | (45.18-57.65) | (94.17-98.45) |
| Pre | 270 | 85.00 | 80.87 | 43.59 | 96.88 |
| | | (73.93-96.07) | (75.79-85.95) | (32.59-54.59) | (94.41-99.34) |
| Post | 275 | 94.90 | 57.06 | 55.03 | 95.28 |
| | | (90.54-99.25) | (49.77-64.35) | (47.53-62.53) | (91.25-99.32) |

Table 3 shows the performance parameters for the AMRA2 algorithm in the AMRA test set. The specificity of the AMRA2 in the post-menopausal test set was much better than in the training set (80.1% vs 57.0%). In order to clarify the discrepancy, anew sample set composed of 128 women with adnexal mass who did not have cancer were tested. Of these 128 noncancer women, only 18 were misclassified by AMRA2 as high risk, thus the sensitivity of AMRA2 in this population was 86%.

TABLE 3
Clinical performance of AMRA2 in the AMRA test set.

| Menopausal | | Sensitivity | Specificity | PPV | NPV |
|---|---|---|---|---|---|
| Status | N | (95% CI) | (95% CI) | (95% CI) | (95% CI) |
| All | 1475 | 86.60 | 85.25 | 47.06 | 97.67 |
| | | (81.80-91.39) | (83.30-87.19) | (41.88-52.24) | (96.79-98.56) |
| Pre | 956 | 84.48 | 87.59 | 31.01 | 98.84 |
| | | (75.16-93.80) | (85.40-89.77) | (23.80-38.22) | (98.09-99.59) |
| Post | 539 | 87.50 | 80.15 | 59.80 | 95.00 |
| | | (81.94-93.06) | (76.25-84.04) | (52.99-66.61) | (92.68-97.32) |

Table 4 shows the performance of AMRA2 in the validation set. While the high sensitivity and specificity were confirmed in the validation cohort, the PPV is about half that given by the test set. This reduction in PPV is due to the reduced cancer prevalence of the validation sets. The cancer prevalence in the pre-menopausal group (N=296) was 2.0%, and for the post-menopausal group (N=300) was 5.6% compared to 5.0% and 10.0% prevalence for the pre- and post-menopausal groups in the test sets, respectively. Table 5 gives the demographics for the 596 study subjects in the dataset that was used for AMRA2 validation.

TABLE 4
Clinical performance of AMRA2 in the validation set.

| Menopausal | | Sensitivity | Specificity | PPV | NPV |
|---|---|---|---|---|---|
| Status | N | (95% CI) | (95% CI) | (95% CI) | (95% CI) |
| All | 596 | 86.96 | 89.01 | 24.10 | 99.42 |
| | | (73.19-100) | (86.44-91.57) | (14.90-33.30) | (98.76-100) |
| Pre | 296 | 83.33 | 90.69 | 15.62 | 99.62 |
| | | (53.51-100) | (87.35-94.03) | (3.04-28.21) | (98.80-100) |
| Post | 300 | 88.23 | 87.28 | 29.41 | 99.20 |
| | | (72.92-100) | (83.40-91.16) | (16.91-41.92) | (98.09-100) |

TABLE 5
Validation set demographics and clinicopathologic information.

| Menopausal status | All (N = 596) | | Pre (N = 296) | | Post (N = 300) | |
|---|---|---|---|---|---|---|
| Age, Years | | | | | | |
| Mean (SD) | 48.0 | | 38.6 | | 57.36 | |
| Median | 47 | | 39 | | 57 | |
| Range (min, max) | 18, 87 | | 18, 58 | | 32, 87 | |
| | N | % | N | % | N | % |
| Race/Ethnicity | | | | | | |
| White/Caucasian | 452 | 75.84% | 216 | 72.97% | 236 | 78.67% |
| Black/African- | 69 | 11.58% | 36 | 12.16% | 33 | 11.00% |
| American | | | | | | |
| Hispanic/Latino | 55 | 9.23% | 31 | 10.47% | 24 | 8.00% |
| Asian | 10 | 1.68% | 6 | 2.03% | 4 | 1.33% |
| American Indian or | 1 | 0.17% | 1 | 0.34% | 0 | 0.00% |
| Alaska Native | | | | | | |
| Native Hawaiian or | 3 | 0.50% | 1 | 0.34% | 2 | 0.67% |
| Pacific Islander | | | | | | |
| Other | 6 | 1.01% | 5 | 1.69% | 1 | 0.33% |
| Pathology Diagnosis | | | | | | |
| Benign ovarian | 573 | 96.14% | 290 | 97.97% | 283 | 94.33% |
| conditions | | | | | | |
| Epithelial ovarian | 19 | 3.19% | 4 | 1.35% | 15 | 5.00% |
| cancer | | | | | | |
| Non-epithelial | 2 | 0.34% | 1 | 0.34% | 1 | 0.33% |
| primary ovarian | | | | | | |
| cancer | | | | | | |
| Non-primary | 2 | 0.34% | 1 | 0.34% | 1 | 0.33% |
| ovarian | | | | | | |
| malignancies | | | | | | |
| with involvement | | | | | | |
| of the ovaries | | | | | | |
| Stage (EOC | | | | | | |
| and other primary) | | | | | | |
| Stage I | 9 | 42.86% | 3 | 60.00% | 6 | 37.50% |
| Stage II | 3 | 14.29% | 1 | 20.00% | 2 | 12.50% |
| Stage III | 5 | 23.81% | 0 | 0.00% | 5 | 31.25% |
| Stage IV | 0 | 0.00% | 0 | 0.00% | 0 | 0.00% |
| Not Given | 4 | 19.05% | 1 | 20.00% | 3 | 18.75% |
| Grade (EOC | | | | | | |
| and other primary) | | | | | | |
| 1 | 5 | 23.81% | 1 | 20.00% | 4 | 25.00% |
| 2 | 2 | 9.52% | 1 | 20.00% | 1 | 6.25% |
| 3 | 11 | 52.38% | 2 | 40.00% | 9 | 56.25% |
| Not Given/Other | 3 | 14.29% | 1 | 20.00% | 2 | 12.50% |
| Primary Histology | | | | | | |
| (EOC and other | | | | | | |
| primary) | | | | | | |
| Epithelial (Serous) | 14 | 66.67% | 3 | 60.00% | 11 | 68.75% |
| Epithelial | 2 | 9.52% | 0 | 0.00% | 2 | 12.50% |
| (Mucinous) | | | | | | |
| Epithelial | 1 | 4.76% | 1 | 20.00% | 0 | 0.00% |
| (Clear Cell) | | | | | | |
| Epithelial | 1 | 4.76% | 0 | 0.00% | 1 | 6.25% |
| (Carcinosarcoma) | | | | | | |
| Epithelial (Mixed) | 1 | 4.76% | 0 | 0.00% | 1 | 6.25% |
| Nonepithelial (Sex | 1 | 4.76% | 0 | 0.00% | 1 | 6.25% |
| Cord Stromal) | | | | | | |
| Nonepithelial | 1 | 4.76% | 1 | 20.00% | 0 | 0.00% |
| (Other) | | | | | | |

Table 6 summarizes the classification of the study subjects into the low and high risk groups. For the 6 cancers in the premenopausal group, 5 of the 6 were classified by AMRA2 as High Risk; one of the cancers was classified as low risk. For the 17 cancers in the postmenopausal group, 15 of 17 were classified as high risk and 2 as low risk. Thus, the sensitivity of AMRA2 was 83% and 88% for pre- and postmenopausal women, respectively. For all subjects combined, the sensitivity was 18 of 23 (87%). For comparison purposes, the sensitivity of CA125 in this patient group was 67% and 71%, for pre- and postmenopausal women, respectively. The cutoff values used for assessing the performance of CA125 was 35.0 U/ml for postmenopausal women and 62.0 U/ml for premenopausal women.

TABLE 6
Distribution of AMRA2 risk scores in the validation set.

| | | Malignant | Benign | Total |
|---|---|---|---|---|
| All | High Risk AMRA2 | 20 | 63 | 83 |
| | Low Risk AMRA2 | 3 | 510 | 513 |
| | Total | 23 | 573 | 596 |
| Premenopausal | High Risk AMRA2 | 5 | 27 | 32 |
| | Low Risk AMRA2 | 1 | 263 | 264 |
| | Total | 6 | 290 | 296 |
| Postmenopausal | High Risk AMRA2 | 15 | 36 | 51 |
| | Low Risk AMRA2 | 2 | 246 | 248 |
| | Total | 17 | 282 | 299 |

The AMRA2 test specificity for the premenopausal low risk group was 90%. The test specificity for the postmenopausal low risk group was 87%. For comparative purposes, the specificity of CA125 for pre- and post-menopausal women was 90 and 89%, respectively. The positive predictive value for AMRA2 HR group was 15.62% (5/27) and 29.41% (15/36) for pre- and post-menopausal women, respectively.

### Example 2.4: Discussion

As discussed in Example 1, a group of 956 pre-menopausal women with adnexal mass, in which the prevalence of cancer was 5%, the AMRA MIA demonstrated a three-tier risk stratification of women: A low risk group (67.8% of the population, with ovarian cancer prevalence of 0.6%); a high risk group (7.9% of the population, with a cancer prevalence of 35.8%); and an intermediate risk group (24.3% of the population, with cancer prevalence of 2.1%). In a group of 562 post-menopausal women with a cancer prevalence of 10%, the low risk group contained 52.7% of the women with a cancer prevalence of 0.7%. For 10.6% of women in the high-risk group, the cancer prevalence was 86.6%. The intermediate group had 36.7% of the women and a cancer prevalence of 4.8% (see Example 1; see also Zhang et al. Adnexal mass risk assessment: a multivariate index assay for malignancy risk stratification. Future Oncol 2019, which is incorporated by reference herein in its entirety).

In these same two groups of pre- and post-menopausal women, the AMRA2 two-step algorithm eliminated the intermediate risk group. In so doing, the AMRA2 sensitivity and specificity was improved to 86.60% and 85.25% respectively.

In this current study of 596 women with adnexal mass, in which the cancer prevalence was 3.8%, AMRA2 resulted in a sensitivity of 86.96% and specificity of 89.01%. The sensitivity of AMRA2 was significantly better than CA125, while the specificity of the two tests were similar.

### Example 3: Multivariate Index Assay for Ovarian Cancer Risk Assessment in High Risk Women

Various biomarkers have been proposed for the early detection of ovarian cancer. Women with an adnexal mass have a 10% lifetime risk of developing a malignant tumor, while the risk of ovarian cancer in women who have germline gene mutations have a much higher risk. OVA1 and OVERA multivariate index assays (MIA) (Aspira Lab) were previously developed for assessing cancer risk in women who present with an adnexal mass. Since these masses are not subject to biopsy, ultrasound examination of the mass and the OVA1 biomarker test provide risk assessment for malignancy and guide the clinical management of the patient. Unfortunately, there is no imaging or biomarker test that can effectively detect ovarian cancer in the asymptomatic high risk patient. Thus, there is need for an improved diagnostic test.

As discussed in Example 2, the AMRA2 MIA uses seven biomarkers (ApoA1, CA125, β2M, transferrin, transthyretin, FSH, and HE4) and multiple algorithms to generate a risk score which ranges from 0 to 20. Women with AMRA2 risk scores of less than 5.0 are defined as low risk, which qualify them for a “watch and wait” strategy using serial ultrasound exams and biomarker testing. A high risk AMRA2 score (5.0 or greater) defines women who need consideration for immediate surgery. The probability of cancer increases as the score increases, and can guide the physician in making appropriate surgical decisions.

The AMRA2 blood test was developed to meet the need of early cancer detection in high risk women, defined as women who have an asymptomatic adnexal mass, those who have germ line mutation (e.g., BRCA1/2) or those with other DNA mutations associated with ovarian cancer. The AMRA2 blood test was used to identify which high risk women with symptomatic adnexal mass need immediate surgery from those women who can delay surgery, or perhaps avoid surgery. Three key areas were evaluated: 1) the use of AMRA2 for early detection of cancer in women with symptomatic adnexal mass; 2) to initiate a new clinical use, that is the early detection of ovarian cancer in women with symptomatic adnexal mass; 3) to establish AMRA2 as a monitoring test to guide the prophylactic surgery of women with germline and/or somatic gene mutations and/or aberrant methylation (e.g., hypermethylation or hypomethylation) of genes that are associated with breast and/or ovarian cancer. Several genes associated with breast and/or ovarian cancer (BOC) include, but are not limited to: Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS) BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P161NK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1).

A prospective study was performed to validate the sensitivity, specificity, positive predictive value and negative predictive values of the AMRA2 blood test for cancer risk stratification of women diagnosed with symptomatic adnexal mass, but in whom the ultrasound exam is not definitive for malignancy. Women with pelvic symptoms frequently are shown by ultrasound exam to have an adnexal mass. In about 10% of cases, the mass is found to be malignant and immediate surgery, performed by the gynecologic specialist, is recommended. However, in many of these cases, the ultrasound exam is not definitive, and if the mass is benign, surgery can be delayed. In some cases, the benign mass resolves with no treatment and surgery can be avoided. AMRA2 was designed to segregate patients into high and low risk groups, with the aim of identifying which women can be directed to expectant management in a wait and watch group, while the high risk patients can be directed to immediate surgery.

A monitoring study was performed to define the role of serial testing with AMRA2 for the early detection of cancer in women diagnosed with an asymptomatic adnexal mass. On occasion, an adnexal mass is diagnosed in a woman who has no pelvic symptoms. In this study, women were given serial ultrasound exams and biomarker testing with CA125 and AMRA2. Since ovarian cancer can occur in women with adnexal mass, irrespective of the presence of symptoms, these women were currently managed in a watchful waiting scenario, and monitored in a serial fashion with ultrasound exams and serum CA125.

A second monitoring study was performed to define the role of serial testing with AMRA2 for the early detection of cancer in women who have a germline mutation (e.g., BRCA1/2) or a somatic mutation associated with the development of breast and/or ovarian cancer and/or aberrant methylation (e.g., hypermethylation or hypomethylation) of a breast and/or ovarian cancer gene. Women with key germline or somatic gene mutations or aberrant methylation (e.g., hypermethylation or hypomethylation) have a very high risk of breast and ovarian cancer. For ovarian cancer, CA125 is currently the only blood test used to monitor women for the development of ovarian cancer. Genes associated with breast and/or ovarian cancer were screened using cell-free circulating tumor DNA (cftDNA) as a non-invasive diagnostic tool. CfTDNA was isolated from serum obtained from the subject and checked for single-nucleotide variations (SNVs) or copy number alterations using targeted next-generation sequencing (NGS), with further validation of results by checking respective formalin-fixed paraffin-embedded tumor tissues using NGS or PCR for the same genetic alterations.

For the above studies, approximately 100 women with symptomatic adnexal mass and 100 women with asymptomatic adnexal mass were evaluated. Women with BRCA mutations were monitored until 50 women had a surgical outcome assessment.

Serum samples used in the AMRA2 validation study and the two monitoring studies were collected prospectively from women who were 18 years or older and diagnosed with an ovarian adnexal mass, or in follow-up due to the presence of BRCA1/2 and other DNA mutations and/or aberrant methylation (e.g., hypermethylation or hypomethylation). Retrospectively collected serum samples, obtained in IRB approved patient monitoring studies and maintained in a serum repository was used in this study.

The patient samples were coded and stored at −70° C. until testing. At the time of testing, the samples were defrosted and placed in a coded aliquot in the analyzer. The serum samples were assayed to determine the AMRA2 score. The AMRA2 score was reported to the physician to guide the patient to immediate surgery or to expectant management. Women directed to the watch and wait group were tested in a serial fashion with ultrasound, CA125 and AMRA2 for a period of one year. All women who had a surgical outcome (e.g., positive or negative for cancer detection) were used to define the true positive rate, false positive rate, true negative rate and false negative rate. The performance data from this study was used to validate the clinical utility of AMRA2 for guiding the clinical management of women with adnexal mass.

Women who had asymptomatic pelvic mass and women with key germline and/or somatic gene mutations and/or aberrant methylation (e.g., hypermethylation or hypomethylation) were tested in a serial fashion with ultrasound, AMRA2 and CA125. At the end of the monitoring period, defined by the doctor and patient, surgery was performed to remove the adnexal mass, or for the women with germline and/or somatic mutations and/or aberrant methylation (e.g., hypermethylation or hypomethylation), prophylactic surgery was performed to remove ovaries and fallopian tubes.

### Example 4: Multifactorial Risk Assessment for Breast & Ovarian Cancer Risk Detection

### Example 4.1: Background

An estimated 1.2 to 1.3 million women in the United States with breast or ovarian cancer who qualified for genetic testing failed to receive it; and more than 85% of patients with breast cancer and 80% of patients with ovarian cancer never even discussed genetic testing with their physicians (Christopher P. Childers, et al., National Estimates of Genetic Testing in Women With a History of Breast or Ovarian Cancer, J. Clin Oncol., Aug. 18, 2017). About 10% of women diagnosed with breast and/or ovarian cancer is due to a hereditary cause.

In 2017, an NCI study reported that 25% of women with breast cancer and 33% of women with ovarian cancer underwent genetic testing for known harmful variants in breast and ovarian cancer susceptibility genes. Patients who did receive genetic testing, 8% of breast cancer patients and 15% of ovarian cancer patients had “actionable” gene variants, i.e. variants that might warrant changes in treatment, screening and risk-reduction strategies (Allison W. Kurian, et al., Genetic Testing and Results in a Population-Based Cohort of Breast Cancer Patients and Ovarian Cancer Patients, J. Clin Oncol., Apr. 9, 2019). Of those genes, only twelve account for risk-reducing surgical methods, considered as ‘treatment’ (NCCN guidelines on ovarian cancer 1.2019). This identifies the need to continue efforts to refine the remaining 24+ genes with known harmful variants associated with Breast and Ovarian Cancer (BOC) and create a better treatment plan. Of those twelve genes, BRCA is the most commonly associated gene and only accounts for ˜15-20% of hereditary cancers. Whereas the remaining lower prevalent genes account for ˜18%, leaving a rough estimate of 60% to chance (Thomas Paul Slavin, et al., Clinical application of multigene panels: challenges of next-generation counseling and cancer risk management, Front. Oncol., 29 Sep. 2015). For those women that are diagnosed with a late-stage BOC, genetic testing is usually secondary with a negative finding due to small gene panels, or not offered at all.

Ovarian cancer is hard to diagnose, as it is considered a silent disease. There is no known non-invasive diagnostic test that can measure somatic detection without invasive removal of the tumor and pathology assessment. Current methods used to diagnose are a Trans Vaginal Ultra Sound (TVUS), the most common type of U/S used when symptoms require an image to rule out an ovarian mass, only as a diagnostic test if the symptoms justify it.

Multivariate index assays (e.g., OVA1 and OVERA) have been developed for assessing cancer risk in women who present with an adnexal mass. Since these masses are not subject to biopsy, ultrasound examination of the mass and biomarker tests provide risk assessment for malignancy and guide the clinical management of the patient. Unfortunately, there is no imaging or biomarker test that can effectively detect ovarian cancer in asymptomatic high-risk patients.

One of the challenges is the heterogeneity in the source of the malignancy. Malignant tumors of the ovary may arise from multiple sources (i.e. germ cells, stromal cells, epithelial cells or mesenchymal tissues). Some cases are sporadic and some cases of ovarian cancers can be inherited in families with HBOC or rare genetic mutations, for example Lynch Syndrome. Management and treatment approaches to ovarian cancer are dependent on the pathology of the tumor. There is no single test method in diagnosing ovarian cancer and how best to manage the disease. Currently symptomatic women go through an extensive work-up, seeing a variety of specialists before they receive a surgical assessment from a gynecological oncologist, which allows for the cancer to progress into late-stage with little to no chance of survival. Thus, there is a need for a less-invasive diagnostic test to detect ovarian cancer early in women and that identifies both germline and sporadic risk from developing tumors.

To develop a test for diagnosing patients that are high-risk for ovarian cancer and to develop a gene susceptibility panel and develop an early-screening measurement from cell-tumor DNA (ctDNA) as tested in the blood, tumor profiles were obtained from women either with 1) a symptomatic adnexal mass, 2) an asymptomatic mass found incidentally at pelvic exam, 3) an asymptomatic having genetic testing for HBOC and no sign of an adnexal mass and/or 4) family history or genetic abnormality (germ line and somatic DNA mutation) associated with ovarian cancer.

### Example 4.2: Methods and Procedures

### Sample Collection

Due to the biological nature of the cells, ovarian cancer patient blood samples were collected in 3 separate collection tubes with different mediums to ensure high-integrity collection: (1) standard EDTA (lavender top), (1) PAX (cffDNA) and (1) Tiger top (serum) tube.

The EDTA tube is an anti-coagulant collection apparatus to preserve the morphology of the cellular elements, lymphocytes, that harbor cells containing your DNA. This is a standard tube used for genetic testing.

The PAX Blood ccfDNA tube: is designed specifically to collect whole blood that stabilizes the concentration of circulating cell-free DNA in plasma. This type of tube is important to collect high-integrity ccfDNA.

Tiger top tube contains an anticoagulant, but contains a clot activator and serum separator gel. This allows for the accelerated clotting of the whole blood in order to separate out serum that holds large cellular subunits, such as the proteins tested with OVA1 and/or OVERA assays.

The blood samples collected were then tested using HBOC, OVERA, and/or OVA1 assays.

### Collection of Whole Blood

Whole blood will be collected in 1 EDTA tube and 1 PAX tube of ˜5 mL of blood per tube.

### Collection of Serum

Serum will be collected from 8.5 mL venous draw of the patient's blood by a standard venipuncture procedure using a Serum Separation Tube (Tiger top).

### Collection of a FFPE Tissue

Tissue collected during surgical removal of adnexal mass will be received as a formalin-fixed paraffin embedded (FFPE) slide at a minimum of 20% tumor content. One H&E slide will also be collected to assess tumor content.

### Clinical Samples

All samples will be properly processed to obtain biological material of interest following standard operating procedures (SOPs). Extra material from the serum sample collected for the OVA test offering will be commercial tested and stored at −20° C. until further testing.

The EDTA blood collection tube for germline and/or somatic testing of HBOC will be mixed thoroughly and a 1 mL aliquot will be removed and placed in a tube and stored at 4° C. until further testing. The remaining blood will be processed following laboratory SOPs.

An additional EDTA blood tube will be provided to be processed for plasma and to achieve cell-free DNA collection. These samples will be processed immediately following laboratory SOPs and stored at −20° C. until further processing.

### OTHER EMBODIMENTS

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents, publications, and accession numbers mentioned in this specification, including, but not limited to, Zhang Z, et al. Adnexal mass risk assessment: a multivariate index assay for malignancy risk stratification, Future Oncol 2019, are herein incorporated by reference to the same extent as if each independent patent, publication, and accession number was specifically and individually indicated to be incorporated by reference.

What is claimed is:

1. A panel for pre-operatively assessing a subject's risk of having ovarian cancer, the panel comprising and or consisting of markers Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH), and one or more markers selected from the group consisting of Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), 06-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P16INK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1);
Transthyretin/prealbumin (TT), Apolipoprotein A1 (ApoA1), β2-Microglobulin (β2M), Transferrin (Tfr), Cancer Antigen 125 (CA125), and one or more markers selected from the group consisting of Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), 06-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P16INK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1); or
Apolipoprotein A1 (ApoA1), Transferrin (Tfr), Cancer Antigen 125 (CA125), Human epididymis protein 4 (HE4), follicle stimulating hormone (FSH), and one or more markers selected from the group consisting of Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), 06-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P16INK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1).

2. A panel for pre-operatively assessing a subject's risk of having ovarian cancer, the panel comprising or consisting of markers Transthyretin/prealbumin (TT), Apolipoprotein A1 (ApoA1), β2-Microglobulin (β2M), Transferrin (Tfr), Cancer Antigen 125 (CA125), and Breast Cancer 1 (BRCA1);
Transthyretin/prealbumin (TT), Apolipoprotein A1 (ApoA1), β2-Microglobulin (β2M), Transferrin (Tfr), Cancer Antigen 125 (CA125), and Breast Cancer 2 (BRCA2);
Transthyretin/prealbumin (TT), Apolipoprotein A1 (ApoA1), β2-Microglobulin (β2M), Transferrin (Tfr), Cancer Antigen 125 (CA125), Breast Cancer 1 (BRCA1) and Breast Cancer 2 (BRCA2);
Apolipoprotein A1 (ApoA1), Transferrin (Tfr), Cancer Antigen 125 (CA125), HE4, follicle stimulating hormone (FSH), and Breast Cancer 1 (BRCA1);
Apolipoprotein A1 (ApoA1), Transferrin (Tfr), Cancer Antigen 125 (CA125), HE4, follicle stimulating hormone (FSH), and Breast Cancer 2 (BRCA2);
Apolipoprotein A1 (ApoA1), Transferrin (Tfr), Cancer Antigen 125 (CA125), Human epididymis protein 4 (HE4), follicle stimulating hormone (FSH), Breast Cancer 1 (BRCA1) and Breast Cancer 2 (BRCA2);
Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), follicle stimulating hormone (FSH), and Breast Cancer 1 (BRCA1);
Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), follicle stimulating hormone (FSH), and Breast Cancer 2 (BRCA2); or
Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), follicle stimulating hormone (FSH), Breast Cancer 1 (BRCA1), and Breast Cancer 2 (BRCA2).

3. The panel of claim 1, wherein each of the markers are bound to a separate capture reagent.

4. The panel of claim 3, wherein the capture reagents are attached to a solid support.

5. The panel of claim 4, wherein the solid support is a plate, chip, beads, microfluidic platform, membrane, planar microarray, or suspension array

6. The panel of claim 3, wherein the capture reagent is an antibody, aptamer, Affibody, hybridization probe and/or fragments thereof.

7. The panel of claim 3, wherein each capture reagent specifically binds to one of the markers.

8. The panel of claim 1 for use in a method for pre-operatively assessing a subject's risk of having ovarian cancer.

9. A method for pre-operatively assessing a subject's risk of having ovarian cancer, the method comprising characterizing markers in a biological sample from the subject using the panel of claim 1.

10. A method for pre-operatively assessing a subject as having a high or a low risk of ovarian cancer, the method comprising,
(a) characterizing markers comprising or consisting of
Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a biological sample derived from the subject to determine a first score, wherein the first score identifies the subject as having a high, intermediate or low cancer risk; and
(b) characterizing markers comprising or consisting of CA125, β2M, Tfr, TT and ApoA1 or FSH, CA125, HE4, Tfr, and ApoA1 in the biological sample derived from the subject identified by the first score as having an intermediate cancer risk to determine a second score, wherein the second score identifies the subject as having a low or high cancer risk.

11. A method for pre-operatively assessing a subject as having a high or low risk of ovarian cancer, the method comprising,
(a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a biological sample derived from the subject to determine first score, wherein the first score identifies the subject as having a high, intermediate or low cancer risk;
(b) characterizing markers comprising or consisting of CA125, β2M, Tfr, TT and ApoA1 in the biological sample derived from the subject identified by the first score as having an intermediate cancer risk to determine a second score, which identifies the subject as low, intermediate, or high risk; and
(c) characterizing markers comprising or consisting of FSH, CA125, HE4, Tfr, and ApoA1 in the biological sample derived from the subject identified by the second score as having an intermediate or high cancer risk to determine a third score, wherein the third score identifies the subject as having a low or high cancer risk.

12. A method for pre-operatively assessing an asymptomatic subject, the method comprising,
(a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a biological sample derived from the subject to determine first score, wherein the first score identifies the subject as having a high, intermediate or low cancer risk; and
(b) characterizing one or more markers selected from the group consisting of Breast Cancer 1 (BRCA1), Breast Cancer 2 (BRCA2), Ataxia-Telangiesctasia mutated (ATM), BRCA1 Associated Ring Domain 1 (BARD1), BRCA1 Interacting Protein C-terminal Helicase 1 (BRIP1), Cadherin-1 (CDH1), Checkpoint Kinase 2 (CHEK2), Epithelial cell adhesion molecule (EPCAM), MutL homolog 1 (MLH1), MutS Homolog 2 (MSH2), MutS Homolog 6 (MSH6), Nibrin (NBN), partner and localizer of BRCA2 (PALB2), Phosphatase and tensin homolog (PTEN), RAD51 paralog D (RAD51D), Serine/Threonine Kinase 11 (STK11), Tumor protein p53 (TP53), Kirsten rat sarcoma viral oncogene homolog (KRAS), BRCA1 A complex subunit abraxas 1 (ABRAXAS1 or FAM175A), RAC-alpha serine/threonine-protein kinase (AKT1 or Protein Kinase B), Adenomatous polyposis coli (APC), axis inhibition protein 2 (AXIN2), Bone Morphogenetic Protein Receptor Type 1A (BMPR1A), proto-oncogene B-Raf (BRAF), Cell Division Cycle 25C (CDC25), Cyclin Dependent Kinase Inhibitor 2A (CDKN2A), Cyclin-dependent kinase 4 (CDK4), Catenin beta-1 (CTNNB1), helicase with RNase motif (DICER1), Erb-B2 Receptor Tyrosine Kinase 2 (ERBB2), Excision Repair Cross-Complementation Group 6 (ERCC6), Fanconi anemia complementation group M (FANCM), Fanconi anemia complementation group C (FANCC), Meiotic Recombination 11 (MRE11), mutY DNA glycosylase (MUTYH), Neurofibromin 1 (NF1), Endonuclease III-like protein 1 (NTHL1), Phosphatidylinositol-4,5-Bisphosphate 3-Kinase Catalytic Subunit Alpha (PIK3CA), Postmeiotic segregation Increased 2 (PMS2), Protein phosphatase 2 regulatory subunit A alpha (PP2R1A), Protein Kinase DNA-Activated Catalytic Subunit (PRKDC), DNA Polymerase Delta 1 Catalytic Subunit (POLD1), RAD50 homolog (RAD50), RAD51 Paralog C (RAD51C), Ring Finger Protein 43 (RNF43), Succinate Dehydrogenase Complex Iron Sulfur Subunit B (SDHB), Succinate Dehydrogenase Complex Subunit D (SDHD), SWI/SNF Related Matrix Associated Actin Dependent Regulator Of Chromatin Subfamily A Member 4 (SMARCA4), X-ray repair complementing defective repair in Chinese hamster cells 2 (XRCC2), Werner syndrome ATP-dependent helicase (WRN or RECQL), Cell Division Cycle 73 (CDC73), Polypeptide N-Acetylgalactosaminyltransferase 12 (GALNT12), Gremlin 1 (GREM1), Homeobox B13 (HOXB13), MutS Homolog 3 (MSH3), DNA Polymerase Epsilon Catalytic Subunit (POLE), RAD51 Recombinase (RAD51), RAD50 Interactor 1 (RINT1), 40S ribosomal protein S20 (RSP20), SLX4 Structure-Specific Endonuclease Subunit (SLX4), SMAD Family Member 4 (SMAD4), Dual specificity protein kinase TTK (TTK), Ras association domain family 1 isoform A (RASSFlA), Runt-related transcription factor 3 (RUNX3), Tissue factor pathway inhibitor 2 (TFPI2), Secreted frizzled-related protein 5 (SFRP5), Opioid-binding protein/cell adhesion molecule (OPCML), O⁶-alkylguanine DNA alkyltransferase (MGMT), Cadherin 13 (CDH13), sulfatase 1 (SULF1), Homeobox A9 (HOXA9), Homeobox A11 (HOXAD11), Claudin 4 (CLDN4), T-cell differentiation protein (MAL), Brother of Regulator of Imprinted Sites (BORIS), ATP-binding cassette super-family G member 2 (ABCG2), Tubulin Beta 3 Class III (TUBB3), Methylation controlled DNAJ (MCJ), synucelin-γ (SNGG), alternative reading frame tumor suppressor (P14ARF), cyclin-dependent kinase inhibitor 2A (CDKN2A or P161NK4A), Cyclin-dependent kinase 4 inhibitor B (CDKN2B or P15), Death-associated protein kinase 1 (DAPK), Calcium channel voltage-dependent T type alpha 1G subunit (CACNA1G or MINT31), Retinoblastoma-interacting zinc-finger protein 1 (RIZ1), and target of methylation-induced silencing 1 (TMS1) in the biological sample derived from the subject identified by the first score as having a high, intermediate or low cancer risk, wherein the presence of one or more mutations in one or more markers or the presence of an aberrant methylation in one or more markers identifies the subject as having a higher [increased] cancer risk relative to a subject that does not have a mutation or an aberrant methylation in the one or more markers.

13. A method for pre-operatively monitoring a subject with one or more mutations or an aberrant methylation in one or more germline and/or somatic markers, the method comprising:
(a) characterizing markers comprising or consisting of Transthyretin/prealbumin (TT), Apolipoprotein A1 (ApoA1), β2-Microglobulin (β2M), Transferrin (Tfr), Cancer Antigen 125 (CA125), Human epididymis protein 4 (HE4), and follicle stimulating hormone (FSH) in a first biological sample derived from the subject to determine a first score at a first time point, wherein the first score identifies the subject as having a high, intermediate or low ovarian cancer risk; and
(b) repeating step (a) in one or more biological samples from the subject identified as having an intermediate or low ovarian cancer risk at one or more time points, thereby monitoring the subject.

14. The method of claim 12, wherein the one or more germline markers are BRCA1 and/or BRCA2.

15. The method of claim 8, wherein the one or more markers are characterized by detecting cell-free tumor DNA (cftDNA).

16. The method of claim 8, wherein the capture reagent is an antibody, aptamer, Affibody, hybridization probe and/or fragments thereof.

17. The method of claim 8, wherein the markers are characterized by immunoassay, sequencing and/or nucleic acid microarray.

18. The method of claim 8, wherein the method further characterizes one or more clinical biomarkers of ovarian cancer risk in the subject, wherein the one or more clinical biomarkers are selected from group consisting of age, pre-menopausal status, post-menopausal status, ethnicity, pathology, adnexal mass diagnosis, family history, physical examination, imaging results, and/or history of smoking, wherein the one or more clinical biomarkers further identifies the subject as having a low or high cancer risk.

19. A method for classifying a subject's risk of having ovarian cancer, the method comprising:
receiving, by at least one processor, a first panel signal representing a marker spectrum peak detected for each marker of a panel comprising or consisting of markers Transthyretin/prealbumin (TT), Apolipoprotein A1 (ApoA1), β2-Microglobulin (β2M), Transferrin (Tfr), Cancer Antigen 125 (CA125), HE4, and follicle stimulating hormone (FSH) and one or more markers selected from the group consisting of Breast Cancer 1 (BRCA1), Breast Cancer 2 (BRCA2), ATM, BARD1, BRIP1, CDH1, CHEK2, EPCAM, MLH1, MSH2, MSH6, NBN, PALB2, PTEN, RAD51D, STK11, TP53, KRAS, ABRAXAS1, AKT1, APC, AXIN2, BMPR1A, BRAF, CDC25, CDKN2A, CDK4, CTNNB1, DICER1, ERBB2, ERCC6, FANCM, FANCC, MRE11, MUTYH, NF1, NTHL1, PIK3CA, PMS2, PP2R1A, PRKDC, POLD1, RAD50, RAD51C, RNF43, SDHB, SDHD, SMARCA4, XRCC2, WRN, CDC73, GALNT12, GREM1, HOXB13, MSH3, POLE, RAD51, RINT1, RSP20, SLX4, SMAD4, TTK, RASSFlA, RUNX3, TFPI2, SFRP5, OPCML, MGMT, CDH13, SULF1, HOXA9, HOXAD11, CLDN4, MAL, BORIS, ABCG2, TUBB3, MCJ, SNGG, P14ARF, P16INK4A, DAPK, P15, MINT31, RIZ1, and TMS1;
utilizing, by the at least one processor, a first stage cancer risk classifier to predict a cancer risk classification score representative of a predicted risk of developing ovarian cancer, the cancer risk classification score being based on learned risk classification parameters and the first panel signal;
determining, by the at least one processor, a cancer risk level associated with the cancer risk classification score, the cancer risk level selected from one of at least the selection comprising low risk, intermediate risk and high risk; and
generating, by the at least one processor, a cancer risk level prediction at a computing device associated with a care provider indicative of the cancer risk level of the subject.

20. A system comprising the at least one processor configured to execute instructions causing the at least one processor to perform the method of claim 19.

21. A non-transitory computer readable medium storing thereon software, the software comprising program instructions configured to cause the at least one processor to perform the method of claim 19.

22. A kit comprising:
(a) the panel of markers of claim 1; and
(b) instructions for using the panel for pre-operatively assessing a subject's risk of having ovarian cancer.
